(19) United States
(12) Patent Application Publication
(10) Pub. No.: US 2010/0143302 A1
(43) Pub. Date: Jun. 10, 2010

(54) Recombinant Adenoviruses Based on Serotype 26 and 48, and Use Thereof

(75) Inventors: Menzo Jans Emko Havenga, Alphen aan den Rijn (NL); Dan H. Barouch, Brookline, MA (US)

Correspondence Address: TRASKBRITT, P.C., P.O. BOX 2550, SALT LAKE CITY, UT, 84110, US

(73) Assignees: Crucell Holland B.V.; Beth Israel Deaconess Medical Center, Inc

(21) Appl. No.: 12/225,259

(22) PCT Filed: Mar. 15, 2007
(86) PCT No.: PCT/EP2007/052463

§ 371 Date: Sep. 16, 2008

Related U.S. Application Data
(60) Provisional application No. 60/782,918, filed on Mar. 16, 2006.

(51) Int. Cl. A61K 45/00; C07H 21/00; C12N 7/01; C12N 15/74; A61P 37/04

(52) U.S. Cl. 424/93.2; 536/23.1; 536/23.5; 536/23.72; 536/23.7; 435/235.1; 435/320.1

(57) ABSTRACT

The present application relates to recombinant adenoviruses, more in particular those that encounter low levels of pre-existing neutralizing activity in hosts that are in need of treatment or vaccination. Particularly, the invention relates to recombinant vectors derived from two subgroup D adenoviruses: Ad26 and Ad48.

### FIELD OF THE INVENTION

The invention relates to the field of medicine. More in particular, the invention relates to the field of vaccination using recombinant gene delivery vehicles, such as replication-defective adenoviruses that encounter low levels of pre-existing neutralizing activity in the host.

### BACKGROUND OF THE INVENTION

Recombinant adenoviral vectors are widely applied for gene therapy applications and vaccines. To date, 51 different human adenovirus serotypes have been identified. The subgroup C adenoviruses have been most extensively studied for gene delivery applications such as gene therapy; especially serotype 2 and 5 (Ad2 and Ad5) are generally used in the art. Recombinant Ad5 is used for different applications, including vaccination. Importantly, Ad5 vector-based vaccines have been shown to elicit potent and protective immune responses in a variety of animal models. Large-scale clinical trials for HIV vaccination are ongoing in which Ad5-based recombinant vectors are being tested for efficacy (WO 01/02607; WO 02/22080; Shiver J W et al. 2002 Nature 415:331; Letvin N L et al. 2002 Annu Rev Immunol 20:73; Shiver J W and Emini E A. 2004 Annu Rev Med 55:355). However, the utility of recombinant Ad5 vector-based vaccines for HIV and other pathogens will likely be significantly limited by the high seroprevalence of Ad5-specific neutralizing antibodies (NAbs) in human populations. The existence of anti-Ad5 immunity has been shown to suppress substantially the immunogenicity of Ad5-based vaccines in studies in mice and rhesus monkeys. Early data from phase-1 clinical trials show that this problem may also occur in humans (Shiver J W. 2004 Development of an HIV-1 vaccine based on replication-defective adenovirus. Keystone Symposium on HIV Vaccine Development: Progress and Prospects, Whistler, British Columbia, Canada).

One promising strategy to circumvent the existence of pre-existing immunity in individuals previously infected-, or treated with the most common human adenoviruses (such as Ad5), involves the development of recombinant vectors from adenovirus serotypes that do not encounter such pre-existing immunities. Human recombinant replication-defective adenoviral vectors that were successfully generated were based on serotypes Ad11, Ad35, and Ad49 (WO 00/70071, WO 02/40665, WO 2004/037294, and patent application U.S. Ser. No. 11/140,418; Vogels R et al. 2003. J Virol 77:8263; Holterman L et al. 2004. J Virol 78:13207; which disclosures and applications are incorporated herein by reference, in their entirety). Others have found that also adenovirus 24 (Ad24) may be of particular interest as it is shown to act as a ‘rare’ serotype (WO 2004/083418).

A similar strategy is based on the use of simian adenoviruses since these do not typically infect humans and exhibit a low seroprevalence in human samples. They are however applicable for human use since it was shown that these viruses could infect human cells in vitro (WO 03/000283; WO 2004/037189).

It was found that Ad35 vector-based vaccines could elicit potent cellular immune responses that were not significantly suppressed by anti-Ad5 immunity (Barouch D H et al. 2004. J Immunol 172:6290). Similarly, chimpanzee adenoviruses have been shown to elicit immune responses that were minimally affected by anti-Ad5 immunity (Farina S F et al. 2001. J Virol 75:11603; Pinto A R et al. 2003. J Immunol 171:6774). It was recently demonstrated that neutralizing antibodies and CD8⁺ T lymphocyte responses both contribute to anti-Ad5 immunity, although Ad5-specific neutralizing antibodies appear to play the primary role (Sumida S M et al. 2004. J Virol 78:2666).

While the use of low-neutralized serotypes in vector-based vaccines appears to be a very useful approach, studies in mice have shown that Ad35 vector-based vaccines proved less immunogenic than Ad5 vector-based vaccines where there was no pre-existing Ad5-immunity present (Barouch et al. 2004). This effect between the two serotypes may be due to their difference in tropism (Ad5 infects liver cells very efficiently, B-group viruses such as Ad11 and Ad35 do not, but have a higher tropism for e.g., primary fibroblasts and synoviocytes). The effect may also be caused by the fact that subgroup B viruses use another cellular receptor for cell entry (CD46 rather than the Coxsackie virus and adenovirus receptor, CAR). The same may hold true for Ad11, which is also a low-neutralized serotype, but also a serotype from subgroup B.

In addition to the subgroup B- and C-based adenovirus vaccines, a vaccine composition comprising a recombinant, replication-defective adenovirus based on Ad49 has also been described (see U.S. patent application Ser. No. 11/140,418). Ad49 exhibits a low prevalence when samples taken from different parts of the world are examined.

Besides the different levels of immune response that are elicited by the different recombinant adenoviral vectors based on different serotypes, pre-existing immunity is also defined through other parameters, such as geographical distribution. In one part of the world a certain serotype may have infected a higher percentage of the human population than in other parts. This may influence the choice of vector used in certain areas, depending on the general percentage of humans that encountered a previous infection with the serotype of choice. For instance, it was found that there is a high prevalence of pre-existing immunity to Ad5 in human populations, particularly in sub-Saharan Africa, which is an area with an extremely high occurrence of HIV. Because HIV is one of the viruses that most likely can be counteracted by vaccination through adenoviral vectors, people in such areas would most benefit from adenoviruses that at least are not based on Ad5, and are more preferably based on a serotype that has a low prevalence in the area. For examples of differences in percentages in samples taken from different parts of the world, see WO 00/70071. Following the example from Africa, people that need vaccination there would most likely benefit most from adenoviruses that are not based on Ad5.

Clearly, there is a need in the field for alternative adenoviral vectors that do not encounter pre-existing immunities in the host, but that are still immunogenic and capable of inducing strong immune responses against the proteins encoded by the heterologous nucleic acids inserted in the nucleic acid carried by the vector. Preferred serotypes are those that encounter low neutralizing effects in human populations.

### BRIEF DESCRIPTION OF THE DRAWINGS

FIG. 1 shows the three—(FIG. 1A) or two-plasmid system (FIG. 1B) that is used to generate recombinant replication-defective adenoviruses in packaging cell, in this particular case: Ad48.

FIG. 2 shows the entire genome sequence of human adenovirus serotype 48 (Ad48); SEQ ID NO:2.

FIG. 3 shows the entire genome sequence of human adenovirus serotype 26 (Ad26); SEQ ID NO:1.

FIG. 4 shows the cellular immune response (CD8+ T cells) upon injection of Ad26—(FIG. 4A), Ad35—(FIG. 4B) or Ad48—(FIG. 4C) recombinant viral vectors comprising a SIVmac239 Gag antigen in mice up to 15 days after injection, and in comparison to Ad5, Ad11, and Ad49 recombinant viral vectors comprising the same antigen with different doses: 10⁹ vp, 10⁸ vp and 10⁷ vp for 30 days after injection (FIGS. 4D and 4E). Vaccine-elicited cellular immune responses were evaluated after pre-immunization with empty Ad5 vectors and subsequent injection of 10⁹ vp of the different vectors, assayed by D^{b}/AL11 tetramer binding assays (FIGS. 4D and 4F) and IFN-γ ELISPOT assays (FIGS. 4E and 4G).

FIG. 5 shows the cellular immune response in rhesus monkeys, 2 and 4 weeks after injection with recombinant Ad5, Ad26, Ad48 and Ad49 vectors carrying the Gag transgene.

FIG. 6 shows the cellular immune response upon heterologous prime/boost injections with the combinations indicated, using Ad35 as a priming vector (FIG. 6A), Ad26 as a priming vector (FIG. 6B), or with DNA priming (FIG. 6C). Boost injections were given at day 28.

### SUMMARY OF THE INVENTION

The present invention relates to an isolated nucleic acid having at least 90% sequence identity to the sequence set forth in SEQ ID NO:1 or SEQ ID NO:2, wherein said nucleic acid comprises structural and non-structural elements of Ad26 or Ad48 respectively. Preferably, said nucleic acid has a deletion in- or of the E1 region, said deletion rendering the nucleic acid substantially replication-defective, whereas it is even more preferred that said nucleic acid has a deletion in- or of the E3 region. Preferably, the nucleic acid further comprises a heterologous gene of interest in the deleted E1 region, under the control of a promoter. The gene of interest preferably encodes a viral protein, such as a protein of Human Immunodeficiency Virus (HIV).

The invention also relates to a recombinant replication-defective adenovirus based on Ad26 or Ad48, comprising a nucleic acid according to the invention. It furthermore relates to a two-plasmid system for generating the recombinant adenovirus in packaging cells. The invention also relates to a method of producing a recombinant adenovirus according to the invention in a packaging cell cultured in a suitable medium.

The invention furthermore relates to a pharmaceutical composition comprising a recombinant adenovirus according to the invention. As the recombinant adenovirus is preferably applied in therapy, the invention also relates to a recombinant replication-defective adenovirus according to the invention for use as a medicament. Such use may be in gene therapy, but preferably in vaccination programs.

The invention also relates to use of a recombinant replication-defective adenovirus according to the invention in the manufacture of a medicament for the therapeutic, prophylactic or diagnostic treatment of an infectious disease, such as AIDS, malaria, ebola-infections, and tuberculosis. The invention also relates to a method of treating a host in need of treatment or in need of vaccination, comprising administering to said host a recombinant replication-defective adenovirus or a pharmaceutical composition according to the invention.

### DETAILED DESCRIPTION

The present invention relates to two recombinant replication-deficient adenoviral vectors from subgroup D, namely serotype 26 (Ad26) and serotype 48 (Ad48). As outlined above, the presence of pre-existing immunity against commonly used Ad5 vectors negatively influences the effect of using vaccine compositions comprising viral vectors based on this serotype. Whereas Ad11 and Ad35 provide good alternatives for Ad5 (these vectors generally encounter pre-existing neutralizing activity in only a small percentage of human hosts), the fact that both serotypes are from subgroup B, means that prime/boost regimens using both Ad11 and Ad35 are not feasible due to a possible cross-neutralization (Lemckert A A et al. 2005 J Virol. 79:9694-9701). The subgroup D adenoviruses provide an excellent alternative to the subgroup B virus-based vaccines, such as Ad11 and Ad35, because the subgroup D adenoviruses also exhibit low pre-existing immunity in human hosts, especially in areas around the world where vaccination against life-threatening diseases such as AIDS are most required.

For both vectors, the genome was purified and the sequence of the genome and the genomic organization was completely identified. The entire genomic sequences were not previously disclosed in the art. After mapping the different regions within the genome, by comparison to other, known adenoviral genomes, the Ad26 and Ad48 genomes were manipulated such that the E1 region was removed. This enables the production of replication-deficient viruses. The deletion was such that the remaining sequences contained no overlap with sequences present in the packaging cells used to produce the recombinant vectors. Such systems to generate replication-defective adenoviruses are known in the art and have been applied to generate replication competent adenovirus (rca)-free batches based on Ad5, Ad11, Ad35 and Ad49 (see WO 97/00326, WO 00/70071; WO 02/40665; U.S. Ser. No. 11/140,418, all incorporated herein by reference).

To produce significant amounts of non-subgroup B adenoviruses on a packaging cell that expresses the E1 proteins of Ad5, which is a subgroup C adenovirus, it was found previously that either cell lines should be generated: that express the E1 proteins from a serotype from that particular subgroup (especially the E1B.55K protein; see WO 00/70071; WO 02/40665; U.S. Pat. No. 6,492,169; U.S. Pat. No. 6,869,794; U.S. Pat. No. 6,974,695); or that co-express E4orf6 of such a serotype (293.E4orf6 cells, see U.S. Pat. No. 6,127,175). In yet another technology, the E4orf6 from a subgroup C adenovirus replaces the E4orf6 region from the other subgroup serotype of interest such that it can be produced, free of rca, on Ad5-E1 transformed packaging cells (see WO 03/104467, incorporated herein by reference in its entirety).

The latter technology is also applied herein to produce the recombinant viruses based on Ad26 and Ad48: the E4orf6 region of the backbone genome of Ad26 and Ad48 is replaced by the corresponding region from Ad5, which enables one to produce replication-defective adenovirus batches on packaging cells, as the E4orf6 gene product is then compatible with the E1B-55K protein expressed in the packaging cell. The most preferred packaging cell that is used for producing Ad26- and Ad48-based recombinant adenovirus batches that are substantially free from rca, is the PER.C6® cell line, represented by the cells deposited at the European Collection of Cell Cultures (ECACC, Porton Down, Wiltshire, SP4 0JG, UK) under no. 96022940. It is to be understood that the invention is by no means limited to the use of the Ad5-E4orf6 region or the use of packaging cells that only express E1 from Ad5. This is just one technology available that enables one to produce recombinant adenoviruses, albeit a very convenient way since the necessity to generate separate cell lines is circumvented.

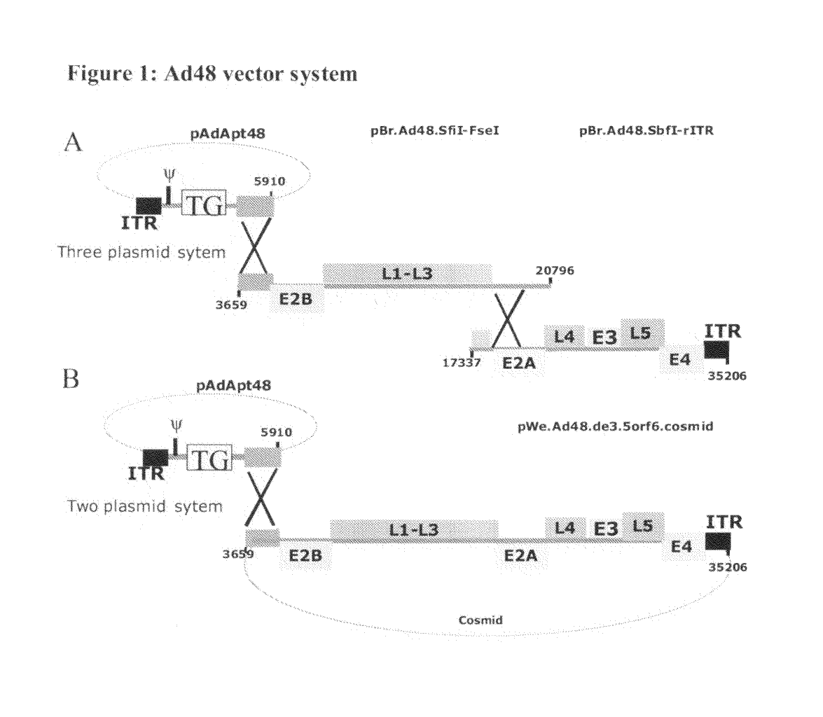

Although numerous systems exist for producing recombinant adenoviruses, the present invention makes use of the so-called two-plasmid system, in which one plasmid, generally referred to as the adapter plasmid, comprises part of the left side of the genome, including the left-end Inverted Terminal Repeat (left ITR), packaging signal, etc., and the functional deletion of the E1 region, whereas another plasmid (also referred to as a cosmid) comprises most of the right part of the adenoviral genome, including the E4orf6 swap discussed above. The same system can also be used in a three (or more plasmid system), as long as homologous recombination can occur between the different plasmids. The system is depicted in FIG. 1. The adapter plasmid and the cosmid both comprise a sequence of overlap, which enables one to generate a full-length adenoviral genome with all the characteristics of the separate plasmids. The adapter plasmid generally comprises an expression cassette at the position of the E1 region, wherein the expression cassette typically comprises a promoter that stimulates expression of a cloned transgene, and furthermore a poly-adenylation signal. The E1 region of the recombinant adenovirus is deleted, either partially or completely, such that there is no overlap with the E1 region present in the packaging cell line, thereby circumventing the generation of rca. The cosmid typically includes the E4orf6 swap as outlined above and preferably lacks most if not all of the E3 region, which is not required for replication and packaging of the adenoviral particle. Deletion of the E3 region is generally preferred if large transgene sequences are to be incorporated into the cosmid since the genome size which can be packaged into a functional particle is limited to approximately 105% of the wild type size. Although not applied herein, it is to be understood that other modifications may be introduced in the adenoviral genome, such as deletion of the E2A region, or most if not the entire E4 region. The packaging cell can complement these deficiencies by delivering the functionality of the E2A region by, for instance, a temperature sensitive E2A mutant, or by delivering the E4 functions, such as in 293-E4orf6 cells, as discussed above. All such systems are known in the art and such modifications of the adenoviral genomes are within the scope of the present invention, which in principal relates to the two novel Ad26 and Ad48 genomic sequences, and the use thereof.

### Ad26

The present invention relates to an isolated nucleic acid having at least 90% sequence identity to the sequence set forth in SEQ ID NO:1, wherein said nucleic acid comprises structural and non-structural elements of an adenovirus serotype 26 (Ad26). More preferably, the isolated nucleic acid has 95% sequence identity, and even more preferably, 98-99% sequence identity. Most preferred is an embodiment in which the sequence is identical to that shown is SEQ ID NO:1. ‘Structural elements’ as used herein refers to genes encoding adenoviral proteins that are a physical part of the adenoviral particle. Important (in terms of immunogenicity and in terms of building blocks) structural elements are genes that encode the fiber, the hexon and the penton proteins, found in the capsid of the virus. ‘Non-structural elements’ as used herein refers to genes that encode proteins and gene products that do not form part of the viral physical particle but that are involved in replication, transcription and packaging of the genome into particles. Examples are the early genes E1, E2, E3 and E4.

As shown in the examples, sequence differences (silent and coding) may be found in different isolates of the same serotype. Such differences are also part of the present invention. In a preferred embodiment, the invention relates to an isolated nucleic acid according to the invention, wherein said nucleic acid has a deletion in- or of the E1 region, said deletion rendering the nucleic acid substantially replication-defective. The art is clear about how to make adenoviruses replication-deficient (of replication-defective) by altering the E1 region. If the proteins encoded by the E1 region are no longer expressed, the virus can no longer express the other early genes, and late genes from its genome. Many of such genes and hence, their encoded proteins are required for replication, and subsequent packaging of the replicated genomes into viral particles. When the E1 region is mutated in a functional manner, such replication and packaging can no longer take place. The mutation of the E1 region can be made in different ways, either through point mutations, where the open reading frame is interrupted (of one or more of the encoded proteins), or where most if not the entire E1 region is deleted from the genome. The latter is preferred as it renders the genome replication defective (no E1 proteins are transcribed), it allows the integration of a heterologous gene in an expression cassette and it prevents overlap with E1 regions present in packaging cells. It is to be understood that the sequence identity of at least 90%, and more preferably 95%, even more preferably 98-99% and most preferably 100% counts for the sequence left after the functional mutation of the E1 region. So, if most, if not the entire region of E1 (nt. 472-3365) has been deleted, the sequence identity of the remaining sequence of SEQ ID NO:1 should still at least be 90% to be within the scope of what is claimed.

In another preferred embodiment, the invention relates to an isolated nucleic acid according to the invention, wherein said nucleic acid has a deletion in- or of the E3 region. The E3 region in Ad26 is located on nt. 26692-30682. The E3 region is preferably deleted, as it is not required for replication and packaging (since it is for instance involved in suppressing host immune responses after viral infection). It allows space to clone transgenes that are otherwise too big and that otherwise would render the entire sequence too large to be packaged. Again, as indicated above, it is noted that the when E3 is deleted (with or without the E1 deletion), the remaining sequence would still have to be at least 90% identical to the sequence of SEQ ID NO:1 to fall within the scope of what is being claimed.

In another preferred embodiment, the invention relates to an isolated nucleic acid according to the invention, wherein said nucleic acid further comprises a sequence encoding the E4orf6 gene product of an adenovirus of subgroup C. The inclusion of the E4orf6 (and part of the E4orf6/7 region as outlined in the examples) allows the expression of the recombinant (subgroup D) virus on packaging cells that have been transformed and immortalized by E1 from a subgroup C virus. As PER.C6® and 293 cells have been transformed by E1 from Ad5, it is most preferred that the subgroup C adenovirus from which this E4 region is derived, is Ad5. However, also other subgroup C adenoviruses may deliver their E4orf6 region as long as it compatible with the E1-55K protein from Ad5 (as extensively discussed in WO 03/104467). More preferably, the Ad26 E4orf6 region (located between nt. 32166-33248) is removed and replaced by the equivalent Ad5 E4orf6 coding sequence. This would allow more space.

In one embodiment of the present invention, the isolated nucleic acid according to the invention, further comprises a heterologous gene of interest. Heterologous means that it is non-adenoviral. The heterologous gene of interest may be a viral transgene, a bacterial transgene, a transgene from a parasite, a human transgene, an animal transgene, or a synthetic transgene. If required, the gene of interest is codon-optimized to ensure proper expression in the treated host. Codon-optimization is a technology widely applied in the art. Preferably, said heterologous gene of interest is cloned into the region of the E1 deletion. In another aspect of the invention, said heterologous gene of interest is under the control of a promoter. The promoter may be adenovirus-derived (one example is the Major Late Promoter), but preferably, the promoter is heterologous. Examples of preferred heterologous promoters that are used for the expression of the transgene are the CMV promoter and the RSV promoter. Preferably, the promoter is located upstream of the gene of interest within the expression cassette. In one preferred aspect of the invention, the invention relates to an isolated nucleic acid, wherein said heterologous gene of interest encodes a protein selected from the group consisting of: a viral protein, an antigenic determinant of a non-viral pathogenic organism (e.g., a bacterium, a protozoan, a fungus, or a parasite), a tumor-specific antigen, a human protein, and a cytokine. Preferably, said heterologous gene of interest encodes a viral protein, wherein said viral protein elicits an immune response in a host. Preferably, the viral protein is a protein of Human Immunodeficiency Virus (HIV), Alphavirus, Arbovirus (i.e., yellow fever virus), Borna Disease Virus, Bunyavirus, Calicivirus, Varicella Zoster Virus, Coronavirus (e.g., SARS Virus), Coxsackievirus, Cytomegalovirus, Flavivirus, Epstein-Barr Virus, Hantavirus, Hepatitis Virus (e.g., Hepatitis B or C Virus), Herpes Simplex Virus (1 or 2), Rhabdovirus (i.e., rabies virus), Influenza Virus, Paramyxovirus (i.e., parainfluenza virus 1 or 3), Rubulavirus (i.e., mumps virus), Morbillavirus (i.e., measles virus), Poliovirus, Pneumovirus (i.e., Human respiratory syncytial virus), Polyomavirus, Rotavirus, Rift Valley Fever Virus, Rubella Virus, Smallpox Virus, or West Nile Fever Virus. More preferably, said viral protein is a protein of Human Immunodeficiency Virus (HIV). Examples of HIV-derived proteins that may be expressed from the gene of interest are gag, env, and pol. The application of HIV-derived antigens that may be used in the context of a recombinant adenovirus have been described in the art (Shiver J W et al. 2002. Nature 415:331-335; Casimiro D R et al. 2003. J Virol 77:6305-6313). The heterologous gene of interest can also encode an antigenic determinant of a pathogenic organism, such as a bacterium, a protozoan, a fungus, and a parasite, wherein said an antigenic determinant elicits an immune response in a host. Preferably, the antigenic determinant is or is derived from a protein from Escherichia coli, Mycobacterium tuberculosis, Bacillus anthracis, Salmonella, Staphylococcus aureus, Streptococcus pneumoniae, Streptococcus pyogenes, Helicobacter pylori, Francisella tularensis, Cryptosporidium, Giardia lamblia, Plasmodium, Trypanosoma cruzi, Pneumocystis jiroveci, Tinea, Candida, a roundworm, Sarcoptes scabiei, a tapeworm, or a flatworm.

The invention relates to a recombinant replication-defective adenovirus based on Ad26, comprising a nucleic acid according to the invention. Preferably, said nucleic acid has a sequence that is at least 90% identical to the sequence of SEQ ID NO:1, and more preferred is a sequence that is at least 90% identical to the sequence of SEQ ID NO:1 that remains after deletion of the E1 and/or the E3 region and/or wherein the E4orf6 region has been replaced by the E4orf6 region of a subgroup C adenovirus, more preferably that of Ad5.

The invention also relates to a system by which the recombinant replication-defective adenoviruses according to the invention can be produced. Hence, the invention also relates to a two-plasmid system, together comprising a nucleic acid according to the invention, wherein said two plasmids each contain part of the entire sequence including an overlapping, sequence, which allows homologous recombination between said two plasmids resulting in a full length nucleic acid. It is to be understood that such production system can also be applied by using more than two plasmids, wherein there is a requirement of more homologous recombination events. To allow the most efficient production of full length genomes, it is preferred to use the two-plasmid system (in which one plasmid is an adapter plasmid and the other plasmid, containing most of the adenoviral genome, is generally referred to as the cosmid).

The invention also relates to a method of producing a recombinant adenovirus according to the invention, comprising culturing packaging cells in a suitable medium; transfecting said packaging cells with an isolated nucleic acid according to the invention; allowing replication of said nucleic acid in said packaging cells; and harvesting produced recombinant adenovirus from said medium and/or said cells. Methods of transfecting plasmids (and cosmids) are well known in the art. Moreover, suitable medium for packaging cells have also been described in the art and are not elaborated on herein. Harvesting methods are also known to the skilled person.

The invention also relates to a pharmaceutical composition comprising a recombinant adenovirus according to the invention, and a suitable excipient. Suitable excipients are compounds that are allowed to be included in pharmaceutical compositions for human and/or animal use. They comprise suitable carriers such as water, and buffered solutions, generally comprising salts and/or detergents.

In another aspect of the invention, the invention relates to a recombinant replication-defective adenovirus according to the invention for use as a medicament.

In yet another aspect of the invention, the invention relates to the use of a recombinant replication-defective adenovirus according to the invention in the manufacture of a medicament for the therapeutic, prophylactic or diagnostic treatment of an infectious disease. Preferably, in such use, said infectious disease is selected from the group consisting of: AIDS, malaria, ebola-infections, and tuberculosis.

The invention also relates to a method of treating a host in need of treatment or in need of vaccination, comprising administering to said host a recombinant replication-defective adenovirus according to the invention, or a pharmaceutical composition according to the invention. Preferably said vaccination is against diseases as AIDS, malaria, ebola-infections, or tuberculosis.

### Ad48

The present invention also relates to an isolated nucleic acid having at least 90% sequence identity to the sequence set forth in SEQ ID NO:2, wherein said nucleic acid comprises structural and non-structural elements of an adenovirus serotype 48 (Ad48). More preferably, the isolated nucleic acid has 95% sequence identity, and even more preferably, 98-99% sequence identity. Most preferred is an embodiment in which the sequence is identical to that shown is SEQ ID NO:2. ‘Structural elements’ as used herein refers to genes encoding adenoviral proteins that are a physical part of the adenoviral particle. Important (in terms of immunogenicity and in terms of building blocks) structural elements are genes that encode the fiber, the hexon and the penton proteins, found in the capsid of the virus. ‘Non-structural elements’ as used herein refers to genes that encode proteins and gene products that do not form part of the viral physical particle but that are involved in replication, transcription and packaging of the genome into particles. Examples are the early genes E1, E2, E3 and E4.

As shown in the examples, sequence differences (silent and coding) may be found in different isolates of one serotype. Such differences are also part of the present invention. In a preferred embodiment, the invention relates to an isolated nucleic acid according to the invention, wherein said nucleic acid has a deletion in- or of the E1 region, said deletion rendering the nucleic acid substantially replication-defective. The art is clear about how to make adenoviruses replication-deficient (of replication-defective) by altering the E1 region. If the proteins encoded by the E1 region are no longer expressed, the virus can no longer express the other early genes, and late genes from its genome. Many of such genes and hence, their encoded proteins are required for replication, and subsequent packaging of the replicated genomes into viral particles. When the E1 region is mutated in a functional manner, such replication and packaging can no longer take place. The mutation of the E1 region can be made in different ways, either through point mutations, where the open reading frame is interrupted (of one or more of the encoded proteins), or where most if not the entire E1 region is deleted from the genome. The latter is preferred as it renders the genome replication defective (no E1 proteins are transcribed), it allows the integration of a heterologous gene in an expression cassette and it prevents overlap with E1 regions present in packaging cells. It is to be understood that the sequence identity of at least 90%, and more preferably 95%, even more preferably 98-99% and most preferably 100% counts for the sequence left after the functional mutation of the E1 region. So, if most, if not the entire region of E1 (nt. 463-3361) has been deleted, the sequence identity of the remaining sequence of SEQ ID NO:2 should still at least be 90% to be within the scope of what is claimed. In another preferred embodiment, the invention relates to an isolated nucleic acid according to the invention, wherein said nucleic acid has a deletion in- or of the E3 region. The E3 region of Ad48 is located on nt. 26656-30735. The E3 region is preferably deleted, as it is not required for replication and packaging (since it is for instance involved in suppressing host immune responses after viral infection). It allows space to clone transgenes that are otherwise too big and that otherwise would render the entire sequence too large to be packaged. Again, as indicated above, it is noted that the when E3 is deleted (with or without the E1 deletion), the remaining sequence would still have to be at least 90% identical to the sequence of SEQ ID NO:2 to fall within the scope of what is being claimed.

In another preferred embodiment, the invention relates to an isolated nucleic acid according to the invention, wherein said nucleic acid further comprises a sequence encoding the E4orf6 gene product of an adenovirus of subgroup C. The inclusion of the E4orf6 (and part of the E4orf6/7 region as outlined in the examples) allows the expression of the recombinant (subgroup D) virus on packaging cells that have been transformed and immortalized by E1 from a subgroup C virus. As PER.C6® and 293 cells have been transformed by E1 from Ad5, it is most preferred that the subgroup C adenovirus from which this E4 region is derived, is Ad5. However, also other subgroup C adenoviruses may deliver their E4orf6 region as long as it compatible with the E1-55K protein from Ad5 (as extensively discussed in WO 03/104467). More preferably, the Ad48 E4orf6 region (located between nt. 32241-33291) is removed and replaced by the equivalent Ad5 E4orf6 coding sequence. This would allow more space.

In one embodiment of the present invention, the isolated nucleic acid according to the invention, further comprises a heterologous gene of interest. Heterologous means that it is non-adenoviral. The heterologous gene of interest may be a viral transgene, a bacterial transgene, a transgene from a parasite, a human transgene, an animal transgene, or a synthetic transgene. If required, the gene of interest is codon-optimized to ensure proper expression in the treated host. Codon-optimization is a technology widely applied in the art. Preferably, said heterologous gene of interest is cloned into the region of the E1 deletion. In another aspect of the invention, said heterologous gene of interest is under the control of a promoter. The promoter may be adenovirus-derived (one example is the Major Late Promoter), but preferably, the promoter is heterologous. Examples of preferred heterologous promoters that are used for the expression of the transgene are the CMV promoter and the RSV promoter. Preferably, the promoter is located upstream of the gene of interest within the expression cassette.

In one preferred aspect of the invention, the invention relates to an isolated nucleic acid, wherein said heterologous gene of interest encodes a protein selected from the group consisting of: a viral protein, an antigenic determinant of a non-viral pathogenic organism (e.g., a bacterium, a protozoan, a fungus, or a parasite), a tumor-specific antigen, a human protein, and a cytokine. Preferably, said heterologous gene of interest encodes a viral protein, wherein said viral protein elicits an immune response in a host. Preferably, the viral protein is a protein of Human Immunodeficiency Virus (HIV), Alphavirus, Arbovirus (i.e., yellow fever virus), Borna Disease Virus, Bunyavirus, Calicivirus, Varicella Zoster Virus, Coronavirus (e.g., SARS Virus), Coxsackievirus, Cytomegalovirus, Flavivirus, Epstein-Barr Virus, Hantavirus, Hepatitis Virus (e.g., Hepatitis B or C Virus), Herpes Simplex Virus (1 or 2), Rhabdovirus (i.e., rabies virus), Influenza Virus, Paramyxovirus (i.e., parainfluenza virus 1 or 3), Rubulavirus (i.e., mumps virus), Morbillavirus (i.e., measles virus), Poliovirus, Pneumovirus (i.e., Human respiratory syncytial virus), Polyomavirus, Rotavirus, Rift Valley Fever Virus, Rubella Virus, Smallpox Virus, or West Nile Fever Virus. More preferably, said viral protein is a protein of Human Immunodeficiency Virus (HIV). Examples of HIV-derived proteins that may be expressed from the gene of interest are gag, env, and pol. The application of HIV-derived antigens that may be used in the context of a recombinant adenovirus have been described in the art (Shiver J W et al. 2002. Nature 415:331-335; Casimiro D R et al. 2003. J Virol 77:6305-6313). The heterologous gene of interest can also encode an antigenic determinant of a pathogenic organism (e.g., a bacterium, a protozoan, a fungus, and a parasite), wherein said an antigenic determinant elicits an immune response in a host. Preferably, the antigenic determinant is or is derived from a protein from Escherichia coli, Mycobacterium tuberculosis, Bacillus anthracis, Salmonella, Staphylococcus aureus, Streptococcus pneumoniae, Streptococcus pyogenes, Helicobacter pylori, Francisella tularensis, Cryptosporidium, Giardia lamblia, Plasmodium, Trypanosoma cruzi, Pneumocystis jiroveci, Tinea, Candida, a roundworm, Sarcoptes scabiei, a tapeworm, or a flatworm.

The invention relates to a recombinant replication-defective adenovirus based on Ad48, comprising a nucleic acid according to the invention. Preferably, said nucleic acid has a sequence that is at least 90% identical to the sequence of SEQ ID NO:2, and more preferred is a sequence that is at least 90% identical to the sequence of SEQ ID NO:2 that remains after deletion of the E1 and/or the E3 region and/or wherein the E4orf6 region has been replaced by the E4orf6 region of a subgroup C adenovirus, more preferably that of Ad5.

The invention also relates to a system by which the recombinant replication-defective adenoviruses according to the invention can be produced. Hence, the invention also relates to a two-plasmid system, together comprising a nucleic acid according to the invention, wherein said two plasmids each contain part of the entire sequence including an overlapping sequence, which allows homologous recombination between said two plasmids resulting in a full length nucleic acid. It is to be understood that such production system can also be applied by using more than two plasmids, wherein there is a requirement of more homologous recombination events. To allow the most efficient production of full-length genomes, it is preferred to use the two-plasmid system (in which one plasmid is an adapter plasmid and the other plasmid, containing most of the adenoviral genome, is generally referred to as the cosmid).

The invention also relates to a method of producing a recombinant adenovirus according to the invention, comprising culturing packaging cells in a suitable medium; transfecting said packaging cells with an isolated nucleic acid according to the invention; allowing replication of said nucleic acid in said packaging cells; and harvesting produced recombinant adenovirus from said medium and/or said cells. Methods of transfecting plasmids (and cosmids) are well known in the art. Moreover, suitable medium for packaging cells have also been described in the art and are not elaborated on herein. Harvesting methods are also known to the skilled person.

The invention also relates to a pharmaceutical composition comprising a recombinant adenovirus according to the invention, and a suitable excipient. Suitable excipients are compounds that are allowed to be included in pharmaceutical compositions for human and/or animal use. They comprise suitable carriers such as water, and buffered solutions, generally comprising salts and/or detergents.

In another aspect of the invention, the invention relates to a recombinant replication-defective adenovirus according to the invention for use as a medicament.

In yet another aspect of the invention, the invention relates to the use of a recombinant replication-defective adenovirus according to the invention in the manufacture of a medicament for the therapeutic, prophylactic or diagnostic treatment of an infectious disease. Preferably, in such use, said infectious disease is selected from the group consisting of: AIDS, malaria, ebola-infections, and tuberculosis. The invention also relates to a method of treating a host in need of treatment or in need of vaccination, comprising administering to said host a recombinant replication-defective adenovirus according to the invention, or a pharmaceutical composition according to the invention. Preferably said vaccination is against diseases as AIDS, malaria, ebola-infections, or tuberculosis.

It is known from the art, that viruses can be produced from so-called ‘minimal vectors’. These are vectors that have only the sequences of the viral genome that allows replication, such as the right and left Inverted Terminal Repeats and the packaging signal located generally at the 5′ end of the genome. Such minimal vectors also comprise an expression cassette including a promoter and a gene of interest, alike the expression cassette according to the present invention. To produce such minimal vectors and to have them packaged into a functional gene delivery vehicle, with the capsid of the preferred serotype, these vectors are generally replicated with the help of helper vectors or helper viruses that complement all the required elements missing from the minimal vector. The helper vectors provide the capsid proteins and may also provide all elements necessary for replication, transcription, and packaging. It is to be understood, that if a minimal vector is being made to produce a recombinant replication-defective adenovirus based on Ad26 or Ad48, this would also fall within the scope of the present invention as all the required sequences that should be present in such minimal vectors are herewith provided. The person skilled in the art will be able, using the knowledge of the genomic structure of known adenoviruses and the knowledge of making minimal vectors, and using the information provided herein to make and produce minimal vectors based on Ad26 and Ad48. So, the present invention also relates to minimal vectors, based on Ad26 and Ad48 (from the genome sequences provided in SEQ ID NO:1 and 2 respectively), from which all non-required nucleic acid sequences (non-required as in the sense of making a minimal vector) have been deleted. Then still, the remaining sequence has at least to be 90% identical to what is left to fall within the scope of what is claimed herein.

### EXAMPLES

The practice of this invention will employ, unless otherwise indicated, conventional techniques of molecular biology, cell biology, and recombinant DNA, which are within the skill of the person skilled in the art (see e.g. Sambrook, Fritsch and Maniatis, Molecular Cloning: A Laboratory Manual, 2^{nd} edition, 1989; Current Protocols in Molecular Biology, Ausubel F M, et al, eds, 1987; the series Methods in Enzymology (Academic Press, Inc.); PCR2: A Practical Approach, MacPherson M J, Hams B D, Taylor G R, eds, 1995).

### Example 1

### Sequence of Human Adenovirus Serotype 48

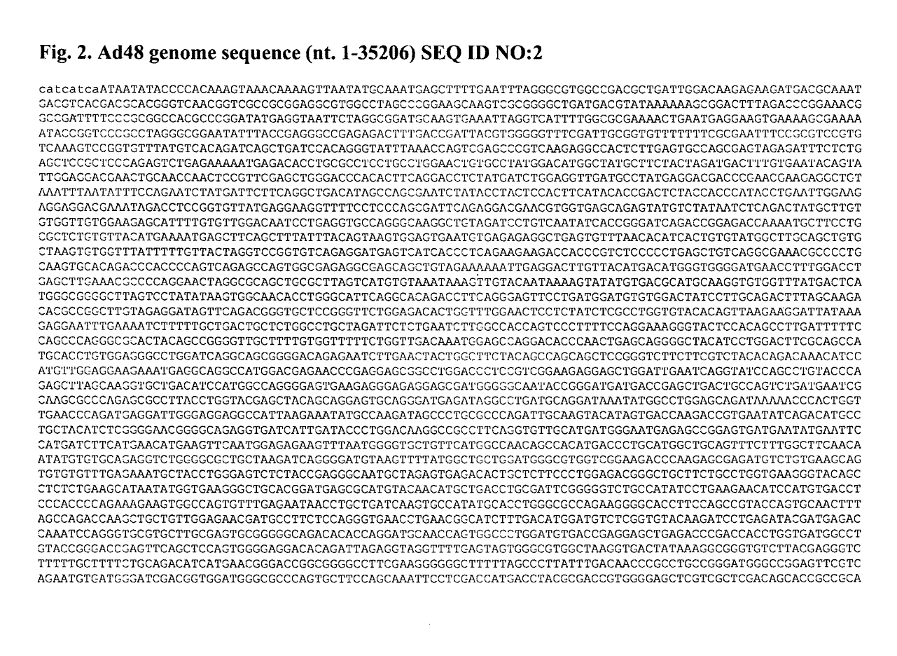

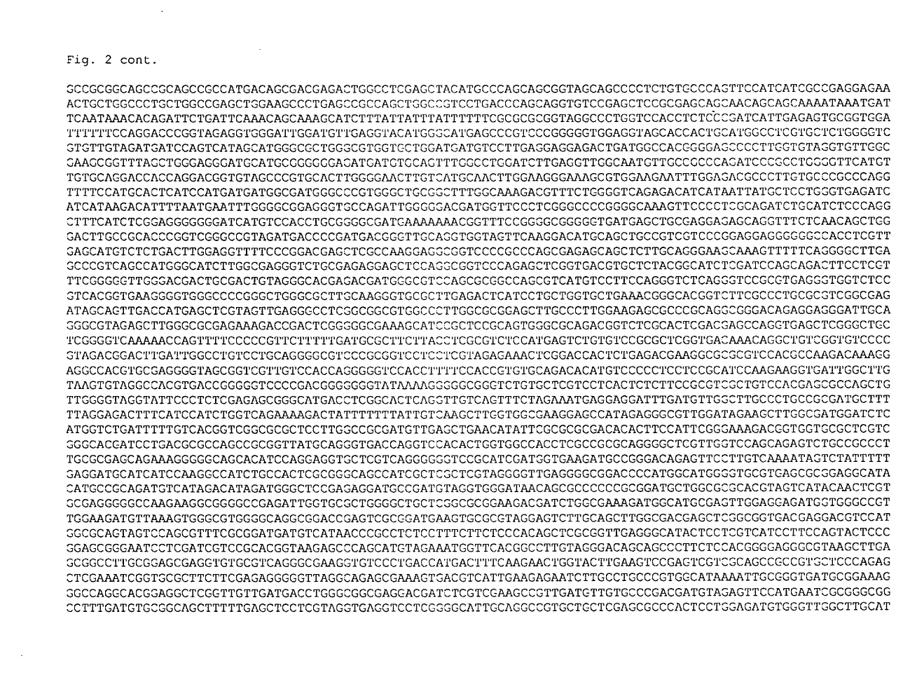

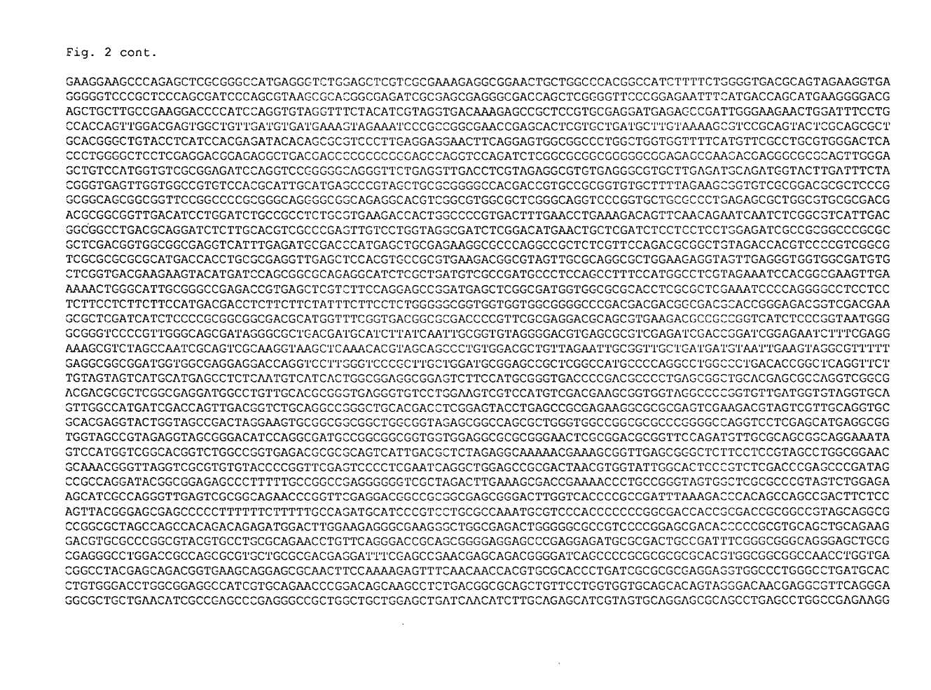

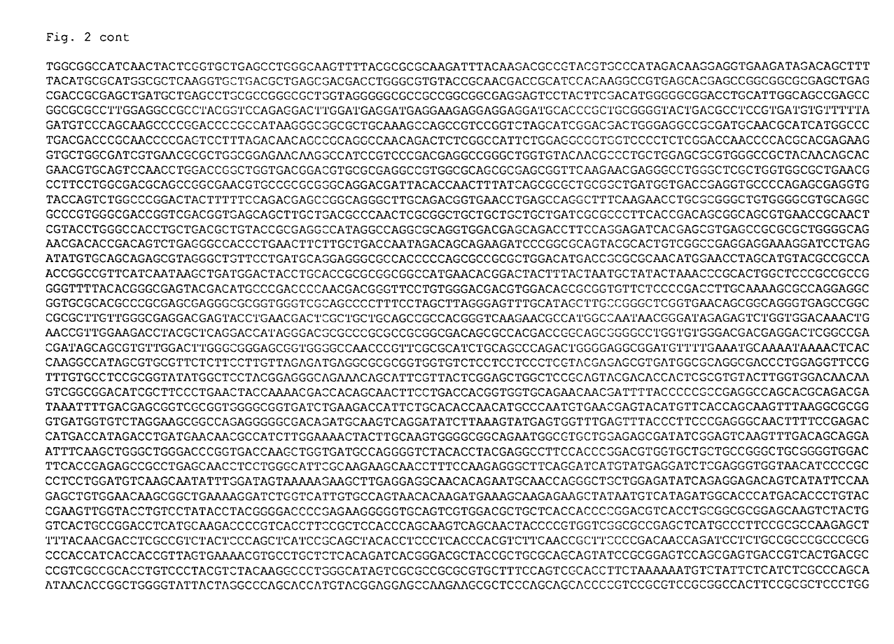

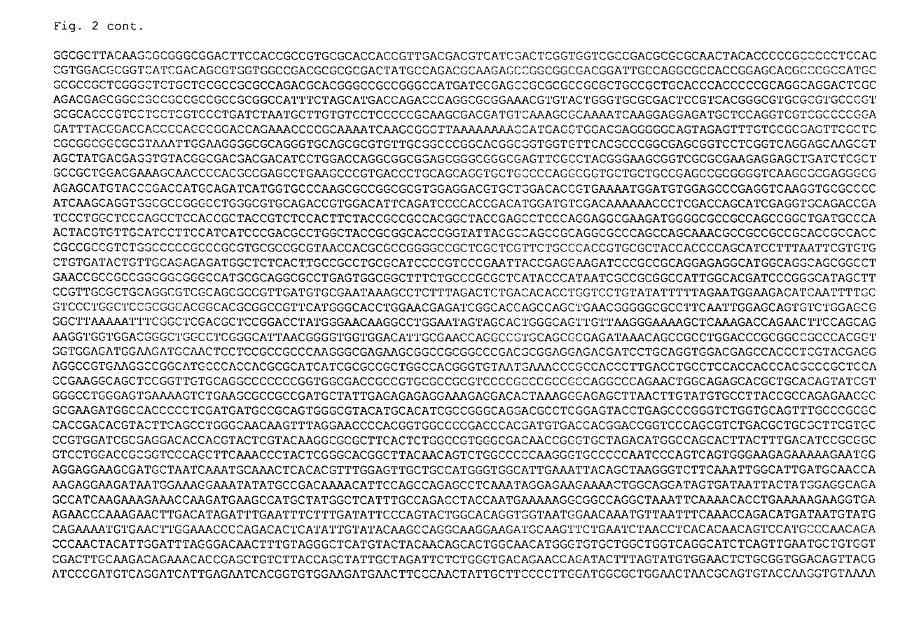

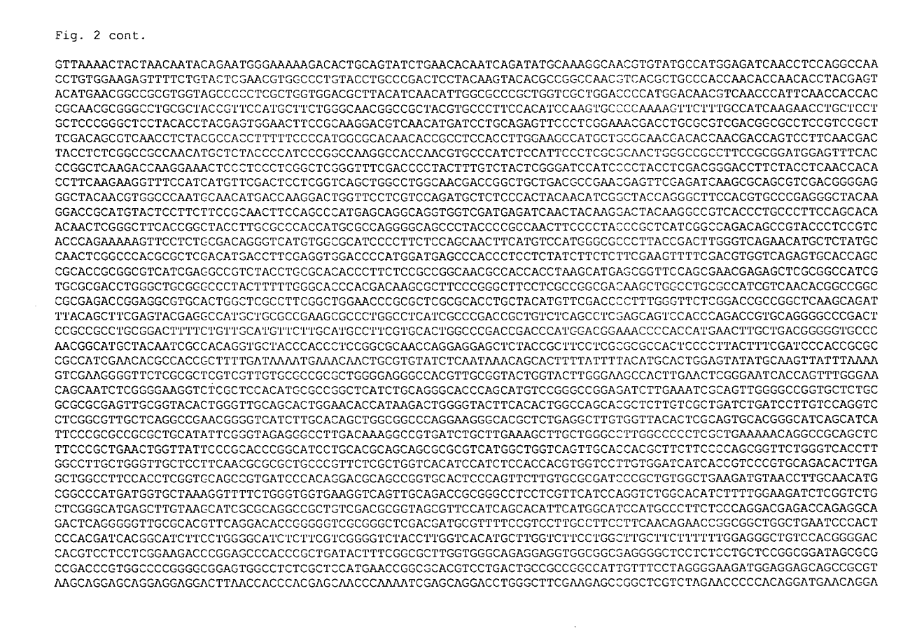

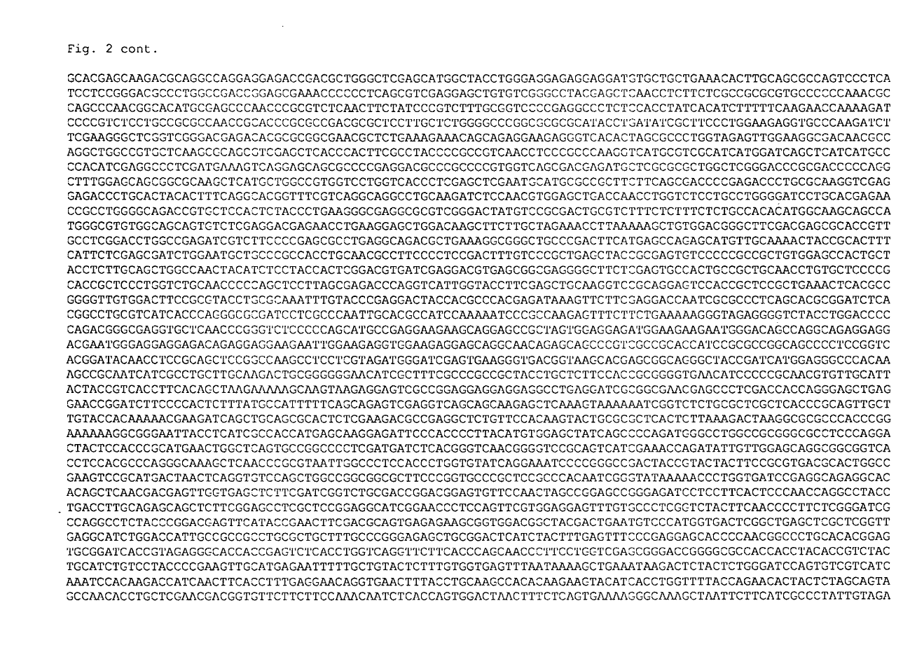

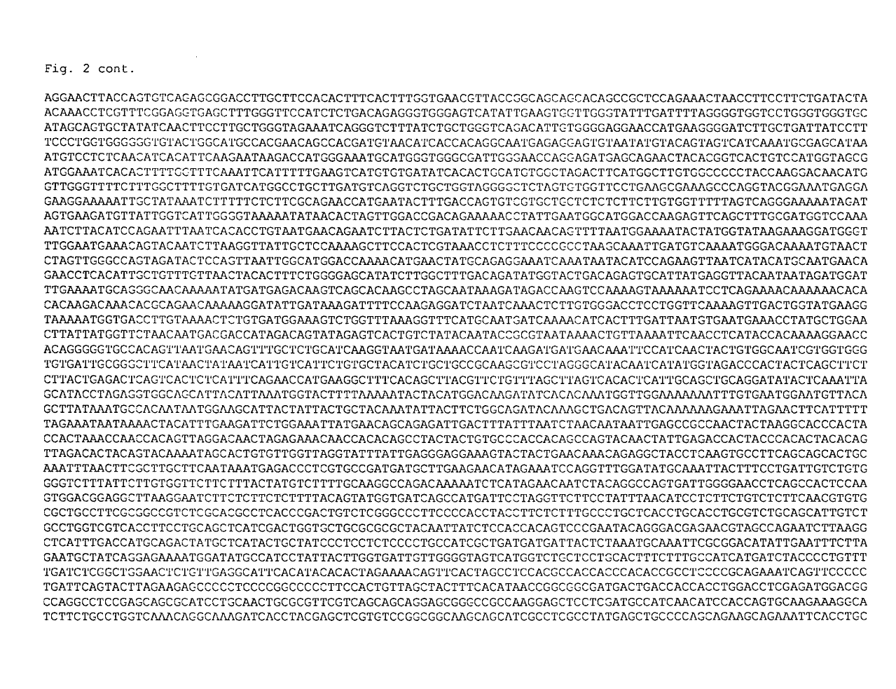

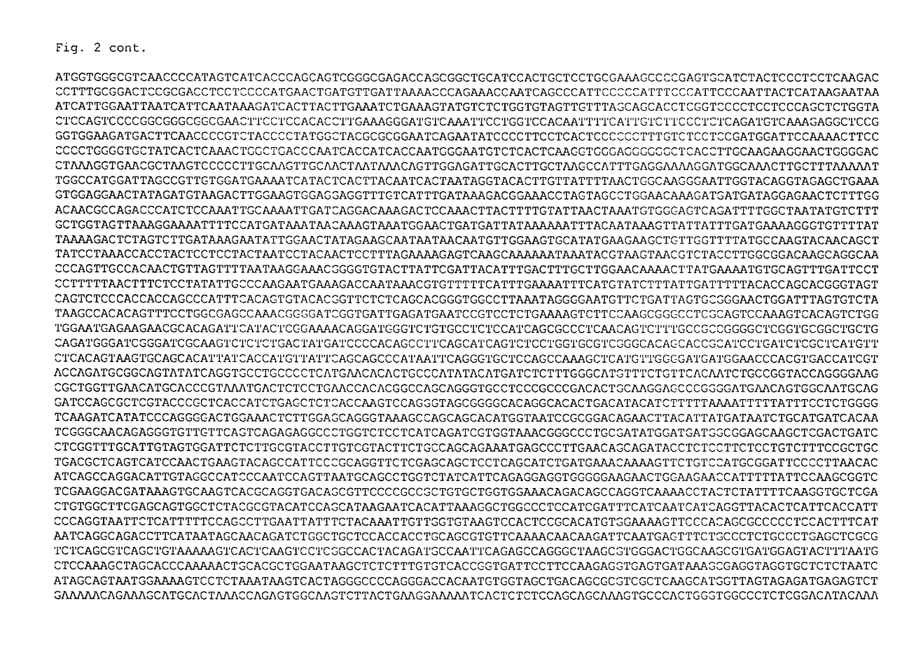

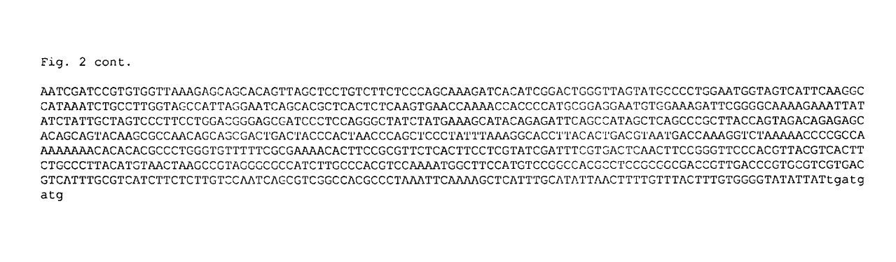

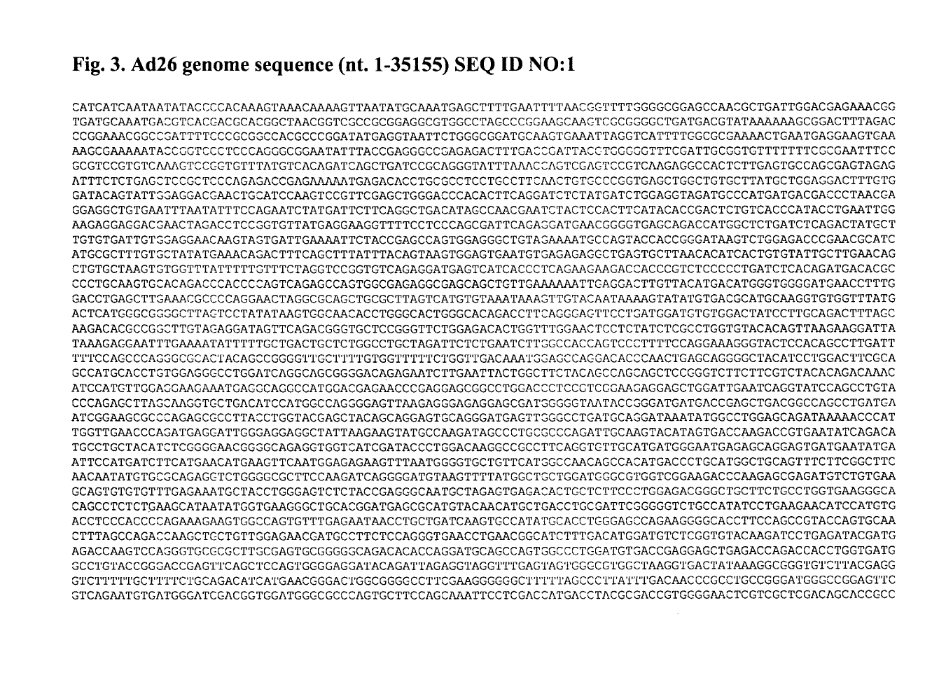

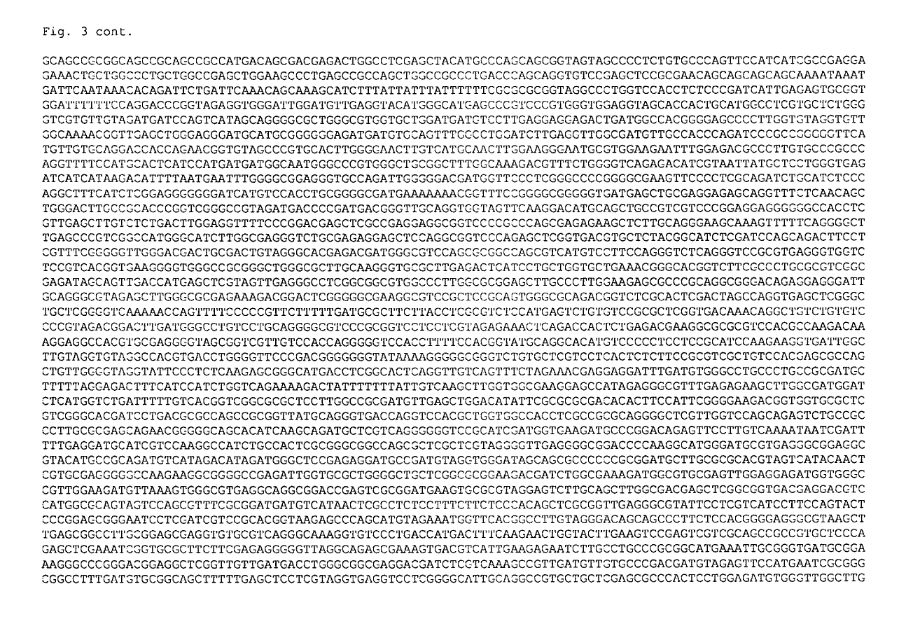

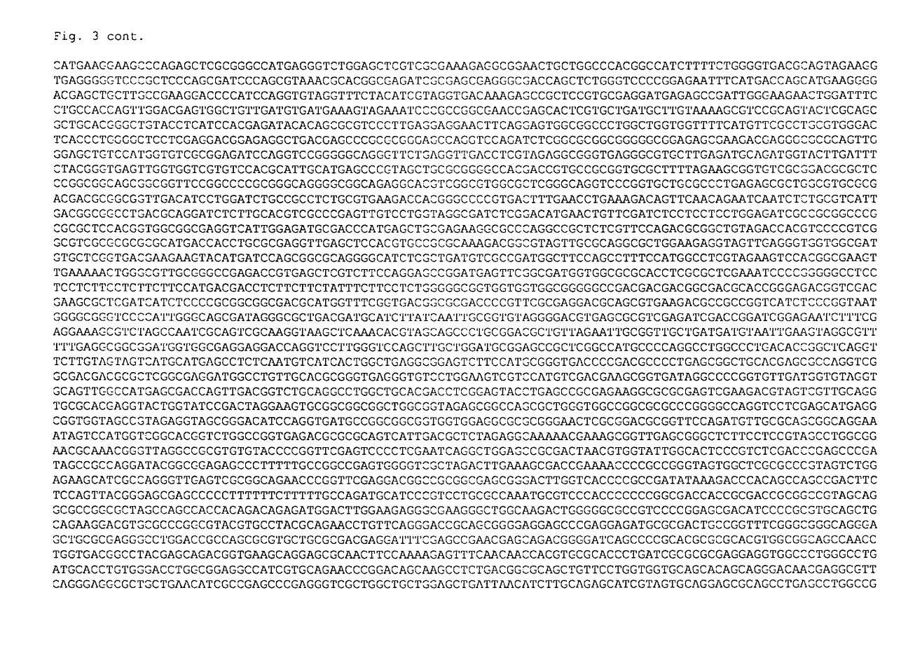

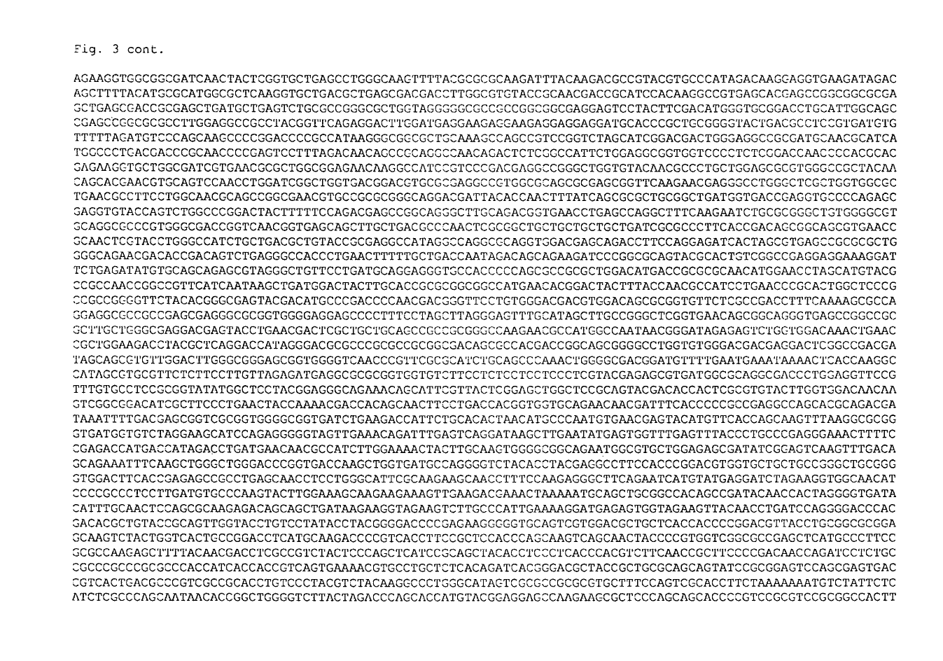

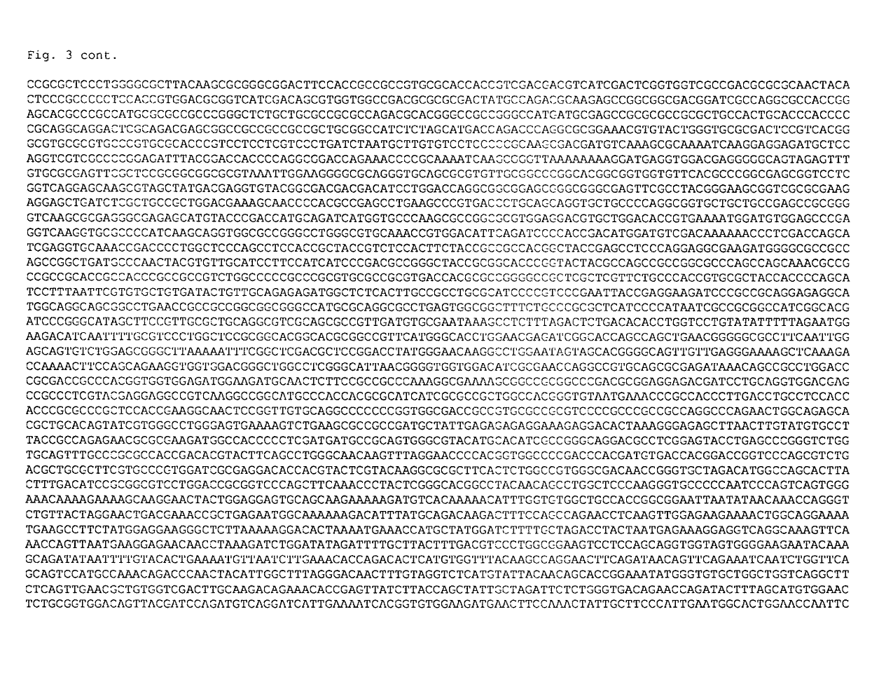

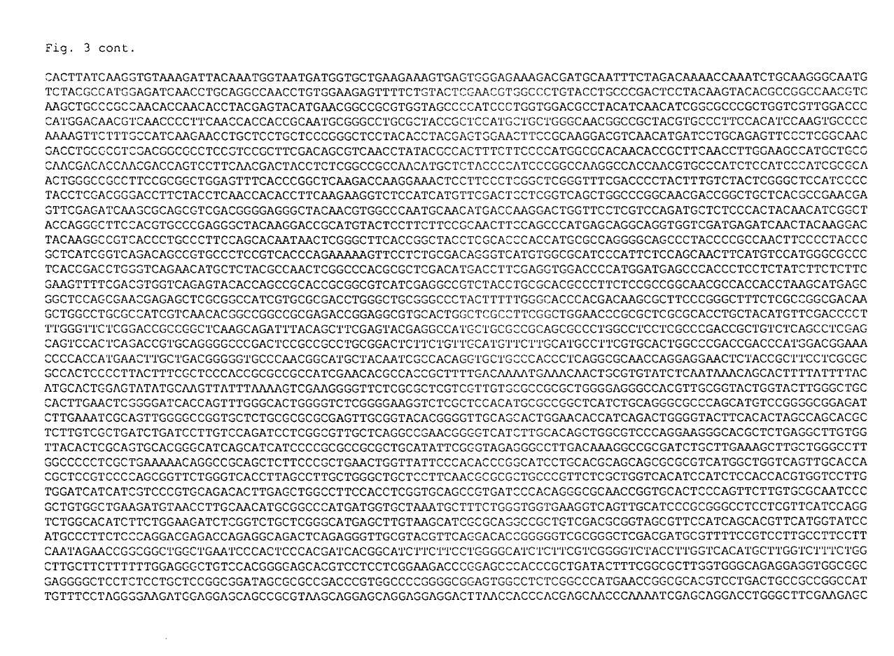

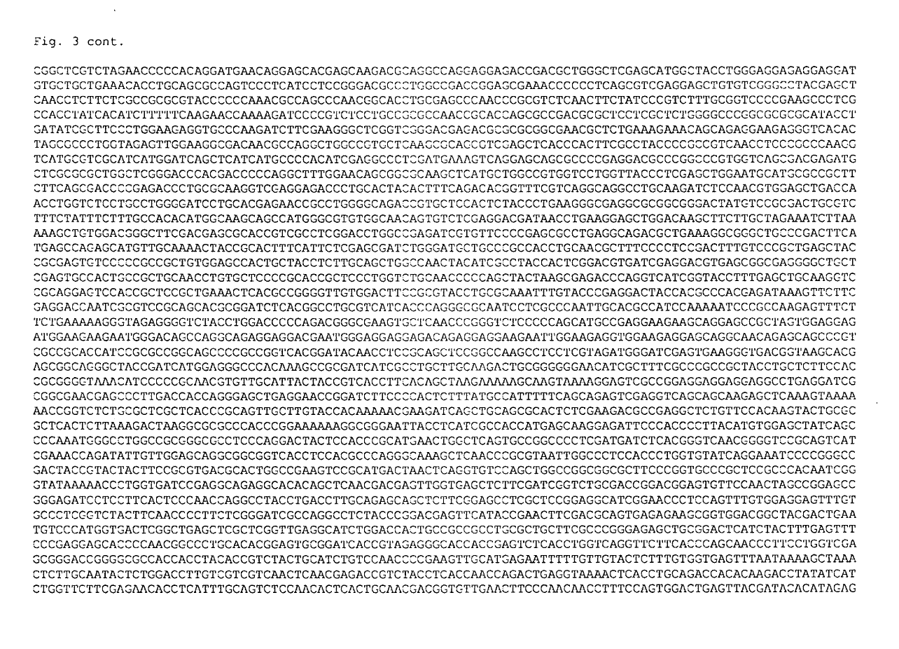

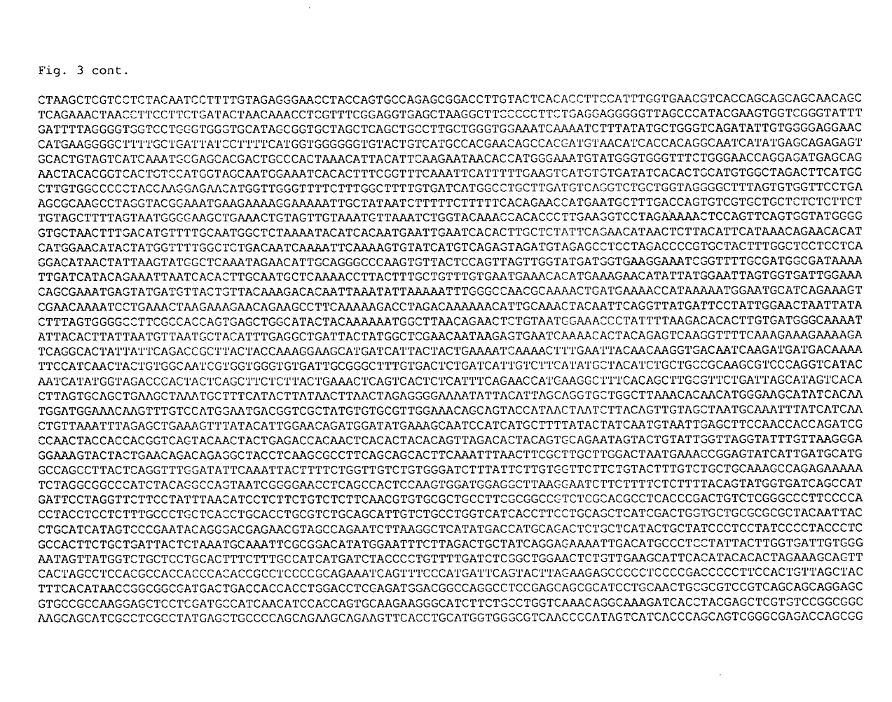

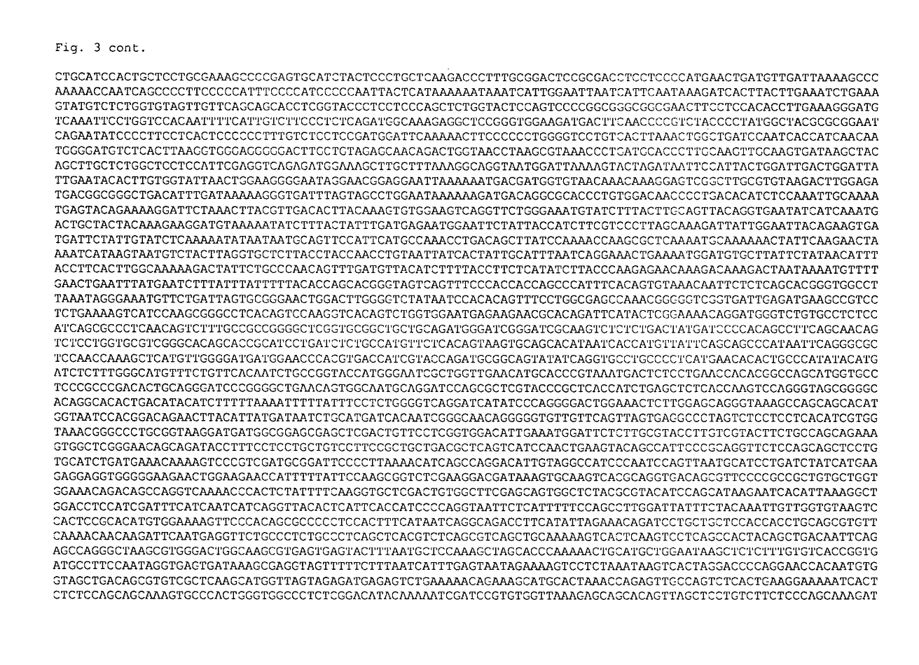

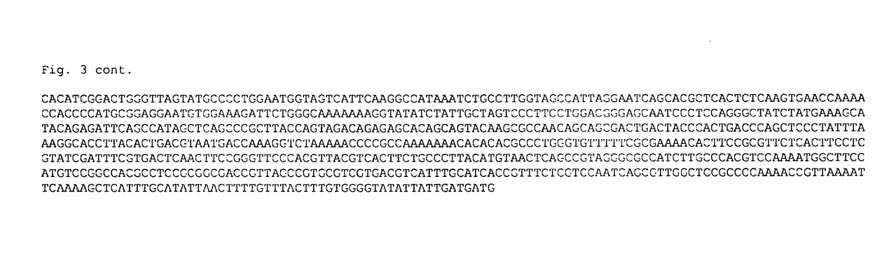

The total genome sequence of human adenovirus serotype 48 (Ad48) was determined using shot-gun sequencing techniques, generally as described for Ad11 and Ad35 in WO 00/70071. Hereto, purified wild-type Ad48 virus was inactivated by addition of 0.1 volume of (10×) proteinase K buffer (0.1M Tris-HCL [pH 7.9], 0.05M EDTA, 5% SDS) and 0.2 volume of proteinase K solution (Qiagen) and then heat inactivated at 56° C. for 3 h. Next, the viral DNA was isolated using the QIAamp MinElute Virus Spin Kit (QIAgen) according to the manufacturer's instructions. DNA was eluted from the spin column with 25 μl sterile TE. The obtained sequence of the human Ad48 genome (35206 nucleotides) is given in FIG. 2 (SEQ ID NO:2). Comparison with other adenovirus genomes or published fragments thereof reveals the same overall genome structure as known for all human adenoviruses. The overall homology between Ad48 (subgroup D) and Ad35 is 73.4%, which is significantly lower then the 98.1% homology seen between Ad35 and Ad11 viruses (which are both subgroup B viruses). The homology on the nucleotide level with another D-group adenovirus, human Ad9 (Genbank Accession No. AJ854486) is 91.6%. Table I presents the homology of the nucleic acid sequences encoding some of the predicted Ad48 proteins with their Ad9-, Ad5- and Ad35-derived counterparts. Clearly, the homology between the major capsid proteins (penton, hexon and fiber) that are important targets for neutralizing antibodies, of the three virus types from different subgroups (Ad5 and Ad35 versus Ad48), is low whereas the homology between Ad48 and Ad9 is much higher. The E3 region in Ad48, with coding regions located between nucleotide 25497 and 30747, differs in a number of aspects from the Ad5 and Ad35 E3 regions and has a structure similar to that of the subgroup D virus Ad9 (see for details e.g. Windheim and Burgert. 2002. J Virol. 76:755-766).

TABLE I
Percentages of homology of the proteins encoded
by viruses from Subgroup C (Ad5), B (Ad35) and D (Ad9)
with the corresponding predicted proteins of Ad48.

| | fiber | penton | hexon | E1B-55K | E4-orf6 |
|---|---|---|---|---|---|
| Ad5 | 35.9 | 70.7 | 77.0 | 50.9 | 63.1 |
| Ad35 | 33.1 | 74.8 | 80.6 | 54.5 | 69.2 |
| Ad9 | 65.0 | 91.2 | 90.1 | 99.4 | 98.6 |

### Example 2

### Generation of Recombinant Replication-Deficient Ad48 Viruses

Here, the construction of an Ad48 plasmid-based system to generate recombinant Ad48 vectors in a safe and efficient manner is described. The plasmid system consists of a first plasmid, referred to as an adapter plasmid, which contains Ad48 sequences 1 to 462 including the left ITR and packaging signal, an expression cassette and an Ad48 fragment corresponding to nucleotides 3362 to 5910. The expression cassette comprises the human CMV promoter, a multiple cloning site (MCS) and the SV40 polyadenylation signal (polyA) as previously described for Ad35 and Ad11 vectors (WO 00/70071). The adapter plasmid is based on pAdApt, albeit now generated to comprise the Ad48-derived sequences instead of the Ad5-derived sequences. Furthermore, the system consists of one or two other plasmids together constituting Ad48 sequences between nucleotide 3659 and 35206 (see FIG. 1 for the two- or three plasmid system that can be applied) that may be deleted for E3 sequences between nucleotide 26655 to 30736. In addition, the E4Orf6 (and therewith E4Orf6/7) sequences between 32241 and 33291 are preferably replaced by the corresponding E4 sequences from Ad5. This latter modification ensures efficient replication on Ad5-E1 complementing cell lines, like PER.C6® cells and 293 cells. The replacement of the E4Orf6 region of the backbone vector by the E4Orf6 region of Ad5 (being compatible with the E1B-55K protein produced by the packaging cell) has been described in WO 03/104467, WO 2004/001032, and U.S. Ser. No. 10/512,589, which applications are incorporated herein by reference, in their entirety.

Generation of Adapter Plasmid pAdApt48.Empty

First, plasmids that were used for harboring the Ad48 sequences were prepared. pAdApt containing the CMV, MCS and polyA termination signal was digested with PacI and AvrII, resulting in the generation of two digestion fragments of 5618 bp and 503 bp. The vector backbone (5618 bp) that was used in further cloning steps was isolated from gel using the Zymoclean DNA Clean & Concentrator-5 Kit (Zymo research). The digested vector was named pAdAptPac/Avr.

pAdApt35Bsu.LacZ was digested with PacI yielding two fragments of 2106 bp and 6137 bp. The plasmid backbone (2106 bp) was purified from gel using the gel extraction kit from Qiagen and subsequent dephosphorylated with SAP enzyme (Roche). The PacI pre-digested and SAP treated vector was designated pBr-PacI.

Then, two Ad48-specific PCR products were generated as follows: Fragment 1 (494 bp) was generated corresponding to Ad48 sequences 1-462 using primers Ad48(1-462) forw: 5′-CAG AAT TTA ATT AAT CGA CAT CAT CAA TAA TAT ACC CCA C-3′ (SEQ. ID. NO:3) and Ad48 (1-462) rev: 5′-CAG AAT CGC CTA GGT CAG CTG ATC TGT GAC ATA AAC-3′ (SEQ ID NO:4). This PCR introduces a PacI site at the 5′ end and an AvrII site at the 3′ end; another AvrII site was internally present in this fragment and located at nt position 334. PCR-reactions contained 1 μl viral DNA isolated as described above, 0.4 μM of each primer, 0.08 mM dNTP, 1× Phusion polymerase buffer (Finnzymes), 1 U Phusion (Finnzymes) and 3% DMSO. The program was set as follows: 30 sec at 98° C. followed by 30 cycles of 10 sec at 98° C., 30 sec at 58° C. and 2 min at 72° C., and ended by 8 min at 72° C. Fragment 1 was purified over agarose gel and digested with PacI. Subsequently, the completely digested fragment was partially digested (12.4 μg DNA, 12 min, either with 0.5 U or 0.8 U at 37° C.) with AvrII and purified over agarose gel. The PacI-, and partially AvrII-digested fragment was ligated to pAdAptPac/Avr. Following transformation into competent bacterial cells, DNA of recombinant clones was analyzed by HincII/NruI double digests. One selected clone containing the correct insert was designated pAdApt48 Fragment 1 and digested using BamHI and SalI. Digestion resulted in the generation of two restriction fragments of 2589 bp and 3499 bp. The fragment (3499 bp) representing the vector backbone (including fragment 1) was isolated from gel and was named pAdApt48 Fragment 1 (Bam/Sal).

Fragment 2 (2547 bp) was generated corresponding to Ad48 nucleotides 3362 to 5910 using primers Ad48 (3362-5909) forw: 5′-CAG AAT CGG GAT CCA GGT AGG TTT TGA GTA GTG GG-3′(SEQ ID NO:5) and Ad48(3362-5909) rev: 5′-CAG AAT ACG CGT CGA CTT AAT TAA TCT CGA GAG GGA ATA CCT AC-3′ (SEQ ID NO:6). This PCR introduces a BamHI site at the 5′ end and a SalI and PacI site at the 3′ end of the amplified fragment. Composition of the PCR reaction mixture, the program set up, and purification of the PCR product as well as the purification of the digested amplimers were performed as described above for fragment 1. Purified fragment 2 was (double) digested with BamHI and SalI, purified and ligated to pAdApt48 Fragment 1 (Bam/Sal). Following transformation into competent cells, DNA of recombinant clones was analyzed by various restriction enzyme digests. One selected clone containing the correct insert was designated pAdApt48.pac.sal.

This pAdApt48.pac.sal vector was then digested with PacI, generating two restriction fragments of 4050 bp and 2012 bp. The 4050 bp fragment was purified as described and ligated to pBr-PacI. Following electroporation into competent cells, DNA of recombinant clones was analyzed by PstI, PI-pspI, PacI and ApaLI digestions. One selected clone containing the correct insert was designated pAdApt48.empty. This adapter plasmid contains left-end Ad48 sequences (1-462 and 3362-5909) with the E1 region replaced by an expression cassette including the CMV promoter. The E1 deletion encompasses nucleotides 463-3361 comprising the full E1A and E1B coding regions.

### Generation of pBr.Ad48.SfiI-FseI

To enable cloning of an Ad48 SfiI restriction fragment (from Ad48-wt nt position 3751 to 17472) first a new plasmid was generated by inserting two PCR fragments in a pBr backbone. For this, two PCR fragments (fragment 3.1 and 3.2) were generated such that they could be ligated together (triple ligation) by using the (internally present) SbfI restriction site and cloned into a pBr-based backbone using the PacI restriction site.

In more detail, Fragment 3.1 (2283 bp) was generated containing Ad48 nucleotides 3659 to 5910 using primers SfiI-forw: 5′-CAG AAT TTA ATT AAC ATG ACA GCG ACG AGA CTG-3′ (SEQ ID NO:7) and Ad48 (3362-5909) rev: 5′-ACG CGT CGA CTT AAT TAA TCT CGA GAG GGA ATA CCT AC-3′ (SEQ ID NO:8). This PCR introduces a PacI site at the 5′ end and a SalI and PacI site at the 3′ end of the amplified fragment. Internal SfiI and SbfI sites are present at Ad48 nucleotide positions 3759 and 5593 respectively. Fragment 3.2 (3473 bp) was generated containing Ad48 nucleotides 17337 to 20796 using primers SbfI-forw: 5′-TGG AGA TGG AAG ATG CAA CTC-3′(SEQ ID NO:9) and FseI-Rpac: 5′-CAG AAT TTA ATT AAC AGC CGA AGG CGA GCC AG-3′ (SEQ ID NO:10). This PCR introduces a PacI site at the 3′ end of the amplified fragment. Internal SbfI, SfiI and FseI sites are present at Ad48-wt nucleotide positions 17415, 17450 and 20757 respectively.

The composition of the PCR-reaction mixtures for fragments 3.1 and 3.2 was similar to the one previously described for the generation of fragment 1. The program was set as follows: 30 sec at 98° C. followed by 30 cycles of 10 sec at 98° C., 30 sec at 58° C. and 2.5 min at 72° C., and ended by 8 min at 72° C. Fragments 3.1 and 3.2 were purified over gel and double digested with PacI and SbfI. The purified fragments were ligated in a triple ligation to pBR.PacI. Ligation mixture was incubated at room temperature for 2 h and 2 μl of the reaction was then electroporated into competent bacteria. DNA of recombinant clones was analyzed by ApaLI and NcoI digests. After plating, clones were analyzed for presence of the correct insert. This resulted in shuttle plasmid pBr.3.1/3.2.

In parallel, Ad48 wild type DNA was digested with SfiI and subsequently with AvrII to facilitate separation of the relevant fragment from undesired Ad48 sequences, followed by purification of the 13.7 kb SfiI fragment over gel. The thus isolated SfiI fragment was ligated to the SfiI digested and dephosphorylated vector pBr.3.1/3.2. Ligation mixtures were incubated and electroporated into competent bacteria. After plating, clones were checked by ScaI and AatII digestions. This resulted in plasmid pBr.Ad48.SfiI-FseI.

### Generation of pBr.Ad48.SbfI-rITR

pBr.Ad48.SbfI-rITR contains Ad48 sequences from the SbfI site at nucleotide 17415 to the end of the right inverted terminal repeat (rITR). To enable cloning of this sequence first a new plasmid was generated by inserting two PCR fragments in a pBr backbone. Hereto, two PCR fragments (fragment 4.1 and 4.2) were generated such that they could be ligated together (triple ligation) by using the (internally present) FseI restriction site and cloned into a pBr-based backbone using the PacI restriction site.

In more detail: Fragment 4.1 (1508 bp) was generated containing Ad48 nucleotides 33725 to the rITR (at position 35206) using primers: MluI-FseI: 5′-CAG AAT GGC CGG CCT CTA CGC GTA CAT CCA G-3′ (SEQ ID NO:11) and rITR-R: 5′-CAG AAT TTA ATT AAC ATC ATC AAT AAT ATA CCC CAC-3′ (SEQ ID NO:12). This PCR introduces an FseI site at the 5′ end and a PacI site at the 3′ end of the amplified fragment. An internal MluI site is present at Ad48-wt nucleotide positions 33729. Fragment 4.2 (3473 bp) was generated containing Ad48 nucleotides 17337 to 20796 using SbfI-Fpac: 5′-CAG AAT TTA ATT AAT GGA GAT GGA AGA TGC AAC TC-3′ (SEQ ID NO:13) and FseI-R: 5′-CAG CCG AAG GCG AGC CAG-3′ (SEQ ID NO:14). This PCR introduces a PacI site at the 5′ end and an FseI site is internally present in the fragment at Ad48-wt nucleotide position 20757.

The composition of the PCR reaction mixture, the program set up, and the purification of the PCR product and digested amplimers are the same as described for fragment 3.1 and 3.2 above. Purified fragments 4.1 and 4.2 were (double) digested with FseI and PacI, purified and ligated in a triple ligation to pBR.PacI that was also used for cloning fragments 3.1 and 3.2. Ligation mixture was incubated and electroporated into competent bacteria. Clones were analyzed by PacI/MluI double digestions for presence of the correct insert (expected fragments: 3.34, 2.11 and 1.48 kb). This resulted in shuttle plasmid pBr.4.1/4.2.

In parallel, Ad48-wt DNA was (double) digested with FseI and MluI, yielding restriction fragments of approximately 21, 13 and 1.5 kb, followed by purification of the desired 13 kb fragment over agarose. The isolated FseI/MluI fragment was ligated to the FseI/MluI pre-digested and dephosphorylated vector pBr.4.1/4.2. Ligation mixtures were electroporated into competent bacteria. Clones were analyzed by MluI/AvrII digestions for presence of the correct fragments (9, 6, 4 and 1 kb). This resulted in pBr.Ad48.SbfI-rITR.

### Generation of pBr.Ad48.SbfI-rITR.dE3

pBr.Ad48.SbfI-rITR was modified to delete part of the E3 region (nt: 26655-30736) to enlarge the cloning capacity. Hereto, two PCR fragments (fragments dE3-1 and dE3-2) were generated such that they could be ligated together in a triple ligation using the introduced (during PCR amplification) SpeI restriction site. Internally present AscI and SnaBI restriction sites were used to replace the corresponding fragment of fragment 4. Hereto, pBr.Ad48.SbfI-rITR was digested with SnaBI and AscI yielding two fragments: 13.5 kB and 6.5 kB. The fragment containing the vector backbone (13.5 kB) was purified over agarose and dephosphorylated. This vector was designated pBr.Ad48.SbfI-rITR (AscI/SnaBI).

In parallel, fragment dE3-1 (1224 bp) containing Ad48 nucleotides 25443 to 26655 was generated using primers: dE3AscI-F1: 5′-AAA GAC TAA GGC GCG CCC AC-3′ (SEQ ID NO:15) and Ad48dE3SpeIR1: 5′-CAG AAT ACT AGT GCA GGT GTT GGC TAC TGC TAG-3′ (SEQ ID NO:16). This PCR introduces a SpeI site at the 3′ end while an AscI site is present at the 5′ end. Fragment dE3-2 (1258 bp) containing Ad48 nucleotides 30736-31982 was generated using primers: Ad48dE3SpeIF2: 5′-CAG AAT ACT AGT CCA TGA ACT GAT GTT GAT TAA AAC-3′ (SEQ ID NO:17) and Ad48dE3SnaBI-R: 5′-TCC GCC AAG GTA GAC GTT AC-3′ (SEQ ID NO:18). This PCR introduces a SpeI site at the 5′ end and an SnaBI site at the 3′ end. The composition of the PCR reaction mixture, the program set up, and the purification of the PCR product and digested amplimers are the same as described for fragment 3.1 and 3.2 above.

Fragments dE3-1 and dE3-2 were pooled and digested with SpeI and purified. The eluted digested DNA fragments were ligated together. This ligation mixture was purified and then digested with AscI and SnaBI. Subsequently, digested DNA was purified and ligated to pBr.Ad48.SbfI-rITR (AscI/SnaBI) hereby replacing the original E3 sequence for similar sequence containing the E3 deletion (of 4081 bp). The resulting vector was named pBr.Ad48.SbfI-rITR.dE3.

Generation of pBr.Ad48.SbfI-rITR.dE3.5orf6

To allow efficient generation of recombinant Ad48 vectors on Ad5/E1-transformed cell lines, construct pBrAd48.SbfI-rITR.dE3 was further modified to contain E4orf6 and partial E4orf6/7 sequences from Ad5 replacing the corresponding sequences in Ad48. This strategy is fully in line with what has been explained in great detail in international application PCT/EP03/50125 (WO 03/104467), describing the generation of non-subgroup C adenoviruses on cell lines, such as the PER.C6 cells that express the E1 domain of an adenovirus of subgroup C. Hereto, three PCR fragments (5orf6-1, 5orf6-2 and 5orf6-3) were first generated and then assembled.

Fragment 5orf6-1 (349 bp) containing Ad48 nucleotides 31907 to 32243 was generated using primers Ad48.SnaBI-F: 5′-TCC TAC TAA TCC TAC AAC TCC-3′ (SEQ ID NO:19), and Ad48E4orf7-R: 5′-GGG AGA AAG GAC TGT GTA CAC TGT GAA ATG G-3′ (SEQ ID NO:20). Fragment 5orf6-2 (1128 bp) containing wt-Ad5 nucleotides 32962 to 34077 was generated using primers Ad48/Ad5E4orf6-F: 5′-CAC AGT GTA CAC AGT CCT TTC TCC CCG GCT-3′ (SEQ ID NO:21) and Ad48/Ad5E4orf6-R: 5′-AGA ATC CAC TAC AAT GAC TAC GTC CGG CG-3′ (SEQ ID NO:22). Fragment 5orf6-3 (461 bp) containing Ad48 nucleotides 33290 to 33739 was generated using primers Ad48E4orf4-F: 5′-GGA CGT AGT CAT TGT AGT GGA TTC TCT TGC-3′ (SEQ ID NO:23) and Ad26MluI-R: 5′-GAT GTA CGC GTA GAG CCA CT-3′ (SEQ ID NO:24). For the amplification of fragments 5orf6-1 and 5orf6-3, wt-Ad48 was used as template. For fragment 5orf6-2, plasmid pWE.Ad.AflII.rITR.dE3 (described in WO 99/55132) was used as template. The composition of the PCR reaction mixture, the program set up, and the purification of the PCR product and digested amplimers are the same as described for fragment 3.1 and 3.2 above. Purified fragments were mixed in approximate equimolar amounts and, in the presence of the outer border primers Ad48.SnaBI-F and Ad26MluI-R, subjected to an assembly PCR using Phusion DNA polymerase as described above. The program was set at 98° C. for 30 sec, followed by 5 cycles of 98° C. for 10 sec, 58° C. for 30 sec, and 2 min at 72° C. and ended with additional incubations at 72° C. for 2 min, followed by 98° C. for 30 sec, and continued by 30 cycles of 98° C. for 10 sec, 58° C. for 30 sec, and 2.5 min at 72° C. and ended with 8 min at 72° C. This resulted in a fused PCR product since fragment 2 is at the 5′ and 3′ end flanked by sequences that have overlap with fragment 1 and 3 respectively. The amplified fragment was purified over gel and digested by SnaBI and MluI, followed by purification of the 1837 bp fragment. Plasmid pBr.Ad48.SbfI-rITR.dE3 was also digested with SnaBI and MluI and the vector-containing fragment was purified over gel followed by dephosphorylation. The assembled and digested PCR fragment was ligated with the digested pBr.Ad48.SbfI-rITR.dE3. The ligation mixture was electroporated into competent bacteria. Clones were analyzed by SnaBI/MluI digestions for presence of the correct insert as determined by the expected restriction fragments of 14139 and 1835 bp. This resulted in plasmid pBr.Ad48.SbfI-rITR.dE3.5orf6.

### Generation of pWE.Ad48SfiI-rITR

The two described Ad48 fragments in plasmids pBr.Ad48.SfiI-FseI and pBr.Ad48.SbfI-rITR.dE3.5orf6 were then combined in a cosmid-based vector to make the generation of recombinant viruses even more efficient: it would require only one homologous recombination event, instead of two, to reconstitute a full recombinant genome with two ITRs and all genes necessary for replication in a Ad5-E1 transformed cell line (see FIG. 1). Hereto, construct pBr.Ad48.SfiI-FseI was digested with FseI and PacI and the 17.1 kb FseI-PacI fragment was isolated from gel. Furthermore, construct pBr.Ad48.SbfI-rITR.dE3.5orf6 was also digested with PacI and SbfI, and the 10.44 kb PacI/SbfI fragment was isolated from agarose. Lastly, construct pWE.Ad5.AflII-rITR.dE3 (described in WO 99/55132) was digested with PacI and the cosmid vector was isolated over gel. The purified cosmid vector was dephosphorylated. The isolated pWE vector was then ligated with the 17.1 kb SbfI-PacI fragment and the 10.4 kb PacI/SbfI fragment. Due to the large size, the resulting recombinant DNA was amplified using a phage packaging/bacteria infection protocol (Stratagene): Part of the ligation mixture was added to fresh packaging extracts (Stratagene) and incubated for 2 h at room temperature. After the packaging reaction was stopped, part of the mixtures containing the recombinant cosmid DNA was added to enable infection of freshly cultured STLB2 bacteria (Invitrogen) for 1 h at 37° C. in LB medium and were then plated and allowed to grow overnight at 37° C. Clones were analyzed by restriction analysis using various (combinations of) restriction enzymes for presence of the correct insert as determined by the expected restriction fragments. This resulted in a cosmid named pWE.Ad48SfiI-rITR.

Viruses were produced by introducing the adapter plasmid and the cosmid plasmid into PER.C6 cells by transfection systems known to the person skilled in the art. After initial generation of virus particles, these were further grown to significant titers in newly inoculated packaging cells, as described in detail elsewhere (WO 00/70071).

### Example 3

### Sequence of Human Adenovirus Serotype 26

The total genome sequence of human adenovirus serotype 26 (Ad26) was determined using shot-gun sequencing techniques as in Example 1 for Ad48. Hereto, Ad26 DNA was first isolated from purified virus particles. To 100 μl of virus stock, 12 μl 10× DNAse buffer (130 mM Tris-HCl pH 7.5; 1.2 mM CaCl₂; 50 mM MgCl₂) was added. After addition of 8 μl 10 mg/ml DNAse I (Roche Diagnostics), the mixture was incubated for 1 h at 37° C. Following addition of 2.5 μl 0.5 M EDTA, 3 μl 20% SDS and 1.5 μl Proteinase K, samples were incubated at 50° C. for 1 h. Next, the viral DNA was isolated using the Geneclean spin kit. DNA was eluted from the spin column with 25 μl sterile TE.

The obtained sequence (35155 nucleotides) is given in SEQ ID NO:1. Comparison with other adenovirus genomes or published fragments thereof reveals the same overall genome structure as known for all human adenoviruses. The overall homology between Ad26 (subgroup D) and Ad5 (subgroup C), and Ad35 (subgroup B) is 77.5% and 73.5% respectively, which is much lower then the 98.1% homology found between Ad35 and Ad11 viruses (both subgroup B). The homology on the nucleotide level with human Ad9 (Genbank Accession No. AJ854486), which is also a D-group virus, is 91.9%. Table II presents the homology of some of the predicted Ad26 proteins with their Ad9-, Ad5- and Ad35-derived counterparts. Clearly, the homology between the major capsid proteins (penton, hexon and fiber) that are important targets for neutralizing antibodies, of the three virus types from different subgroups (Ad5 and Ad35 versus Ad26), is low whereas the homology between Ad26 and Ad9 is much higher. The E3 region in Ad26, with coding regions located between nucleotide 25895 and 30762, differs in a number of aspects from the Ad5 and Ad35 E3 regions and has the structure as described for subgroup D viruses.

TABLE II
Percentages of homology of the proteins encoded
by viruses from Subgroup C (Ad5), B (Ad35) and D (Ad9)
with the corresponding predicted proteins of Ad26.

| | fiber | penton | hexon | E1B-55K | E4-orf6 |
|---|---|---|---|---|---|
| Ad5 | 33 | 72 | 76.7 | 51.1 | 63.1 |
| Ad35 | 26.2 | 74.7 | 79.9 | 54.7 | 68.9 |
| Ad9 | 61 | 99.8 | 90.1 | 100 | 97.6 |

It should be noted that in the course of sequencing different samples of Ad26 DNA, it became apparent that differences in sequence may exist between different isolates. The sequence that was finally used in the plasmids and cosmids that were subsequently applied to generate recombinant adenoviruses used in the present studies is given in SEQ ID NO:1. The discrepancies between this sequence and the several other isolates are provided in Table III. Here it is also indicated in which part of the genome the alteration occurs and whether it influences the resulting encoded protein (silent or coding mutation). The effects seen in amino acids of the encoded protein are indicated by the singular aa letter code. As some of these alterations occur in the coding regions of capsid proteins, they may have some influence on the immunogenicity.

TABLE III

| ORF | position | alteration | a.a. effect |
|---|---|---|---|
| VA-RNA | 6736 | T-C | Silent |
| 55K | 7780 | G-T | Silent |
| pIIIa | 8495 | G-A | Silent |
| pVII | 11493 | A-T | Silent |
| pVII | 11580 | T-C | Silent |
| pVII | 11583 | C-T | Silent |
| Hexon | 15597 | C-A | Silent |
| Hexon | 15600 | G-C | Silent |
| Hexon | 16686 | C-A | Silent |
| DBP | 18800-18803 | missing codon-TCT | E missing |
| 100K | 19583 | T-C | Silent |
| 100K | 19780 | T-C | Silent |
| 100K | 19950 | G-A | R → H |
| 100K | 19981 | A-G | Silent |
| 100K | 20005 | C-T | Silent |
| 100K | 20011 | T-C | Silent |
| 100K | 20017 | C-G | Silent |
| 100K | 20035 | T-C | Silent |
| 100K | 20185 | C-G | Silent |
| 100K | 20254 | C-A | Silent |
| 100K | 20260 | C-T | Silent |
| 100K | 20278 | G-A | Silent |
| 100K | 20296 | G-A | Silent |
| 100K | 20314 | G-T | D → E |
| 100K | 20320 | A-G | Silent |
| 100K | 20350 | C-T | Silent |
| 100K | 20413 | C-G | Silent |
| 100K | 20431 | A-G | Silent |
| 100K | 20503 | T-C | Silent |
| 100K | 20512 | A-T | Silent |
| 100K | 20515 | C-G | Silent |
| 100K | 20518 | C-G | I → M |
| 100K | 20539 | C-T | Silent |
| 100K | 20557 | A-C | Silent |
| 100K | 20689 | T-C | Silent |
| 100K | 20713 | T-C | Silent |
| 100K | 20740 | C-A | Silent |
| 100K | 20743 | G-A | Silent |
| 100K | 20770 | C-T | Silent |
| 100K | 20860 | A-G | Silent |
| 100K | 20875 | T-G | Silent |
| 100K | 20890 | T-C | Silent |
| 100K | 21043 | G-A | Silent |
| 100K | 21262-21263 | missing codon-AGC | S missing |
| 100K | 21271 | T-C | Silent |
| 100K | 21295 | A-G | Silent |
| 100K | 21434 | G-A | Silent |
| 100K | 21495 | C-A | Silent |
| pVIII | 21522-21524 | Extra: AGG | non coding |

### Example 4

### Generation of Recombinant Replication-Deficient Ad26 Viruses

This example describes the construction of an Ad26 plasmid-based system to generate recombinant Ad26 vectors in a safe and efficient manner. The plasmid system consists of a first plasmid, called the adapter plasmid, containing Ad26 sequences 1 to 471 including the left ITR and the Ad26 packaging signal, an expression cassette (for introducing a gene of interest) and an Ad26 fragment corresponding to nucleotides 3366 to 5913. The expression cassette comprises a human CMV promoter, a multiple cloning site (MCS) and the SV40 polyA as described for Ad35 and Ad11 vectors, and for Ad48 above. Furthermore, the system consists of one or two other plasmids together constituting Ad26 sequences between nucleotide 3763 and 35155 that may be deleted for E3 sequences between nucleotide 26689 to 30682, as E3 is not required for production and replication in packaging cells. In addition, the E4Orf6 and E4Orf6/7 sequences between 32166 and 33248 are to be replaced by the corresponding E4 sequences from Ad5. This latter modification ensures efficient replication on Ad5-E1 complementing cell lines, like PER.C6® and 293 cells, as discussed above.

Generation of Adapter Plasmid pAdApt26

Adapter plasmid pAdApt containing the CMV, MCS and polyA termination signal was digested with PacI and AvrII, resulting in the generation of two digestion fragments of 5618 bp and 503 nt. The vector backbone (5618 bp) that was used in further cloning steps was isolated from gel using the Zymoclean DNA Clean & Concentrator-5 Kit (Zymo research) according to manufacturers instructions. The digested vector was named pAdAptPac/Avr.

pAdApt was also digested with PacI and SalI, resulting in the generation of two digestion fragments of 2008 bp and 4113 bp. The vector backbone (2008 bp) that was used in further cloning steps was isolated from gel as described above. The digested vector was named pAdAptPac/Sal.

pAdApt35.Bsu.LacZ was digested with PacI yielding two fragments of 2106 bp and 6137 bp. The vector pBr plasmid backbone (2106 bp) was purified from gel using the gel extraction kit from Qiagen) and subsequent dephosphorylated with SAP enzyme. The PacI pre-digested and SAP treated vector was designated pBr-PacI.

Two Ad26 specific PCR products were generated as follows:

Fragment 1 (492 bp) was generated corresponding to Ad26 sequences 1-471 using primers
Ad49(1-462)forw: 5′-CCT TAA TTA ATC GAC ATC ATC AAT AAT ATA CCC CAC-3′ (SEQ ID NO:25) and Ad49(1-462)rev: 5′-CGC CTA GGT CAG CTG ATC TGT GAC ATA AAC-3′ (SEQ ID NO:26). The PCR introduces a PacI site at the 5′ end and an AvrII site at the 3′ end. PCR-reactions contained 1 μl viral DNA isolated as described above, 0.4 μM of each primer, 0.1 mM dNTP, 1× Phusion polymerase buffer (Finnzymes), 1 U Phusion (Finnzymes) and 3% DMSO. The program was set as follows: 30 sec at 98° C. followed by 30 cycles of 10 sec at 98° C., 30 sec at 58° C. and 2 min and 30 sec at 72° C., and ended by 8 min at 72° C. Fragment 1 was purified from gel and ligated to TOPO PCR4.1. DNA of recombinant clones was analyzed by PacI/AvrII double digests. The selected clone containing the correct (479 bp) insert was designated TOPO.Ad26lITR.
Fragment 2 (2579 bp) was generated corresponding to Ad26 nucleotides 3365 to 5913 using primers Ad26(3365-5913)-F: 5′-CAG AAG GGA TCC AGG TAG GTT TGA GTA GTG GG-3′ (SEQ ID NO:27) and Ad26(3365-5913)-R: 5′-CAA CGC GTC GAC TTA ATT AAT CTT GAG AGG GAA TAC CTA C-3′ (SEQ ID NO:28). The PCR introduces a BamHI site at the 5′ end and a SalI and PacI site at the 3′ end of the amplified fragment. Composition of the PCR reaction mixture, the program set up and purification of the PCR product as well as the purification of the digested amplimers were performed as previously described for fragment 1. Fragment 2 was purified after agarose gel electrophoresis using the GeneClean Turbo kit (Q-biogene) and ligated to TOPO PCR4.1 vector. DNA of recombinant clones was analyzed by BamHI/SalI double digests. The selected clone containing the correct (2562 bp) insert was designated TOPO.Ad26 overlap.
pAdApt26.Pac/Sal

Plasmid TOPO.Ad26lITR was digested with AvrII and PacI and the insert fragment was isolated from gel and ligated into pAdAptPac/Avr. DNA of recombinant clones was analyzed by PacI/AvrII double digests. The selected clone containing the correct insert was named pAdApt26.lITR.

Plasmid TOPO.Ad26overlap was digested with BamHI and SalI and the insert fragment was isolated as described above. Lastly, pAdApt26.lITR was digested with BglII and SalI and the 3.5 kb vector-containing fragment was isolated from gel. Isolated DNA was dephosphorylated. The isolated vector fragment and the insert were ligated, resulting in pAdApt26.pac/sal.

Plasmid pAdApt26.pac/sal was digested with PacI and the 4 kb AdApt26 insert fragment was ligated to pBr-PacI, and resulted in pAdApt26: the adapter plasmid containing left end Ad26 sequences with the E1 region replaced by an expression cassette with the CMV promoter. The E1 deletion relates to nucleotide 472-3365 comprising the full E1A and E1B coding regions.

Generation of pBrAd26SfiI

To enable cloning of an Ad26 SfiI restriction fragment (from Ad26-wt nt position 3762 to 17466) first plasmid pBrAd49SfiI (patent application U.S. Ser. No. 11/140,418) was digested with SfiI followed by purification of the 2.1 kb vector fragment from gel. Isolated DNA was dephosphorylated.

In parallel, Ad26-wt DNA was digested with SfiI followed by purification of the 13.7 kb SfiI fragment from gel. The thus isolated SfiI fragment was ligated to the isolated and dephosphorylated 2.1 kb vector fragment derived from pBr.Ad49.SfiI. This resulted in plasmid pBr/Ad26.SfiI.

Generation of pBrAd26.SrfI-rITR

pBrAd26.SrfI-rITR contains Ad26 sequences from the SrfI site at nucleotide 15433 to the end of the right inverted terminal repeat (rITR). To enable cloning of this sequence first a new plasmid was generated by inserting two PCR fragments in a pBr backbone. Hereto, two PCR fragments (fragment 4.1 and 4.2) were generated such that they could be ligated together (triple ligation) by using the (internally present) SbfI restriction site and cloned into a pBr-based backbone using the PacI restriction site.

In more detail:
Fragment 4.1 (1517 bp) was generated corresponding to Ad26 nucleotides 33666 to 35155 (including rITR) using primers: MluI-F: 5′-CAG AAT CCT GCA GGC TCT ACG CGT ACA TCC AG-3′ (SEQ ID NO:29) and rITR-R: 5′-CAG AAT TTA ATT AAC ATC ATC AAT AAT ATA CCC CAC-3′ (SEQ ID NO:30). The PCR introduces an SbfI site at the 5′ end and a PacI site at the 3′ end of the amplified fragment. An internal MluI site is present at Ad26-wt nucleotide positions 33670.
Fragment 4.2 (2111 bp) was obtained using SrfI-F: 5′-CAG AAT TTA ATT AAA CTA TGC CAG ACG CAA GAG C-3′ (SEQ ID NO:31) and SbfI-R: 5′-CTC GTA CGA GGG CGG CTC-3′ (SEQ ID NO:32). This PCR introduces a PacI site at the 5′ end and a SbfI site is internally present in the fragment at Ad26-wt nucleotide position 17431. The PCR program was set as follows: 30 sec at 98° C. followed by 30 cycles of 10 sec at 98° C., 30 sec at 58° C. and 2 min at 72° C., and ended by 8 min at 72° C. Fragment 1 was purified after agarose gel electrophoresis using the GeneClean Turbo kit (Q-bio gene) and purified fragments 4.1 and 4.2 were pooled and digested with SbfI, purified and ligated. After purification, the fragment was digested with PacI, purified and ligated to pBR.PacI. This resulted in shuttle plasmid pBr/Ad26-4.1+4.2, which was subsequently digested with MluI. The linear fragment (5679 bp) was digested with SrfI. The Srf-Mlu restriction fragment (3674 bp) representing the vector was gel purified and named pBr/Ad26-4.1+4.2 (Srf-MluI).

In parallel, Ad26-wt DNA was digested first with SrfI and MluI, yielding restriction fragments of 18237, 15433 and 1485 bp, followed by purification of the 18237 by fragment from gel. This purified fragment was ligated with pBr/Ad26-4.1+4.2 (Srf-MluI). This resulted in plasmid pBr/Ad26.SrfI-rITR.

Generation of pBr/Ad26.SrfI-rITR^{˜}dE3

Plasmid pBr/Ad26.SrfI-rITR was modified to delete part of the E3 region (nt. 26683-30683) to enlarge the cloning capacity. Hereto, two PCR fragments (fragments E3-1 and E3-2) were generated and ligated using the introduced (during PCR amplification) SpeI restriction site. Internally present AscI (nt. 25487) and EcoRI (nt. 31725) sites in these fragments were used to replace the corresponding fragment of fragment 4 in pBr/Ad26.SrfI-rITR. pBr/Ad26.SrfI-rITR was digested with AscI and EcoRI generating two restriction fragments of 15627 and 6238 bp. The 15627 bp restriction fragment representing the vector backbone was isolated from gel and designated pBr/Ad26.SrfI-rITR (EcoRI-AscI).

In parallel, Fragment E3-1 (1224 bp) was generated corresponding to Ad26 nucleotides 25477 to 26690 using primers: dE3AscI-F1: 5′-AAA GAC TAA GGC GCG CCC AC-3′ (SEQ ID NO:33) and dE3SpeI-R1: 5′-CAG AAT ACT AGT GCA GTG AGT GTT GGA GAC TGC-3′ (SEQ ID NO:34). This PCR introduces a SpeI site at the 3′. An AscI site is present at the 5′ end (located at nt-position 25487).

Fragment E3-2 (1069 bp) was generated corresponding to Ad26 nucleotides 30683-31740 using primers set: dE3SpeI-F2: 5′-CAG AAT ACT AGT CCA TGA ACT GAT GTT GAT TAA AAG-3′ (SEQ ID NO:35) and Ad26dE3EcoRI-R2: 5′-GAT GGT AAT AGA ATT CCA TTC TC-3′ (SEQ ID NO:36). This PCR introduces a SpeI site at the 5′ end. An EcoRI site is present at the 3′ end (located at nt. 31725). Composition of the PCR reaction mixture as well as the PCR program set up was similar as previously described for fragment 4.1 and 4.2. Both PCR fragments were gel purified.

Purified fragments E3-1 and E3-2 were pooled and digested with SpeI, and ligated together. Subsequently (SpeI digested) DNA was digested with AscI and EcoRI and ligated to pBr/Ad26.SrfI-rITR (EcoRI-AscI). This resulted in pBr/Ad26.SrfI-rITR.dE3, in which the original E3 sequence was replaced by the similar sequence now containing a deletion in E3.

Generation of pBr/Ad26.SrfI-rITR.dE3.5Orf6

To allow efficient generation of recombinant Ad26 vectors on Ad5-transformed cell lines, construct pBrAd26.SbfI-rITR.dE3 was further modified to contain E4-Orf6 and partial E4Orf6/7 sequences from Ad5 replacing corresponding sequences in Ad26. Hereto, three PCR fragments (5orf-1, 5orf6-2 and 5orf6-3) were first generated and then assembled:

Fragment 5orf6-1 (477 bp) was generated using primers Ad26.EcorI-F: 5′-GAG AAT GGA ATT CTA TTA CCA TC-3′ (SEQ ID NO:37), and Ad49E4orf7-R: 5′-GGG AGA AAG GAC TGT TTA CAC TGT GAA ATG G-3′ (SEQ ID NO:38).
Fragment 5orf6-2 (1104 bp) was generated using primers Ad5E4orf6-F: 5′-CAC AGT GTA AAC AGT CCT TTC TCC CCG GCT-3′ (SEQ ID NO:39) and Ad26/Ad5E4orf6-R: 5′-AGA ATC CAT TTC AAT GAC TAC GTC CGG CG-3′ (SEQ ID NO:40).
Fragment 5orf6-3 (461 bp) was generated using primers Ad26E4orf4-F: 5′-GGA CGT AGT CAT TGA AAT GGA TTC TCT TGC-3′ (SEQ ID NO:41) and Ad26MluI-R: 5′-GAT GTA CGC GTA GAG CCA CT-3′ (SEQ ID NO:42).

For the amplification of fragments 5orf6-1 and 5orf6-3, wt-Ad26 was used as template and for fragment 5orf6-2, pWe.Ad.AflII.rITR.fib5.dE3 was used as template. Composition of the PCR reaction mixture, the program set up, purification of the PCR products were performed as previously described for fragment 3.1 and 3.2 except that in this current PCR 2 μl instead of 0.5 μl of template was used. Purified fragments were mixed in approximate equimolar amounts and, in the presence of the outer border primers Ad26.EcorI-F and Ad26MluI-R, subjected to an assembly PCR using a PCR reaction composition including Phusion DNA polymerize as described above. The program was set at 98° C. for 30 sec, followed by 5 cycles of 98° C. for 10 sec, 58° C. for 30 sec, and 2 min at 72° C. and ended with additional incubations at 72° C. for 2 min, and at 98° C. for 30 sec, and continued by 30 cycles of 98° C. for 10 sec, 58° C. for 30 sec, and 2.5 min at 72° C. and ended with 8 min at 72° C. This resulted in a fused PCR product since fragment 5orf6-2 is at the 5′ and 3′ end flanked by sequences that have overlap with fragment 5orf6-1 and 5orf6-3 respectively. The amplified fragment was purified from gel and digested with EcoRI and MluI followed by purification. Plasmid pBr/Ad26.SrfI-rITR.dE3 was also digested with EcoRI and MluI and the vector-containing fragment was purified from gel. The assembled and digested PCR fragment was ligated with EcoRI-MluI digested pBr/Ad26.SrfI-rITR.dE3, which resulted in plasmid pBr/Ad26.SrfI-rITR.dE3.5orf6.

Generation of pWE.Ad26.dE3.5orf6

The two described Ad26 fragments in plasmids pBr.Ad26.SfiI and pBr.Ad26.SrfI-rITR.dE3.5orf6 were combined in a cosmid-based vector to generate recombinant viruses even more efficiently, requiring only one homologous recombination instead of two to reconstitute a full recombinant genome with two ITR's and all genes necessary for replication in a Ad5-E1 transformed cell line. Hereto, construct pBr.Ad26.SfiI was digested with PacI and SrfI and the 11.69 kb restriction fragment was isolated from gel. Furthermore, construct pBr.Ad26.SrfI-rITR.dE3.5orf6 was digested with PacI and SrfI, and the 15.80 kb restriction fragment was isolated. Lastly, construct pWE.Ad5.AflII-rITR.dE3 (described in WO 99/55132) was digested with PacI and dephosphorylated. The linearized and SAP treated pWE vector (8.12 kb) was then ligated to the 11.69 and 15.80 kb PacI-SrfI fragments described above. This resulted in a cosmid named pWE.Ad26.dE3.5orf6.

Introduction of the transgenes into both the Ad26 and Ad48 recombinant adenovirus genomes was generally performed as described elsewhere (Lemckert A A et al. 2005. J Virol 79:9694-9701) and viruses were generated using PER.C6 cells, and purified subsequently using methods widely applied and as known to the person skilled in the art.

### Example 5

### Seroprevalence in South African Pediatric Cohorts

As pediatric populations may very likely form the larger part of the target group requiring vaccination against life-threatening diseases such as AIDS, the seroprevalence within such group was investigated in respect to Ad5, Ad26, Ad48 and Ad49. The cohort of 501 samples was (relatively arbitrarily) separated into four different age groups: 1-2 years, 3-6 years, 7-12 years and 12-18 years.

Neutralization was determined generally as described in Example 1 of WO 00/70071 and according to Sprangers M C et al. (2003; J Clin Microbiol 41: 5046-5052). The results of these neutralization studies are given in Table IV (Ad5), Table V (Ad26), Table VI (Ad48) and Table VII (Ad 49).

Titers of <16 are regarded as negative by this assay, 16-200 is low, 200-1000 is high, and >1000 is considered very high. The categories are fairly arbitrary, but it is suspected that titers >200 will likely be suppressive, according to data known in the art. The higher the titers, the lower the predicted efficacy. In the tables, the samples were divided by age groups. The numbers in the boxes represent the percentage of the samples in a particular age group with a particular titer range.

Clearly, if one assesses the neutralization present in such samples against Ad5, it becomes immediately clear that there is an increasing seroprevalence with increasing age: percentages of samples that have titers over 1000 increase from 2.5% in the 1-2 year group, to 47.7% in the 12-18 year group. Although this was not further studied in detail, it suggests that age groups of higher age will show a further increase in this percentage. It is concluded that at least in this (limited) cohort, most children over 2 years of age are Ad5 seropositive, which again indicates the disadvantage of using a vaccine comprising a recombinant adenovirus based solely on this serotype, as the neutralizing activity present in these individuals may hamper the efficacy of the vaccine. With respect to the three serotypes from subgroup D, Ad26, Ad48 and Ad49, it becomes immediately clear that neutralizing activity is present in significantly less samples in comparison to Ad5. While some samples, ranging from 1.7 to 2.1% in the age groups from 6-18 have titers above 1000, none of the samples in these age groups reached such titers against Ad48 and Ad49. Especially, it is worth noticing that the neutralization against Ad48 remains dramatically low over time: In the age group of 12-18, still over 90% of the samples were regarded as non-neutralizing (Table VI), which shows that a recombinant adenovirus based on Ad48 is most preferred when applying vaccines comprising such gene delivery vehicle is considered, at least when children are concerned.

TABLE IV
Ad5 seroprevalence

| % | <16 | 16-200 | 200-1000 | >1000 | TOTAL |
|---|---|---|---|---|---|
| 1-2 yrs | 71.79 | 15.38 | 10.26 | 2.56 | 100.00 |
| 2-6 yrs | 46.15 | 15.38 | 12.31 | 26.15 | 100.00 |
| 6-12 yrs | 26.81 | 14.49 | 18.12 | 40.58 | 100.00 |
| 12-18 yrs | 20.69 | 16.67 | 14.94 | 47.70 | 100.00 |

TABLE V
Ad26 seroprevalence

| % | <16 | 16-200 | 200-1000 | >1000 | TOTAL |
|---|---|---|---|---|---|
| 1-2 yrs | ND | ND | ND | ND | ND |
| 2-6 yrs | 77.50 | 10.00 | 7.50 | 5.00 | 100.00 |
| 6-12 yrs | 78.26 | 12.32 | 7.25 | 2.17 | 100.00 |
| 12-18 yrs | 77.59 | 15.52 | 5.17 | 1.72 | 100.00 |

TABLE VI
Ad48 seroprevalence

| % | <16 | 16-200 | 200-1000 | >1000 | TOTAL |
|---|---|---|---|---|---|
| 1-2 yrs | ND | ND | ND | ND | ND |
| 2-6 yrs | 90.00 | 10.00 | 0.00 | 0.00 | 100.00 |
| 6-12 yrs | 92.03 | 7.25 | 0.72 | 0.00 | 100.00 |
| 12-18 yrs | 90.23 | 8.62 | 1.15 | 0.00 | 100.00 |

TABLE VII
Ad49 seroprevalence

| % | <16 | 16-200 | 200-1000 | >1000 | TOTAL |
|---|---|---|---|---|---|
| 1-2 yrs | 98.66 | 1.34 | 0.00 | 0.00 | 100.00 |
| 2-6 yrs | 80.00 | 17.50 | 2.50 | 0.00 | 100.00 |
| 6-12 yrs | 81.88 | 18.12 | 0.00 | 0.00 | 100.00 |
| 12-18 yrs | 75.29 | 21.26 | 3.45 | 0.00 | 100.00 |

### Example 6

### In Vivo Immunogenicity of Recombinant Ad26, Ad35 and Ad48 Carrying a Nucleic Acid Encoding an Antigen (in Mice)

Next, it was studied whether recombinant replication-defective adenoviruses based on three low-neutralized serotypes, Ad26, Ad35 and Ad48 were able to elicit a significant immune response in vivo. For this, vectors were generated that all contained the SIVmac239 Gag insert from Simian Immunodeficiency Virus. Recombinant DNA such as the required adapter plasmids, and the recombinant viruses were generated generally as described (Lemckert A A et al. 2005. J Virol 79:9694-9701).

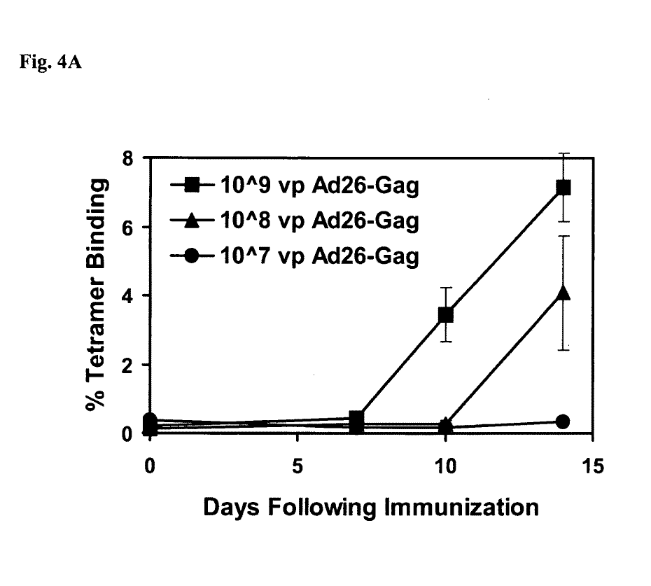

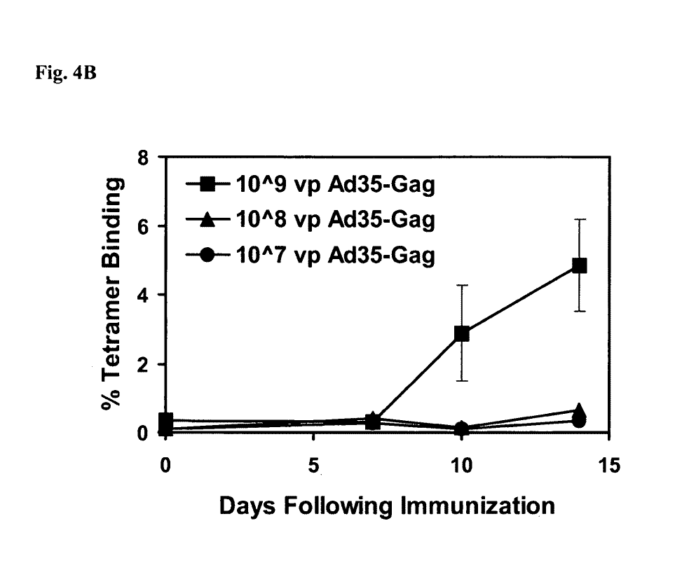

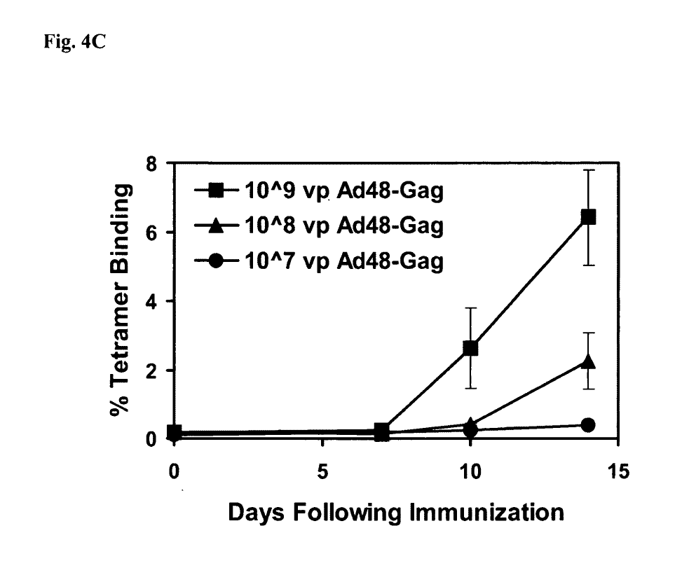

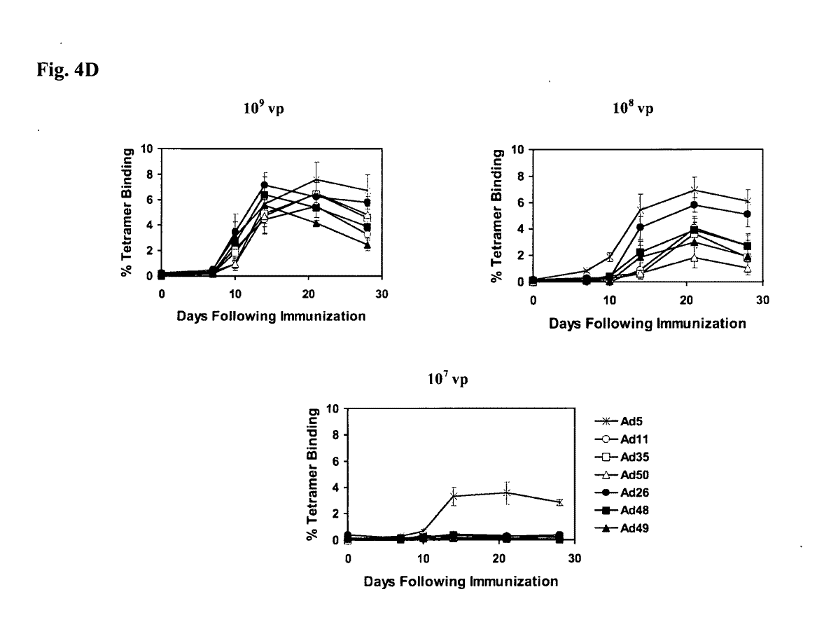

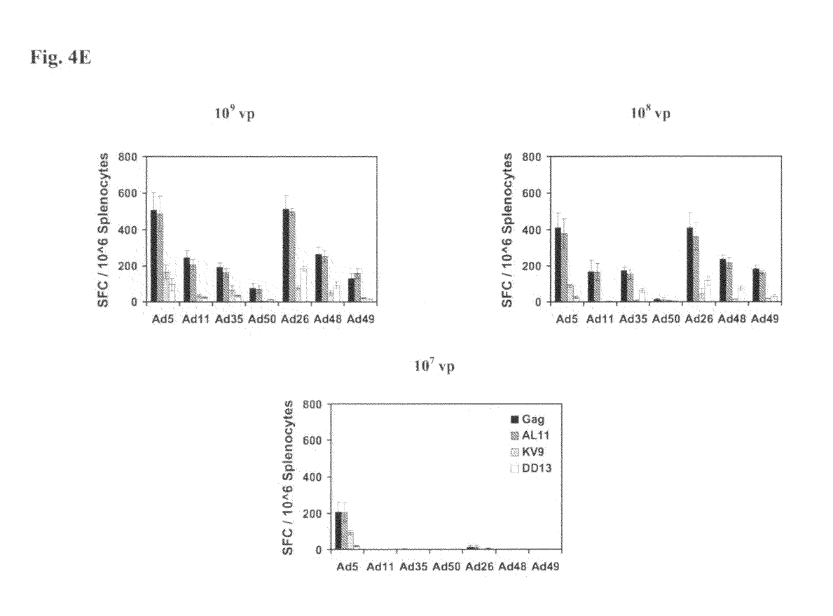

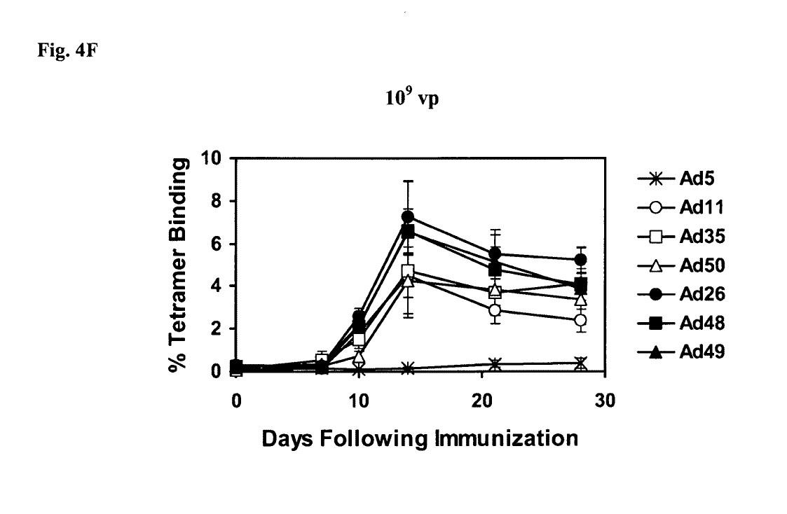

Eight C57/BL6 mice per group were injected intramuscularly with different amounts of viral vectors: 10⁷, 10⁸ and 10⁹ viral particles (vp). All vaccination procedures and cellular immune responses were performed and measured by assessing the CD8+ T cell response via D^{b}/AL11 tetramer binding assays as previously described (Barouch D H et al. 2004. J Immunol 172:6290-6297) and by interferon-γ (IFN-γ) ELISPOT assays as described (Barouch D H et al. 2004. J Immunol 172:6290-6297; Nanda A et al. 2005. J Virol 79:14161-8). Overlapping 15 amino acid peptides spanning the SIVmac239 Gag protein were obtained from the NIH AIDS Research and Reference Reagent Program. 96-well multiscreen plates (Millipore, Bedford, Mass.) were coated overnight with 100 μl/well of 10 μg/ml anti-mouse or anti-human IFN-γ (BD Pharmingen, San Diego, Calif.) in endotoxin-free Dulbecco's PBS (D-PBS). The plates were then washed three times with D-PBS containing 0.25% Tween-20 (D-PBS/Tween), blocked for 2 h with D-PBS containing 5% FBS at 37° C., washed three times with D-PBS/Tween, rinsed with RPMI 1640 containing 10% FBS to remove the Tween-20, and incubated with 2 μg/ml each peptide and 5×10⁵ murine splenocytes or 2×10⁵ rhesus monkey PBMC in triplicate in 100 μl reaction volumes. Following an 18 h incubation at 37° C., the plates were washed nine times with PBS/Tween and once with distilled water. The plates were then incubated with 2 μg/ml biotinylated anti-mouse or anti-human IFN-γ (BD Pharmingen, San Diego, Calif.) for 2 h at RT, washed six times with PBS/Tween, and incubated for 2 h with a 1:500 dilution of streptavidin-alkaline phosphatase (Southern to Biotechnology Associates, Birmingham, Ala.). Following five washes with PBS/Tween and one with PBS, the plates were developed with nitro blue tetrazolium/5-bromo-4-chloro-3-indolyl-phosphate chromogen (Pierce, Rockford, Ill.), stopped by washing with tap water, air dried, and read using an ELISPOT reader (Cellular Technology Ltd., Cleveland, Ohio). Spot-forming cells (SFC) per 10⁶ cells were calculated. Media backgrounds were consistently <15 SFC per 10⁶ cells. The results are shown in FIG. 4A (Ad26), 4B (Ad35) and 4C (Ad48).

From these results it can be concluded that the immunogenicity elicited by the subgroup B derived viral vector based on Ad35 is lower than the immune response found by using the subgroup D derived viral vectors based on Ad26 and Ad48. For instance, while a response with 10⁸ vp is not determined 15 days after injection with recombinant Ad35-SIVmac239 Gag virus, there is a significant response found with this amount of virus in the case of vaccines based on Ad26 and Ad48. As discussed herein, the differences between subgroup C or D and subgroup B viruses with respect to immune responses in vivo may very well be due to the difference in affinity towards different cellular receptors, while also their different tropism may contribute to such differences. In any case, whatever the reason for the observed immune responses, it is clear that Ad26 and Ad48 provide an even stronger response than Ad35 in vivo and therefore provide two very good alternatives with respect to the application of low-neutralized serotypes in therapy.

The in vivo immunogenicity responses to the recombinant Ad26, Ad35 and Ad49 vectors were further monitored for 30 days and plotted against the results obtained with recombinant Ad5 (subgroup C), Ad11 and Ad50 (subgroup C) and Ad49 (subgroup D), which were all constructed in the same manner as Ad26 and Ad48 and all carrying the same antigen. Responses were monitored for 30 days following immunization. The results of these experiments with the three different doses are shown in FIG. 4D (tetramer binding) and 4E (SFC/10⁶ splenocytes): Vaccine-elicited CD8⁺ T lymphocyte responses specific for the dominant AL11 epitope (AAVKNWMTQTL; SEQ ID NO:43) were monitored by D^{b}/AL11 tetramer binding assays at multiple time points following immunization. As shown in FIG. 4D, all rAd vectors were highly immunogenic at the high dose of 10⁹ vp. At the intermediate dose of 10⁸ vp, rAd5 and rAd26 vectors proved significantly more immunogenic than rAd11, rAd35, rAd50, rAd48, and rAd49 vectors (P<0.01 comparing responses on day 28 using ANOVA). At the low dose of 10⁷ vp, rAd5 vectors still elicited Gag-specific cellular immune responses, whereas none of the rare serotype rAd vectors elicited detectable responses. These data suggest a relative hierarchy of vector immunogenicity in mice. In C57BL/6 mice without anti-Ad5 immunity, rAd5-Gag was consistently the most immunogenic vector among those studied. Among the six rare serotype rAd vectors, rAd26-Gag appeared the most immunogenic. This hierarchy was confirmed by functional IFN-γ ELISPOT assays performed on day 35 using a Gag peptide pool, the CD8⁺ T lymphocyte epitopes AL11 (AAVKNWMTQTL; SEQ ID NO:43) and KV9 (KSLYNTVCV; SEQ ID NO:44), and the CD4⁺ T lymphocyte epitope DD13 (DRFYKSLRAEQTD; SEQ ID NO:45) shown in FIG. 4E.

Since the majority of individuals in the developing world have high levels of pre-existing anti-Ad5 immunity, this study was repeated in mice with anti-Ad5 immunity. C57BL/6 mice (n=4/group) were pre-immunized with two injections of 10¹⁰ vp rAd5-Empty separated by a four week interval. This regimen induced Ad5-specific NAb titers of 8,192-16,384 (data not shown), which models the upper 20% of Ad5-specific NAb titers found in individuals in sub-Saharan Africa (Barouch D H et al. 2004. J Immunol 172:6290; Lemckert et al. 2005. J Virol 79:9694-701). Four weeks later, these mice were primed with 10⁹ vp of each vector, and vaccine-elicited cellular immune responses were evaluated by D^{b}/AL11 tetramer binding assays and IFN-γ ELISPOT assays. As shown in FIGS. 4F and G, the immunogenicity of rAd5-Gag was essentially ablated by high levels of anti-Ad5 immunity. In contrast, the immunogenicity of the six rare serotype rAd-Gag vectors was not detectably suppressed in mice with anti-Ad5 immunity as compared with naïve mice (FIG. 4D-E). Importantly, all rare serotype rAd-Gag vectors proved significantly more immunogenic than rAd5-Gag in the presence of pre-existing anti-Ad5 immunity (P<0.001).

### Example 7

### In Vivo Immunogenicity of Recombinant Ad26, Ad35 and Ad48 Carrying a Nucleic Acid Encoding an Antigen (in Primates)

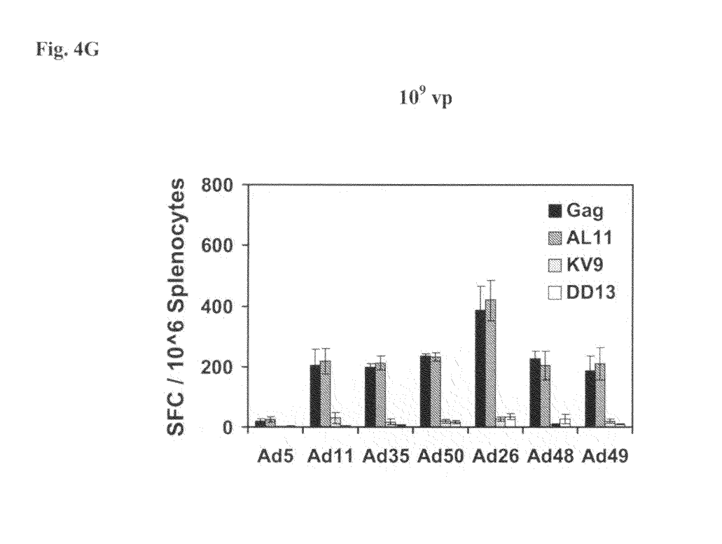

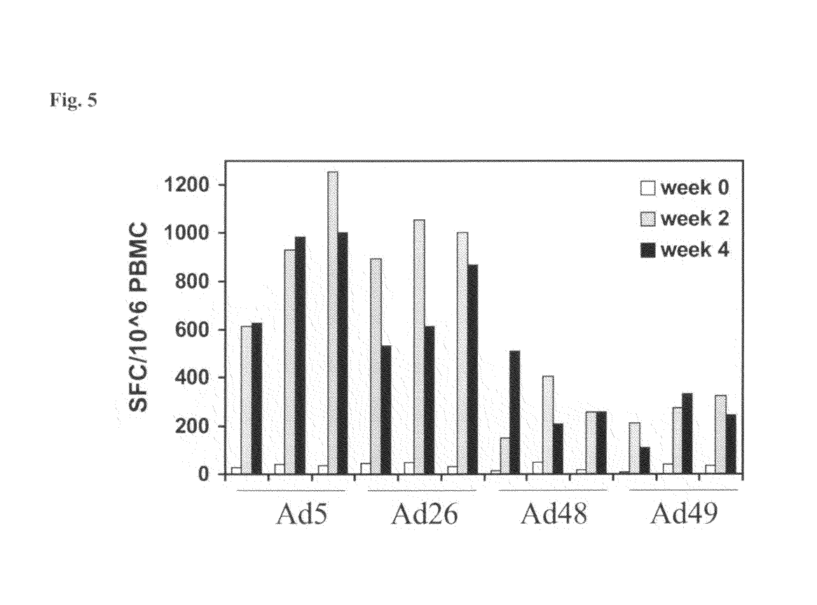

In line with the experiments described in Example 6, further in vivo studies were performed in primates. For this, in total 12 outbred rhesus monkeys without pre-existing immunity against Ad5 were injected with a single dose of 10¹¹ vp recombinant Ad5-Gag, Ad26-Gag, Ad48-Gag and Ad49-Gag (3 monkeys per group). These viruses were all carrying the SIVmac239 Gag insert from Simian Immunodeficiency Virus, as described above. At week 2 and at week 4 post infection, Gag-specific cellular immune responses were assessed by the IFN-γ ELISpot as discussed above. The results are shown in FIG. 5.

Clearly, as expected, the Ad5 vector is able to induce a proper immune response against the Gag insert, in the absence of pre-existing immunity against the Ad5 virus. The Ad48 and Ad49 showed a lower induction of the cellular immune response in comparison to the Ad5 vector. However, in these primates, recombinant Ad26-Gag yields an immune response against the antigen that is comparable to the response induced by the Ad5 vector. Clearly, this adds to the beneficial use of this recombinant D-type virus in therapeutic settings.

### Example 8

### Heterologous Prime-Boost Regimens in Mice

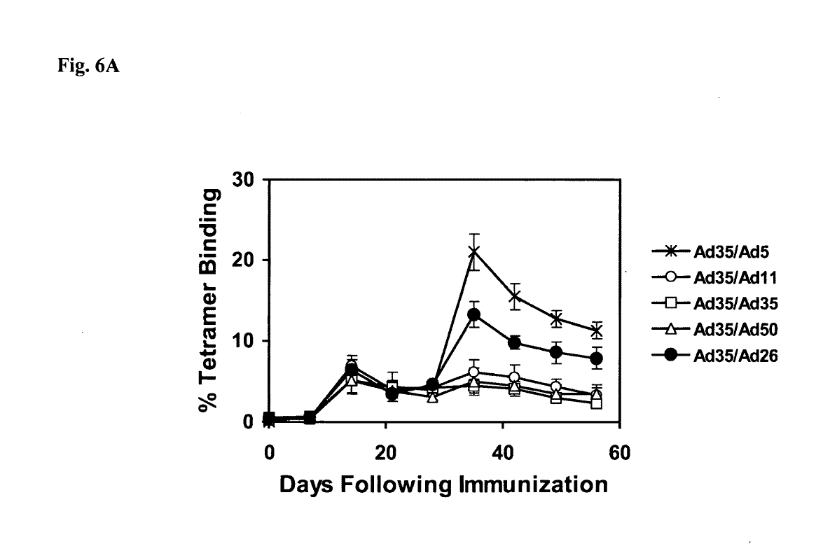

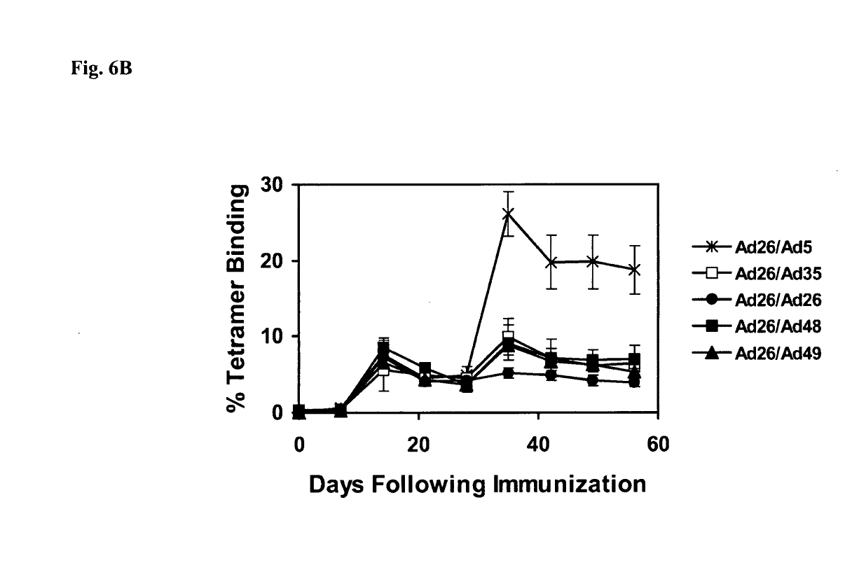

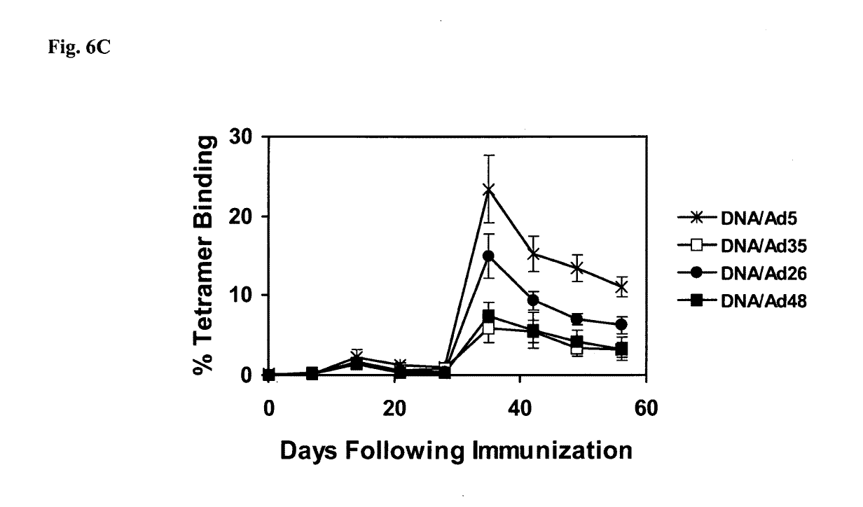

Heterologous prime-boost regimens consisting of two rAd vectors derived from different serotypes can be administered to avoid the generation of anti-vector immunity following immunization. As shown in FIG. 6A, naïve C57BL/6 mice (n=4/group) were primed i.m. on day 0 with 10⁹ vp rAd35-Gag and boosted on day 28 with 10⁹ vp rAd5-Gag, rAd11-Gag, rAd35-Gag, rAd50-Gag, or rAd26-Gag. Boosting with rAd5-Gag expanded mean tetramer⁺CD8⁺ T lymphocytes responses to 21.0% on day 35, which declined to 11.3% on day 56. Boosting with rAd26-Gag also expanded tetramer⁺CD8⁺ T lymphocytes responses, although to a lesser degree (13.3% on day 35; 7.8% on day 56). In contrast, boosting with rAd11-Gag, rAd35-Gag, and rAd50-Gag proved substantially less effective, consistent with recent findings that rAd11 and rAd35 vectors from subgroup B elicit cross-reactive vector-specific Nabs (Lemckert et al. 2005. J Virol 79:9694-701; Thorner et al. 2006. J Virol 80:12009-16). These data suggest that immune responses primed by rAd35 vectors from subgroup B are boosted more effectively by rAd vectors from subgroups C (exemplified by rAd5) or D (exemplified by rAd26) as compared with other rAd vectors from subgroup B. Clearly, because Ad26 does not encounter neutralizing activity in the majority of the population, a prime/boost set-up in which a subgroup B adenovirus prime such as with rAd35 is followed by a boost with a serogroup D vector, preferably rAd26 is preferred over an Ad35/Ad5 prime/boost.

As shown in FIG. 6B, additional groups of naïve C57BL/6 mice (n=4/group) were primed i.m. on day 0 with 10⁹ vp rAd26-Gag and boosted on day 28 with 10⁹ vp rAd5-Gag, rAd35-Gag, rAd26-Gag, rAd48-Gag, or rAd49-Gag. Boosting with rAd5-Gag expanded mean tetramer⁺CD8⁺ T lymphocytes responses to 26.1% on day 35, which declined to 18.7% on day 56. Boosting with rAd35-Gag, rAd48-Gag, and rAd49-Gag also expanded tetramer⁺CD8⁺ T lymphocytes responses, although to a lesser degree (8.8-9.9% on day 35; 5.3-7.0% on day 56). As expected, boosting with the homologous rAd26-Gag vector did not detectably enhance responses, likely due to neutralizing antibodies induced by the priming.

These data suggest that immune responses primed by rAd26 vectors from subgroup D are boosted effectively by rAd5 vectors from subgroup C, but they can also be boosted by heterologous rAd vectors from subgroup B as well as subgroup D.

Priming with plasmid DNA vaccines and boosting with rAd5 vectors has been shown in the art to elicit particularly potent antigen-specific immune responses. To explore the relative capacity of the rare serotype rAd vectors of the present invention to boost responses primed by DNA vaccines, C57BL/6 mice (n=4/group) were immunized i.m. on day 0 with 50 μg plasmid DNA expressing SIVmac239 Gag and boosted on day 28 with 10⁹ vp rAd5-Gag, rAd35-Gag, rAd26-Gag, or rAd48-Gag. As shown in FIG. 6C, boosting with rAd5-Gag expanded mean tetramer⁺CD8⁺ T lymphocytes responses to 23.4% on day 35, which declined to 11.0% on day 56. Boosting with rAd26-Gag expanded mean tetramer⁺CD8⁺ T lymphocytes responses to 14.9% on day 35, which declined to 6.2% on day 56. In contrast, boosting with rAd35-Gag and rAd48-Gag elicited a lesser immune response than that observed with rAd5-Gag and rAd26-Gag. These data are consistent with the relative hierarchy of rAd vector immunogenicity observed in FIG. 4D-E. These data also suggest that prime-boost regimens consisting of two heterologous rAd vectors are comparably immunogenic to regimens consisting of DNA priming and rAd boosting.

All in all, these data indicate that if a rare serotype were preferred over a common serotype (such as Ad5), a replication-defective recombinant vector based on adenovirus serotype 26 would be an excellent serotype of choice.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth.

All publications and patent applications mentioned in this specification are herein incorporated by reference to the same extent as if each independent publication or patent application was specifically and individually indicated to be incorporated by reference.

1. An isolated nucleic acid having at least 90% sequence identity to the sequence set forth in SEQ ID NO:1, wherein said nucleic acid comprises structural and non-structural elements of an adenovirus serotype 26 (Ad26).

2. An isolated nucleic acid according to claim 1, wherein said nucleic acid has a deletion in- or of the E1 region, said deletion rendering the nucleic acid substantially replication-defective.

3. An isolated nucleic acid according to claim 1 or 2, wherein said nucleic acid has a deletion in- or of the E3 region.

4. An isolated nucleic acid according to any one of claims 1-3, wherein said nucleic acid further comprises a sequence encoding the E4orf6 gene product of an adenovirus of subgroup C.

5. An isolated nucleic acid according to claim 4, wherein said subgroup C adenovirus is adenovirus serotype 5 (Ad5).

6. An isolated nucleic acid according to claim 4 or 5, wherein said Ad5 E4orf6 coding sequence replaces the equivalent E4orf6 coding sequence of Ad26.

7. An isolated nucleic acid according to claim 2, further comprising a heterologous gene of interest.

8. An isolated nuclei acid according to claim 7, wherein said heterologous gene of interest is cloned into the region of the E1 deletion.

9. An isolated nucleic acid according to claim 7 or 8, wherein said heterologous gene of interest is under the control of a promoter.

10. An isolated nucleic acid according to any one of claims 7 to 9, wherein said heterologous gene of interest encodes a protein selected from the group consisting of: a viral protein, an antigenic determinant of a pathogenic organism, a tumor-specific antigen, a human protein, and a cytokine.

11. An isolated nucleic acid according to claim 10, wherein said heterologous gene of interest encodes a viral protein, wherein said viral protein elicits an immune response in a host.

12. An isolated nucleic acid according to claim 11, wherein said viral protein is a protein of Human Immunodeficiency Virus (HIV).

13. A recombinant replication-defective adenovirus based on Ad26, comprising a nucleic acid according to any one of claims 2-12.

14. A two-plasmid system, together comprising a nucleic acid according to any one of claims 2-12, wherein said two plasmids each contain part of the entire sequence including an overlapping sequence, which allows homologous recombination between said two plasmids resulting in a full length nucleic acid.

15. A method of producing a recombinant adenovirus according to claim 13, comprising culturing packaging cells in a suitable medium; transfecting said packaging cells with an isolated nucleic acid according to any one of claims 2-12; allowing replication of said nucleic acid in said packaging cells; and harvesting produced recombinant adenovirus from said medium and/or said cells.

16. A pharmaceutical composition comprising a recombinant adenovirus according to claim 13, and a suitable excipient.

17. A recombinant replication-defective adenovirus according to claim 13 for use as a medicament.

18. Use of a recombinant replication-defective adenovirus according to claim 13 in the manufacture of a medicament for the therapeutic, prophylactic or diagnostic treatment of an infectious disease.

19. Use according to claim 18, wherein said infectious disease is selected from the group consisting of: AIDS, malaria, ebola-infections, and tuberculosis.

20. Method of treating a host in need of treatment or in need of vaccination, comprising administering to said host a recombinant replication-defective adenovirus according to claim 13, or a pharmaceutical composition according to claim 16.

21. An isolated nucleic acid having at least 90% sequence identity to the sequence set forth in SEQ ID NO:2, wherein said nucleic acid comprises structural and non-structural elements of an adenovirus serotype 48 (Ad48).

22. An isolated nucleic acid according to claim 21, wherein said nucleic acid has a deletion in- or of the E1 region, said deletion rendering the nucleic acid substantially replication-defective.

23. An isolated nucleic acid according to claim 21 or 22, wherein said nucleic acid has a deletion in- or of the E3 region.

24. An isolated nucleic acid according to any one of claims 21-23, wherein said nucleic acid further comprises a sequence encoding the E4orf6 gene product of an adenovirus of subgroup C.

25. An isolated nucleic acid according to claim 24, wherein said subgroup C adenovirus is adenovirus serotype 5 (Ad5).

26. An isolated nucleic acid according to claim 24 or 25, wherein said Ad5 E4orf6 coding sequence replaces the equivalent E4orf6 coding sequence of Ad48.

27. An isolated nucleic acid according to claim 22, further comprising a heterologous gene of interest.

28. An isolated nuclei acid according to claim 27, wherein said heterologous gene of interest is cloned into the region of the E1 deletion.

29. An isolated nucleic acid according to claim 27 or 28, wherein said heterologous gene of interest is under the control of a promoter.

30. An isolated nucleic acid according to any one of claims 27 to 29, wherein said heterologous gene of interest encodes a protein selected from the group consisting of: a viral protein, an antigenic determinant of a pathogenic organism, a tumor-specific antigen, a human protein, and a cytokine.

31. An isolated nucleic acid according to claim 30, wherein said heterologous gene of interest encodes a viral protein, wherein said viral protein elicits an immune response in a host.

32. An isolated nucleic acid according to claim 31, wherein said viral protein is a protein of Human Immunodeficiency Virus (HIV).

33. A recombinant replication-defective adenovirus based on Ad48, comprising a nucleic acid according to any one of claims 22-32.

34. A two-plasmid system, together comprising a nucleic acid according to any one of claims 22-32, wherein said two plasmids each contain part of the entire sequence including an overlapping sequence, which allows homologous recombination between said two plasmids resulting in a full length nucleic acid.

35. A method of producing a recombinant adenovirus according to claim 33, comprising culturing packaging cells in a suitable medium; transfecting said packaging cells with an isolated nucleic acid according to any one of claims 22-32; allowing replication of said nucleic acid in said packaging cells; and harvesting produced recombinant adenovirus from said medium and/or said cells.

36. A pharmaceutical composition comprising a recombinant adenovirus according to claim 33, and a suitable excipient.

37. A recombinant replication-defective adenovirus according to claim 33 for use as a medicament.

38. Use of a recombinant replication-defective adenovirus according to claim 33 in the manufacture of a medicament for the therapeutic, prophylactic or diagnostic treatment of an infectious disease.

39. Use according to claim 38, wherein said infectious disease is selected from the group consisting of: AIDS, malaria, ebola-infections, and tuberculosis.

40. Method of treating a host in need of treatment or in need of vaccination, comprising administering to said host a recombinant replication-defective adenovirus according to claim 33, or a pharmaceutical composition according to claim 36.


SEQUENCE LISTING (45 sequences)

SEQ ID NO: 1 - DNA, 35155 bp, Human Adenovirus serotype 26
catcatcaataatataccccacaaagtaaacaaaagttaatatgcaaatgagcttttgaattttaacggttttggggcggagccaacgctgattggacgagaaacggtgatgcaaatgacgtcacgacgcacggctaacggtcgccgcggaggcgtggcctagcccggaagcaagtcgcggggctgatgacgtataaaaaagcggactttagacccggaaacggccgattttcccgcggccacgcccggatatgaggtaattctgggcggatgcaagtgaaattaggtcattttggcgcgaaaactgaatgaggaagtgaaaagcgaaaaataccggtccctcccagggcggaatatttaccgagggccgagagactttgaccgattacgtgggggtttcgattgcggtgtttttttcgcgaatttccgcgtccgtgtcaaagtccggtgtttatgtcacagatcagctgatccgcagggtatttaaaccagtcgagtccgtcaagaggccactcttgagtgccagcgagtagagatttctctgagctccgctcccagagaccgagaaaaatgagacacctgcgcctcctgccttcaactgtgcccggtgagctggctgtgcttatgctggaggactttgtggatacagtattggaggacgaactgcatccaagtccgttcgagctgggacccacacttcaggatctctatgatctggaggtagatgcccatgatgacgaccctaacgaggaggctgtgaatttaatatttccagaatctatgattcttcaggctgacatagccaacgaatctactccacttcatacaccgactctgtcacccatacctgaattggaagaggaggacgaactagacctccggtgttatgaggaaggttttcctcccagcgattcagaggatgaacggggtgagcagaccatggctctgatctcagactatgcttgtgtgattgtggaggaacaagtagtgattgaaaattctaccgagccagtggagggctgtagaaaatgccagtaccaccgggataagtctggagacccgaacgcatcatgcgctttgtgctatatgaaacagactttcagctttatttacagtaagtggagtgaatgtgagagaggctgagtgcttaacacatcactgtgtattgcttgaacagctgtgctaagtgtggtttatttttgtttctaggtccggtgtcagaggatgagtcatcaccctcagaagaagaccacccgtctccccctgatctcacagatgacacgcccctgcaagtgcacagacccaccccagtcagagccagtggcgagaggcgagcagctgttgaaaaaattgaggacttgttacatgacatgggtggggatgaacctttggacctgagcttgaaacgccccaggaactaggcgcagctgcgcttagtcatgtgtaaataaagttgtacaataaaagtatatgtgacgcatgcaaggtgtggtttatgactcatgggcggggcttagtcctatataagtggcaacacctgggcactgggcacagaccttcagggagttcctgatggatgtgtggactatccttgcagactttagcaagacacgccggcttgtagaggatagttcagacgggtgctccgggttctggagacactggtttggaactcctctatctcgcctggtgtacacagttaagaaggattataaagaggaatttgaaaatatttttgctgactgctctggcctgctagattctctgaatcttggccaccagtcccttttccaggaaagggtactccacagccttgatttttccagcccagggcgcactacagccggggttgcttttgtggtttttctggttgacaaatggagccaggacacccaactgagcaggggctacatcctggacttcgcagccatgcacctgtggagggcctggatcaggcagcggggacagagaatcttgaattactggcttctacagccagcagctccgggtcttcttcgtctacacagacaaacatccatgttggaggaagaaatgaggcaggccatggacgagaacccgaggagcggcctggaccctccgtcggaagaggagctggattgaatcaggtatccagcctgtacccagagcttagcaaggtgctgacatccatggccaggggagttaagagggagaggagcgatgggggtaataccgggatgatgaccgagctgacggccagcctgatgaatcggaagcgcccagagcgccttacctggtacgagctacagcaggagtgcagggatgagttgggcctgatgcaggataaatatggcctggagcagataaaaacccattggttgaacccagatgaggattgggaggaggctattaagaagtatgccaagatagccctgcgcccagattgcaagtacatagtgaccaagaccgtgaatatcagacatgcctgctacatctcggggaacggggcagaggtggtcatcgataccctggacaaggccgccttcaggtgttgcatgatgggaatgagagcaggagtgatgaatatgaattccatgatcttcatgaacatgaagttcaatggagagaagtttaatggggtgctgttcatggccaacagccacatgaccctgcatggctgcagtttcttcggcttcaacaatatgtgcgcagaggtctggggcgcttccaagatcaggggatgtaagttttatggctgctggatgggcgtggtcggaagacccaagagcgagatgtctgtgaagcagtgtgtgtttgagaaatgctacctgggagtctctaccgagggcaatgctagagtgagacactgctcttccctggagacgggctgcttctgcctggtgaagggcacagcctctctgaagcataatatggtgaagggctgcacggatgagcgcatgtacaacatgctgacctgcgattcgggggtctgccatatcctgaagaacatccatgtgacctcccaccccagaaagaagtggccagtgtttgagaataacctgctgatcaagtgccatatgcacctgggagccagaaggggcaccttccagccgtaccagtgcaactttagccagaccaagctgctgttggagaacgatgccttctccagggtgaacctgaacggcatctttgacatggatgtctcggtgtacaagatcctgagatacgatgagaccaagtccagggtgcgcgcttgcgagtgcgggggcagacacaccaggatgcagccagtggccctggatgtgaccgaggagctgagaccagaccacctggtgatggcctgtaccgggaccgagttcagctccagtggggaggatacagattagaggtaggtttgagtagtgggcgtggctaaggtgactataaaggcgggtgtcttacgagggtctttttgcttttctgcagacatcatgaacgggactggcggggccttcgaaggggggctttttagcccttatttgacaacccgcctgccgggatgggccggagttcgtcagaatgtgatgggatcgacggtggatgggcgcccagtgcttccagcaaattcctcgaccatgacctacgcgaccgtggggaactcgtcgctcgacagcaccgccgcagccgcggcagccgcagccgccatgacagcgacgagactggcctcgagctacatgcccagcagcggtagtagcccctctgtgcccagttccatcatcgccgaggagaaactgctggccctgctggccgagctggaagccctgagccgccagctggccgccctgacccagcaggtgtccgagctccgcgaacagcagcagcagcaaaataaatgattcaataaacacagattctgattcaaacagcaaagcatctttattatttattttttcgcgcgcggtaggccctggtccacctctcccgatcattgagagtgcggtggattttttccaggacccggtagaggtgggattggatgttgaggtacatgggcatgagcccgtcccgtgggtggaggtagcaccactgcatggcctcgtgctctggggtcgtgttgtagatgatccagtcatagcaggggcgctgggcgtggtgctggatgatgtccttgaggaggagactgatggccacggggagccccttggtgtaggtgttggcaaaacggttgagctgggagggatgcatgcggggggagatgatgtgcagtttggcctggatcttgaggttggcgatgttgccacccagatcccgccgggggttcatgttgtgcaggaccaccagaacggtgtagcccgtgcacttggggaacttgtcatgcaacttggaagggaatgcgtggaagaatttggagacgcccttgtgcccgcccaggttttccatgcactcatccatgatgatggcaatgggcccgtgggctgcggctttggcaaagacgtttctggggtcagagacatcgtaattatgctcctgggtgagatcatcataagacattttaatgaatttggggcggagggtgccagattgggggacgatggttccctcgggccccggggcgaagttcccctcgcagatctgcatctcccaggctttcatctcggagggggggatcatgtccacctgcggggcgatgaaaaaaacggtttccggggcgggggtgatgagctgcgaggagagcaggtttctcaacagctgggacttgccgcacccggtcgggccgtagatgaccccgatgacgggttgcaggtggtagttcaaggacatgcagctgccgtcgtcccggaggaggggggccacctcgttgagcttgtctctgacttggaggttttcccggacgagctcgccgaggaggcggtccccgcccagcgagagaagctcttgcagggaagcaaagtttttcaggggcttgagcccgtcggccatgggcatcttggcgagggtctgcgagaggagctccaggcggtcccagagctcggtgacgtgctctacggcatctcgatccagcagacttcctcgtttcgggggttgggacgactgcgactgtagggcacgagacgatgggcgtccagcgcggccagcgtcatgtccttccagggtctcagggtccgcgtgagggtggtctccgtcacggtgaaggggtgggccgcgggctgggcgcttgcaagggtgcgcttgagactcatcctgctggtgctgaaacgggcacggtcttcgccctgcgcgtcggcgagatagcagttgaccatgagctcgtagttgagggcctcggcggcgtggcccttggcgcggagcttgcccttggaagagcgcccgcaggcgggacagaggagggattgcagggcgtagagcttgggcgcgagaaagacggactcgggggcgaaggcgtccgctccgcagtgggcgcagacggtctcgcactcgactagccaggtgagctcgggctgctcggggtcaaaaaccagttttcccccgttctttttgatgcgcttcttacctcgcgtctccatgagtctgtgtccgcgctcggtgacaaacaggctgtctgtgtccccgtagacggacttgatgggcctgtcctgcaggggcgtcccgcggtcctcctcgtagagaaactcagaccactctgagacgaaggcgcgcgtccacgccaagacaaaggaggccacgtgcgaggggtagcggtcgttgtccaccagggggtccaccttttccacggtatgcaggcacatgtccccctcctccgcatccaagaaggtgattggcttgtaggtgtaggccacgtgacctggggttcccgacgggggggtataaaagggggcgggtctgtgctcgtcctcactctcttccgcgtcgctgtccacgagcgccagctgttggggtaggtattccctctcaagagcgggcatgacctcggcactcaggttgtcagtttctagaaacgaggaggatttgatgtgggcctgccctgccgcgatgctttttaggagactttcatccatctggtcagaaaagactatttttttattgtcaagcttggtggcgaaggagccatagagggcgtttgagagaagcttggcgatggatctcatggtctgatttttgtcacggtcggcgcgctccttggccgcgatgttgagctggacatattcgcgcgcgacacacttccattcggggaagacggtggtgcgctcgtcgggcacgatcctgacgcgccagccgcggttatgcagggtgaccaggtccacgctggtggccacctcgccgcgcaggggctcgttggtccagcagagtctgccgcccttgcgcgagcagaacgggggcagcacatcaagcagatgctcgtcaggggggtccgcatcgatggtgaagatgcccggacagagttccttgtcaaaataatcgatttttgaggatgcatcgtccaaggccatctgccactcgcgggcggccagcgctcgctcgtaggggttgaggggcggaccccaaggcatgggatgcgtgagggcggaggcgtacatgccgcagatgtcatagacatagatgggctccgagaggatgccgatgtaggtgggatagcagcgccccccgcggatgcttgcgcgcacgtagtcatacaactcgtgcgagggggccaagaaggcggggccgagattggtgcgctggggctgctcggcgcggaagacgatctggcgaaagatggcgtgcgagttggaggagatggtgggccgttggaagatgttaaagtgggcgtgaggcaggcggaccgagtcgcggatgaagtgcgcgtaggagtcttgcagcttggcgacgagctcggcggtgacgaggacgtccatggcgcagtagtccagcgtttcgcggatgatgtcataactcgcctctcctttcttctcccacagctcgcggttgagggcgtattcctcgtcatccttccagtactcccggagcgggaatcctcgatcgtccgcacggtaagagcccagcatgtagaaatggttcacggccttgtagggacagcagcccttctccacggggagggcgtaagcttgagcggccttgcggagcgaggtgtgcgtcagggcaaaggtgtccctgaccatgactttcaagaactggtacttgaagtccgagtcgtcgcagccgccgtgctcccagagctcgaaatcggtgcgcttcttcgagagggggttaggcagagcgaaagtgacgtcattgaagagaatcttgcctgcccgcggcatgaaattgcgggtgatgcggaaagggcccgggacggaggctcggttgttgatgacctgggcggcgaggacgatctcgtcaaagccgttgatgttgtgcccgacgatgtagagttccatgaatcgcgggcggcctttgatgtgcggcagctttttgagctcctcgtaggtgaggtcctcggggcattgcaggccgtgctgctcgagcgcccactcctggagatgtgggttggcttgcatgaaggaagcccagagctcgcgggccatgagggtctggagctcgtcgcgaaagaggcggaactgctggcccacggccatcttttctggggtgacgcagtagaaggtgagggggtcccgctcccagcgatcccagcgtaaacgcacggcgagatcgcgagcgagggcgaccagctctgggtccccggagaatttcatgaccagcatgaaggggacgagctgcttgccgaaggaccccatccaggtgtaggtttctacatcgtaggtgacaaagagccgctccgtgcgaggatgagagccgattgggaagaactggatttcctgccaccagttggacgagtggctgttgatgtgatgaaagtagaaatcccgccggcgaaccgagcactcgtgctgatgcttgtaaaagcgtccgcagtactcgcagcgctgcacgggctgtacctcatccacgagatacacagcgcgtcccttgaggaggaacttcaggagtggcggccctggctggtggttttcatgttcgcctgcgtgggactcaccctggggctcctcgaggacggagaggctgacgagcccgcgcgggagccaggtccagatctcggcgcggcgggggcggagagcgaagacgagggcgcgcagttgggagctgtccatggtgtcgcggagatccaggtccgggggcagggttctgaggttgacctcgtagaggcgggtgagggcgtgcttgagatgcagatggtacttgatttctacgggtgagttggtggtcgtgtccacgcattgcatgagcccgtagctgcgcggggccacgaccgtgccgcggtgcgcttttagaagcggtgtcgcggacgcgctcccggcggcagcggcggttccggccccgcgggcaggggcggcagaggcacgtcggcgtggcgctcgggcaggtcccggtgctgcgccctgagagcgctggcgtgcgcgacgacgcggcggttgacatcctggatctgccgcctctgcgtgaagaccacgggccccgtgactttgaacctgaaagacagttcaacagaatcaatctctgcgtcattgacggcggcctgacgcaggatctcttgcacgtcgcccgagttgtcctggtaggcgatctcggacatgaactgttcgatctcctcctcctggagatcgccgcggcccgcgcgctccacggtggcggcgaggtcattggagatgcgacccatgagctgcgagaaggcgcccaggccgctctcgttccagacgcggctgtagaccacgtccccgtcggcgtcgcgcgcgcgcatgaccacctgcgcgaggttgagctccacgtgccgcgcaaagacggcgtagttgcgcaggcgctggaagaggtagttgagggtggtggcgatgtgctcggtgacgaagaagtacatgatccagcggcgcaggggcatctcgctgatgtcgccgatggcttccagcctttccatggcctcgtagaagtccacggcgaagttgaaaaactgggcgttgcgggccgagaccgtgagctcgtcttccaggagccggatgagttcggcgatggtggcgcgcacctcgcgctcgaaatccccgggggcctcctcctcttcctcttcttccatgacgacctcttcttctatttcttcctctgggggcggtggtggtggcgggggccgacgacgacggcgacgcaccgggagacggtcgacgaagcgctcgatcatctccccgcggcggcgacgcatggtttcggtgacggcgcgaccccgttcgcgaggacgcagcgtgaagacgccgccggtcatctcccggtaatggggcgggtccccattgggcagcgatagggcgctgacgatgcatcttatcaattgcggtgtaggggacgtgagcgcgtcgagatcgaccggatcggagaatctttcgaggaaagcgtctagccaatcgcagtcgcaaggtaagctcaaacacgtagcagccctgcggacgctgttagaattgcggttgctgatgatgtaattgaagtaggcgtttttgaggcggcggatggtggcgaggaggaccaggtccttgggtccagcttgctggatgcggagccgctcggccatgccccaggcctggccctgacaccggctcaggttcttgtagtagtcatgcatgagcctctcaatgtcatcactggctgaggcggagtcttccatgcgggtgaccccgacgcccctgagcggctgcacgagcgccaggtcggcgacgacgcgctcggcgaggatggcctgttgcacgcgggtgagggtgtcctggaagtcgtccatgtcgacgaagcggtgataggccccggtgttgatggtgtaggtgcagttggccatgagcgaccagttgacggtctgcaggcctggctgcacgacctcggagtacctgagccgcgagaaggcgcgcgagtcgaagacgtagtcgttgcaggtgcgcacgaggtactggtatccgactaggaagtgcggcggcggctggcggtagagcggccagcgctgggtggccggcgcgcccggggccaggtcctcgagcatgaggcggtggtagccgtagaggtagcgggacatccaggtgatgccggcggcggtggtggaggcgcgcgggaactcgcggacgcggttccagatgttgcgcagcggcaggaaatagtccatggtcggcacggtctggccggtgagacgcgcgcagtcattgacgctctagaggcaaaaacgaaagcggttgagcgggctcttcctccgtagcctggcggaacgcaaacgggttaggccgcgtgtgtaccccggttcgagtcccctcgaatcaggctggagccgcgactaacgtggtattggcactcccgtctcgacccgagcccgatagccgccaggatacggcggagagccctttttgccggccgagtggggtcgctagacttgaaagcgaccgaaaaccccgccgggtagtggctcgcgcccgtagtctggagaagcatcgccagggttgagtcgcggcagaacccggttcgaggacggccgcggcgagcgggacttggtcaccccgccgatataaagacccacagccagccgacttctccagttacgggagcgagcccccttttttctttttgccagatgcatcccgtcctgcgccaaatgcgtcccacccccccggcgaccaccgcgaccgcggccgtagcaggcgccggcgctagccagccaccacagacagagatggacttggaagagggcgaagggctggcaagactgggggcgccgtccccggagcgacatccccgcgtgcagctgcagaaggacgtgcgcccggcgtacgtgcctacgcagaacctgttcagggaccgcagcggggaggagcccgaggagatgcgcgactgccggtttcgggcgggcagggagctgcgcgagggcctggaccgccagcgcgtgctgcgcgacgaggatttcgagccgaacgagcagacggggatcagccccgcacgcgcgcacgtggcggcagccaacctggtgacggcctacgagcagacggtgaagcaggagcgcaacttccaaaagagtttcaacaaccacgtgcgcaccctgatcgcgcgcgaggaggtggccctgggcctgatgcacctgtgggacctggcggaggccatcgtgcagaacccggacagcaagcctctgacggcgcagctgttcctggtggtgcagcacagcagggacaacgaggcgttcagggaggcgctgctgaacatcgccgagcccgagggtcgctggctgctggagctgattaacatcttgcagagcatcgtagtgcaggagcgcagcctgagcctggccgagaaggtggcggcgatcaactactcggtgctgagcctgggcaagttttacgcgcgcaagatttacaagacgccgtacgtgcccatagacaaggaggtgaagatagacagcttttacatgcgcatggcgctcaaggtgctgacgctgagcgacgaccttggcgtgtaccgcaacgaccgcatccacaaggccgtgagcacgagccggcggcgcgagctgagcgaccgcgagctgatgctgagtctgcgccgggcgctggtagggggcgccgccggcggcgaggagtcctacttcgacatgggtgcggacctgcattggcagccgagccggcgcgccttggaggccgcctacggttcagaggacttggatgaggaagaggaagaggaggaggatgcacccgctgcggggtactgacgcctccgtgatgtgtttttagatgtcccagcaagccccggaccccgccataagggcggcgctgcaaagccagccgtccggtctagcatcggacgactgggaggccgcgatgcaacgcatcatggccctgacgacccgcaaccccgagtcctttagacaacagccgcaggccaacagactctcggccattctggaggcggtggtcccctctcggaccaaccccacgcacgagaaggtgctggcgatcgtgaacgcgctggcggagaacaaggccatccgtcccgacgaggccgggctggtgtacaacgccctgctggagcgcgtgggccgctacaacagcacgaacgtgcagtccaacctggatcggctggtgacggacgtgcgcgaggccgtggcgcagcgcgagcggttcaagaacgagggcctgggctcgctggtggcgctgaacgccttcctggcaacgcagccggcgaacgtgccgcgcgggcaggacgattacaccaactttatcagcgcgctgcggctgatggtgaccgaggtgccccagagcgaggtgtaccagtctggcccggactactttttccagacgagccggcagggcttgcagacggtgaacctgagccaggctttcaagaatctgcgcgggctgtggggcgtgcaggcgcccgtgggcgaccggtcaacggtgagcagcttgctgacgcccaactcgcggctgctgctgctgctgatcgcgcccttcaccgacagcggcagcgtgaaccgcaactcgtacctgggccatctgctgacgctgtaccgcgaggccataggccaggcgcaggtggacgagcagaccttccaggagatcactagcgtgagccgcgcgctggggcagaacgacaccgacagtctgagggccaccctgaactttttgctgaccaatagacagcagaagatcccggcgcagtacgcactgtcggccgaggaggaaaggattctgagatatgtgcagcagagcgtagggctgttcctgatgcaggagggtgccacccccagcgccgcgctggacatgaccgcgcgcaacatggaacctagcatgtacgccgccaaccggccgttcatcaataagctgatggactacttgcaccgcgcggcggccatgaacacggactactttaccaacgccatcctgaacccgcactggctcccgccgccggggttctacacgggcgagtacgacatgcccgaccccaacgacgggttcctgtgggacgacgtggacagcgcggtgttctcgccgacctttcaaaagcgccaggaggcgccgccgagcgagggcgcggtggggaggagcccctttcctagcttagggagtttgcatagcttgccgggctcggtgaacagcggcagggtgagccggccgcgcttgctgggcgaggacgagtacctgaacgactcgctgctgcagccgccgcgggccaagaacgccatggccaataacgggatagagagtctggtggacaaactgaaccgctggaagacctacgctcaggaccatagggacgcgcccgcgccgcggcgacagcgccacgaccggcagcggggcctggtgtgggacgacgaggactcggccgacgatagcagcgtgttggacttgggcgggagcggtggggtcaacccgttcgcgcatctgcagcccaaactggggcgacggatgttttgaatgaaataaaactcaccaaggccatagcgtgcgttctcttccttgttagagatgaggcgcgcggtggtgtcttcctctcctcctccctcgtacgagagcgtgatggcgcaggcgaccctggaggttccgtttgtgcctccgcggtatatggctcctacggagggcagaaacagcattcgttactcggagctggctccgcagtacgacaccactcgcgtgtacttggtggacaacaagtcggcggacatcgcttccctgaactaccaaaacgaccacagcaacttcctgaccacggtggtgcagaacaacgatttcacccccgccgaggccagcacgcagacgataaattttgacgagcggtcgcggtggggcggtgatctgaagaccattctgcacactaacatgcccaatgtgaacgagtacatgttcaccagcaagtttaaggcgcgggtgatggtgtctaggaagcatccagagggggtagttgaaacagatttgagtcaggataagcttgaatatgagtggtttgagtttaccctgcccgagggaaacttttccgagaccatgaccatagacctgatgaacaacgccatcttggaaaactacttgcaagtggggcggcagaatggcgtgctggagagcgatatcggagtcaagtttgacagcagaaatttcaagctgggctgggacccggtgaccaagctggtgatgccaggggtctacacctacgaggccttccacccggacgtggtgctgctgccgggctgcggggtggacttcaccgagagccgcctgagcaacctcctgggcattcgcaagaagcaacctttccaagagggcttcagaatcatgtatgaggatctagaaggtggcaacatccccgccctccttgatgtgcccaagtacttggaaagcaagaagaaagttgaagacgaaactaaaaatgcagctgcggccacagccgatacaaccactaggggtgatacatttgcaactccagcgcaagagacagcagctgataagaaggtagaagtcttgcccattgaaaaggatgagagtggtagaagttacaacctgatccaggggacccacgacacgctgtaccgcagttggtacctgtcctatacctacggggaccccgagaagggggtgcagtcgtggacgctgctcaccaccccggacgttacctgcggcgcggagcaagtctactggtcactgccggacctcatgcaagaccccgtcaccttccgctccacccagcaagtcagcaactaccccgtggtcggcgccgagctcatgcccttccgcgccaagagcttttacaacgacctcgccgtctactcccagctcatccgcagctacacctccctcacccacgtcttcaaccgcttccccgacaaccagatcctctgccgcccgcccgcgcccaccatcaccaccgtcagtgaaaacgtgcctgctctcacagatcacgggacgctaccgctgcgcagcagtatccgcggagtccagcgagtgaccgtcactgacgcccgtcgccgcacctgtccctacgtctacaaggccctgggcatagtcgcgccgcgcgtgctttccagtcgcaccttctaaaaaaatgtctattctcatctcgcccagcaataacaccggctggggtcttactagacccagcaccatgtacggaggagccaagaagcgctcccagcagcaccccgtccgcgtccgcggccacttccgcgctccctggggcgcttacaagcgcgggcggacttccaccgccgccgtgcgcaccaccgtcgacgacgtcatcgactcggtggtcgccgacgcgcgcaactacactcccgccccctccaccgtggacgcggtcatcgacagcgtggtggccgacgcgcgcgactatgccagacgcaagagccggcggcgacggatcgccaggcgccaccggagcacgcccgccatgcgcgccgcccgggctctgctgcgccgcgccagacgcacgggccgccgggccatgatgcgagccgcgcgccgcgctgccactgcacccacccccgcaggcaggactcgcagacgagcggccgccgccgccgctgcggccatctctagcatgaccagacccaggcgcggaaacgtgtactgggtgcgcgactccgtcacgggcgtgcgcgtgcccgtgcgcacccgtcctcctcgtccctgatctaatgcttgtgtcctcccccgcaagcgacgatgtcaaagcgcaaaatcaaggaggagatgctccaggtcgtcgccccggagatttacggaccaccccaggcggaccagaaaccccgcaaaatcaagcgggttaaaaaaaaggatgaggtggacgagggggcagtagagtttgtgcgcgagttcgctccgcggcggcgcgtaaattggaaggggcgcagggtgcagcgcgtgttgcggcccggcacggcggtggtgttcacgcccggcgagcggtcctcggtcaggagcaagcgtagctatgacgaggtgtacggcgacgacgacatcctggaccaggcggcggagcgggcgggcgagttcgcctacgggaagcggtcgcgcgaagaggagctgatctcgctgccgctggacgaaagcaaccccacgccgagcctgaagcccgtgaccctgcagcaggtgctgccccaggcggtgctgctgccgagccgcggggtcaagcgcgagggcgagagcatgtacccgaccatgcagatcatggtgcccaagcgccggcgcgtggaggacgtgctggacaccgtgaaaatggatgtggagcccgaggtcaaggtgcgccccatcaagcaggtggcgccgggcctgggcgtgcaaaccgtggacattcagatccccaccgacatggatgtcgacaaaaaaccctcgaccagcatcgaggtgcaaaccgacccctggctcccagcctccaccgctaccgtctccacttctaccgccgccacggctaccgagcctcccaggaggcgaagatggggcgccgccagccggctgatgcccaactacgtgttgcatccttccatcatcccgacgccgggctaccgcggcacccggtactacgccagccgccggcgcccagccagcaaacgccgccgccgcaccgccacccgccgccgtctggcccccgcccgcgtgcgccgcgtgaccacgcgccggggccgctcgctcgttctgcccaccgtgcgctaccaccccagcatcctttaattcgtgtgctgtgatactgttgcagagagatggctctcacttgccgcctgcgcatccccgtcccgaattaccgaggaagatcccgccgcaggagaggcatggcaggcagcggcctgaaccgccgccggcggcgggccatgcgcaggcgcctgagtggcggctttctgcccgcgctcatccccataatcgccgcggccatcggcacgatcccgggcatagcttccgttgcgctgcaggcgtcgcagcgccgttgatgtgcgaataaagcctctttagactctgacacacctggtcctgtatatttttagaatggaagacatcaattttgcgtccctggctccgcggcacggcacgcggccgttcatgggcacctggaacgagatcggcaccagccagctgaacgggggcgccttcaattggagcagtgtctggagcgggcttaaaaatttcggctcgacgctccggacctatgggaacaaggcctggaatagtagcacggggcagttgttgagggaaaagctcaaagaccaaaacttccagcagaaggtggtggacgggctggcctcgggcattaacggggtggtggacatcgcgaaccaggccgtgcagcgcgagataaacagccgcctggacccgcgaccgcccacggtggtggagatggaagatgcaactcttccgccgcccaaaggcgaaaagcggccgcggcccgacgcggaggagacgatcctgcaggtggacgagccgccctcgtacgaggaggccgtcaaggccggcatgcccaccacgcgcatcatcgcgccgctggccacgggtgtaatgaaacccgccacccttgacctgcctccaccacccgcgcccgctccaccgaaggcaactccggttgtgcaggcccccccggtggcgaccgccgtgcgccgcgtccccgcccgccgccaggcccagaactggcagagcacgctgcacagtatcgtgggcctgggagtgaaaagtctgaagcgccgccgatgctattgagagagaggaaagaggacactaaagggagagcttaacttgtatgtgccttaccgccagagaacgcgcgaagatggccaccccctcgatgatgccgcagtgggcgtacatgcacatcgccgggcaggacgcctcggagtacctgagcccgggtctggtgcagtttgcccgcgccaccgacacgtacttcagcctgggcaacaagtttaggaaccccacggtggccccgacccacgatgtgaccacggaccggtcccagcgtctgacgctgcgcttcgtgcccgtggatcgcgaggacaccacgtactcgtacaaggcgcgcttcactctggccgtgggcgacaaccgggtgctagacatggccagcacttactttgacatccgcggcgtcctggaccgcggtcccagcttcaaaccctactcgggcacggcctacaacagcctggctcccaagggtgcccccaatcccagtcagtgggaaacaaaagaaaagcaaggaactactggaggagtgcagcaagaaaaagatgtcacaaaaacatttggtgtggctgccaccggcggaattaatataacaaaccagggtctgttactaggaactgacgaaaccgctgagaatggcaaaaaagacatttatgcagacaagactttccagccagaacctcaagttggagaagaaaactggcaggaaaatgaagccttctatggaggaagggctcttaaaaaggacactaaaatgaaaccatgctatggatcttttgctagacctactaatgagaaaggaggtcaggcaaagttcaaaccagttaatgaaggagaacaacctaaagatctggatatagattttgcttactttgacgtccctggcggaagtcctccagcaggtggtagtggggaagaatacaaagcagatataattttgtacactgaaaatgttaatcttgaaacaccagacactcatgtggtttacaagccaggaacttcagataacagttcagaaatcaatctggttcagcagtccatgccaaacagacccaactacattggctttagggacaactttgtaggtctcatgtattacaacagcaccggaaatatgggtgtgctggctggtcaggcttctcagttgaacgctgtggtcgacttgcaagacagaaacaccgagttatcttaccagctattgctagattctctgggtgacagaaccagatactttagcatgtggaactctgcggtggacagttacgatccagatgtcaggatcattgaaaatcacggtgtggaagatgaacttccaaactattgcttcccattgaatggcactggaaccaattccacttatcaaggtgtaaagattacaaatggtaatgatggtgctgaagaaagtgagtgggagaaagacgatgcaatttctagacaaaaccaaatctgcaagggcaatgtctacgccatggagatcaacctgcaggccaacctgtggaagagttttctgtactcgaacgtggccctgtacctgcccgactcctacaagtacacgccggccaacgtcaagctgcccgccaacaccaacacctacgagtacatgaacggccgcgtggtagccccatccctggtggacgcctacatcaacatcggcgcccgctggtcgttggaccccatggacaacgtcaaccccttcaaccaccaccgcaatgcgggcctgcgctaccgctccatgctgctgggcaacggccgctacgtgcccttccacatccaagtgccccaaaagttctttgccatcaagaacctgctcctgctcccgggctcctacacctacgagtggaacttccgcaaggacgtcaacatgatcctgcagagttccctcggcaacgacctgcgcgtcgacggcgcctccgtccgcttcgacagcgtcaacctatacgccactttcttccccatggcgcacaacaccgcttcaaccttggaagccatgctgcgcaacgacaccaacgaccagtccttcaacgactacctctcggccgccaacatgctctaccccatcccggccaaggccaccaacgtgcccatctccatcccatcgcgcaactgggccgccttccgcggctggagtttcacccggctcaagaccaaggaaactccttccctcggctcgggtttcgacccctactttgtctactcgggctccatcccctacctcgacgggaccttctacctcaaccacaccttcaagaaggtctccatcatgttcgactcctcggtcagctggcccggcaacgaccggctgctcacgccgaacgagttcgagatcaagcgcagcgtcgacggggagggctacaacgtggcccaatgcaacatgaccaaggactggttcctcgtccagatgctctcccactacaacatcggctaccagggcttccacgtgcccgagggctacaaggaccgcatgtactccttcttccgcaacttccagcccatgagcaggcaggtggtcgatgagatcaactacaaggactacaaggccgtcaccctgcccttccagcacaataactcgggcttcaccggctacctcgcacccaccatgcgccaggggcagccctaccccgccaacttcccctacccgctcatcggtcagacagccgtgccctccgtcacccagaaaaagttcctctgcgacagggtcatgtggcgcatcccattctccagcaacttcatgtccatgggcgccctcaccgacctgggtcagaacatgctctacgccaactcggcccacgcgctcgacatgaccttcgaggtggaccccatggatgagcccaccctcctctatcttctcttcgaagttttcgacgtggtcagagtacaccagccgcaccgcggcgtcatcgaggccgtctacctgcgcacgcccttctccgccggcaacgccaccacctaagcatgagcggctccagcgaacgagagctcgcggccatcgtgcgcgacctgggctgcgggccctactttttgggcacccacgacaagcgcttcccgggctttctcgccggcgacaagctggcctgcgccatcgtcaacacggccggccgcgagaccggaggcgtgcactggctcgccttcggctggaacccgcgctcgcgcacctgctacatgttcgacccctttgggttctcggaccgccggctcaagcagatttacagcttcgagtacgaggccatgctgcgccgcagcgccctggcctcctcgcccgaccgctgtctcagcctcgagcagtccactcagaccgtgcaggggcccgactccgccgcctgcggactcttctgttgcatgttcttgcatgccttcgtgcactggcccgaccgacccatggacggaaaccccaccatgaacttgctgacgggggtgcccaacggcatgctacaatcgccacaggtgctgcccaccctcaggcgcaaccaggaggaactctaccgcttcctcgcgcgccactccccttactttcgctcccaccgcgccgccatcgaacacgccaccgcttttgacaaaatgaaacaactgcgtgtatctcaataaacagcacttttattttacatgcactggagtatatgcaagttatttaaaagtcgaaggggttctcgcgctcgtcgttgtgcgccgcgctggggagggccacgttgcggtactggtacttgggctgccacttgaactcggggatcaccagtttgggcactggggtctcggggaaggtctcgctccacatgcgccggctcatctgcagggcgcccagcatgtccggggcggagatcttgaaatcgcagttggggccggtgctctgcgcgcgcgagttgcggtacacggggttgcagcactggaacaccatcagactggggtacttcacactagccagcacgctcttgtcgctgatctgatccttgtccagatcctcggcgttgctcaggccgaacggggtcatcttgcacagctggcgtcccaggaagggcacgctctgaggcttgtggttacactcgcagtgcacgggcatcagcatcatccccgcgccgcgctgcatattcgggtagagggccttgacaaaggccgcgatctgcttgaaagcttgctgggccttggccccctcgctgaaaaacaggccgcagctcttcccgctgaactggttattcccacacccggcatcctgcacgcagcagcgcgcgtcatggctggtcagttgcaccacgctccgtccccagcggttctgggtcaccttagccttgctgggctgctccttcaacgcgcgctgcccgttctcgctggtcacatccatctccaccacgtggtccttgtggatcatcatcgtcccgtgcagacacttgagctggccttccacctcggtgcagccgtgatcccacagggcgcaaccggtgcactcccagttcttgtgcgcaatcccgctgtggctgaagatgtaaccttgcaacatgcggcccatgatggtgctaaatgctttctgggtggtgaaggtcagttgcatcccgcgggcctcctcgttcatccaggtctggcacatcttctggaagatctcggtctgctcgggcatgagcttgtaagcatcgcgcaggccgctgtcgacgcggtagcgttccatcagcacgttcatggtatccatgcccttctcccaggacgagaccagaggcagactcagagggttgcgtacgttcaggacaccgggggtcgcgggctcgacgatgcgttttccgtccttgccttccttcaatagaaccggcggctggctgaatcccactcccacgatcacggcatcttcttcctggggcatctcttcgtcggggtctaccttggtcacatgcttggtctttctggcttgcttcttttttggagggctgtccacggggagcacgtcctcctcggaagacccggagcccacccgctgatactttcggcgcttggtgggcagaggaggtggcggcgaggggctcctctcctgctccggcggatagcgcgccgacccgtggccccggggcggagtggcctctcggcccatgaaccggcgcacgtcctgactgccgccggccattgtttcctaggggaagatggaggagcagccgcgtaagcaggagcaggaggaggacttaaccacccacgagcaacccaaaatcgagcaggacctgggcttcgaagagccggctcgtctagaacccccacaggatgaacaggagcacgagcaagacgcaggccaggaggagaccgacgctgggctcgagcatggctacctgggaggagaggaggatgtgctgctgaaacacctgcagcgccagtccctcatcctccgggacgccctggccgaccggagcgaaacccccctcagcgtcgaggagctgtgtcgggcctacgagctcaacctcttctcgccgcgcgtaccccccaaacgccagcccaacggcacctgcgagcccaacccgcgtctcaacttctatcccgtctttgcggtccccgaagccctcgccacctatcacatctttttcaagaaccaaaagatccccgtctcctgccgcgccaaccgcaccagcgccgacgcgctcctcgctctggggcccggcgcgcgcatacctgatatcgcttccctggaagaggtgcccaagatcttcgaagggctcggtcgggacgagacgcgcgcggcgaacgctctgaaagaaacagcagaggaagagggtcacactagcgccctggtagagttggaaggcgacaacgccaggctggccgtgctcaagcgcagcgtcgagctcacccacttcgcctaccccgccgtcaacctcccgcccaaggtcatgcgtcgcatcatggatcagctcatcatgccccacatcgaggccctcgatgaaagtcaggagcagcgccccgaggacgcccggcccgtggtcagcgacgagatgctcgcgcgctggctcgggacccacgacccccaggctttggaacagcggcgcaagctcatgctggccgtggtcctggttaccctcgagctggaatgcatgcgccgcttcttcagcgaccccgagaccctgcgcaaggtcgaggagaccctgcactacactttcagacacggtttcgtcaggcaggcctgcaagatctccaacgtggagctgaccaacctggtctcctgcctggggatcctgcacgagaaccgcctggggcagaccgtgctccactctaccctgaagggcgaggcgcggcgggactatgtccgcgactgcgtctttctatttctttgccacacatggcaagcagccatgggcgtgtggcaacagtgtctcgaggacgataacctgaaggagctggacaagcttcttgctagaaatcttaaaaagctgtggacgggcttcgacgagcgcaccgtcgcctcggacctggccgagatcgtgttccccgagcgcctgaggcagacgctgaaaggcgggctgcccgacttcatgagccagagcatgttgcaaaactaccgcactttcattctcgagcgatctgggatgctgcccgccacctgcaacgctttcccctccgactttgtcccgctgagctaccgcgagtgtcccccgccgctgtggagccactgctacctcttgcagctggccaactacatcgcctaccactcggacgtgatcgaggacgtgagcggcgaggggctgctcgagtgccactgccgctgcaacctgtgctccccgcaccgctccctggtctgcaacccccagctactaagcgagacccaggtcatcggtacctttgagctgcaaggtccgcaggagtccaccgctccgctgaaactcacgccggggttgtggacttccgcgtacctgcgcaaatttgtacccgaggactaccacgcccacgagataaagttcttcgaggaccaatcgcgtccgcagcacgcggatctcacggcctgcgtcatcacccagggcgcaatcctcgcccaattgcacgccatccaaaaatcccgccaagagtttcttctgaaaaagggtagaggggtctacctggacccccagacgggcgaagtgctcaacccgggtctcccccagcatgccgaggaagaagcaggagccgctagtggaggagatggaagaagaatgggacagccaggcagaggaggacgaatgggaggaggagacagaggaggaagaattggaagaggtggaagaggagcaggcaacagagcagcccgtcgccgcaccatccgcgccggcagccccgccggtcacggatacaacctccgcagctccggccaagcctcctcgtagatgggatcgagtgaagggtgacggtaagcacgagcggcagggctaccgatcatggagggcccacaaagccgcgatcatcgcctgcttgcaagactgcggggggaacatcgctttcgcccgccgctacctgctcttccaccgcggggtaaacatcccccgcaacgtgttgcattactaccgtcaccttcacagctaagaaaaagcaagtaaaaggagtcgccggaggaggaggaggcctgaggatcgcggcgaacgagcccttgaccaccagggagctgaggaaccggatcttccccactctttatgccatttttcagcagagtcgaggtcagcagcaagagctcaaagtaaaaaaccggtctctgcgctcgctcacccgcagttgcttgtaccacaaaaacgaagatcagctgcagcgcactctcgaagacgccgaggctctgttccacaagtactgcgcgctcactcttaaagactaaggcgcgcccacccggaaaaaaggcgggaattacctcatcgccaccatgagcaaggagattcccaccccttacatgtggagctatcagccccaaatgggcctggccgcgggcgcctcccaggactactccacccgcatgaactggctcagtgccggcccctcgatgatctcacgggtcaacggggtccgcagtcatcgaaaccagatattgttggagcaggcggcggtcacctccacgcccagggcaaagctcaacccgcgtaattggccctccaccctggtgtatcaggaaatccccgggccgactaccgtactacttccgcgtgacgcactggccgaagtccgcatgactaactcaggtgtccagctggccggcggcgcttcccggtgcccgctccgcccacaatcgggtataaaaaccctggtgatccgaggcagaggcacacagctcaacgacgagttggtgagctcttcgatcggtctgcgaccggacggagtgttccaactagccggagccgggagatcctccttcactcccaaccaggcctacctgaccttgcagagcagctcttcggagcctcgctccggaggcatcggaaccctccagtttgtggaggagtttgtgccctcggtctacttcaaccccttctcgggatcgccaggcctctacccggacgagttcataccgaacttcgacgcagtgagagaagcggtggacggctacgactgaatgtcccatggtgactcggctgagctcgctcggttgaggcatctggaccactgccgccgcctgcgctgcttcgcccgggagagctgcggactcatctactttgagtttcccgaggagcaccccaacggccctgcacacggagtgcggatcaccgtagagggcaccaccgagtctcacctggtcaggttcttcacccagcaacccttcctggtcgagcgggaccggggcgccaccacctacaccgtctactgcatctgtccaaccccgaagttgcatgagaatttttgttgtactctttgtggtgagtttaataaaagctaaactcttgcaatactctggaccttgtcgtcgtcaactcaacgagaccgtctacctcaccaaccagactgaggtaaaactcacctgcagaccacacaagacctatatcatctggttcttcgagaacacctcatttgcagtctccaacactcactgcaacgacggtgttgaacttcccaacaacctttccagtggactgagttacgatacacatagagctaagctcgtcctctacaatccttttgtagagggaacctaccagtgccagagcggaccttgtactcacaccttccatttggtgaacgtcaccagcagcagcaacagctcagaaactaaccttccttctgatactaacaaacctcgtttcggaggtgagctaaggcttcccccttctgaggagggggttagcccatacgaagtggtcgggtatttgattttaggggtggtcctgggtgggtgcatagcggtgctagctcagctgccttgctgggtggaaatcaaaatctttatatgctgggtcagatattgtggggaggaaccatgaaggggcttttgctgattatccttttcatggtggggggtgtactgtcatgccacgaacagccacgatgtaacatcaccacaggcaatcatatgagcagagagtgcactgtagtcatcaaatgcgagcacgactgcccactaaacattacattcaagaataacaccatgggaaatgtatgggtgggtttctgggaaccaggagatgagcagaactacacggtcactgtccatggtagcaatggaaatcacactttcggtttcaaattcatttttgaagtcatgtgtgatatcacactgcatgtggctagacttcatggcttgtggccccctaccaaggagaacatggttgggttttctttggcttttgtgatcatggcctgcttgatgtcaggtctgctggtaggggctttagtgtggttcctgaagcgcaagcctaggtacggaaatgaagaaaaggaaaaattgctataatctttttctttttcacagaaccatgaatgctttgaccagtgtcgtgctgctctctcttcttgtagcttttagtaatggggaagctgaaactgtagttgtaaatgttaaatctggtacaaaccacacccttgaaggtcctagaaaaactccagttcagtggtatgggggtgctaactttgacatgttttgcaatggctctaaaatacatcacaatgaattgaatcacacttgctctattcagaacataactcttacattcataaacagaacacatcatggaacatactatggttttggctctgacaatcaaaattcaaaagtgtatcatgtcagagtagatgtagagcctcctagaccccgtgctactttggctcctcctcaggacataactattaagtatggctcaaatagaacattgcagggcccaagtgttactccagttagttggtatgatggtgaaggaaatcggttttgcgatggcgataaaattgatcatacagaaattaatcacacttgcaatgctcaaaaccttactttgctgtttgtgaatgaaacacatgaaagaacatattatggaattagtggtgattggaaacagcgaaatgagtatgatgttactgttacaaagacacaattaaatattaaaaatttgggccaacgcaaaactgatgaaaaccataaaaatggaatgcatcagaaagtcgaacaaaatcctgaaactaagaaagaacagaagccttcaaaaagacctagacaaaaaacattgcaaactacaattcaggttatgattcctattggaactaattatactttagtggggccttcgccaccagtgagctggcatactacaaaaaatggcttaacagaactctgtaatggaaaccctattttaagacacacttgtgatgggcaaaatattacacttattaatgttaatgctacatttgaggctgattactatggctcgaacaataagagtgaatcaaaacactacagagtcaaggttttcaaagaaagaaaagatcaggcactattattcagaccgcttactaccaaaggaagcatgatcattactactgaaaatcaaaactttgaattacaacaaggtgacaatcaagatgatgacaaaattccatcaactactgtggcaatcgtggtgggtgtgattgcgggctttgtgactctgatcattgtcttcatatgctacatctgctgccgcaagcgtcccaggtcatacaatcatatggtagacccactactcagcttctcttactgaaactcagtcactctcatttcagaaccatgaaggctttcacagcttgcgttctgattagcatagtcacacttagtgcagctgaagctaaatgctttcatacttataacttaactagaggggaaaatattacattagcaggtgctggcttaaacacaacatgggaagcatatcacaatggatggaaacaagtttgtccatggaatgacggtcgctatgtgtgcgttggaaacagcagtaccataactaatcttacagttgtagctaatgcaaatttatcatcaactgttaaatttagagctgaaagtttatacattggaacagatggatatgaaagcaatccatcatgcttttatactatcaatgtaattgagcttccaaccaccagatcgccaactaccaccacggtcagtacaactactgagaccacaactcacactacacagttagacactacagtgcagaatagtactgtattggttaggtatttgttaagggaggaaagtactactgaacagacagaggctacctcaagcgccttcagcagcacttcaaatttaacttcgcttgcttggactaatgaaaccggagtatcattgatgcatggccagccttactcaggtttggatattcaaattacttttctggttgtctgtgggatctttattcttgtggttcttctgtactttgtctgctgcaaagccagagaaaaatctaggcggcccatctacaggccagtaatcggggaacctcagccactccaagtggatggaggcttaaggaatcttcttttctcttttacagtatggtgatcagccatgattcctaggttcttcctatttaacatcctcttctgtctcttcaacgtgtgcgctgccttcgcggccgtctcgcacgcctcacccgactgtctcgggcccttccccacctacctcctctttgccctgctcacctgcacctgcgtctgcagcattgtctgcctggtcatcaccttcctgcagctcatcgactggtgctgcgcgcgctacaattacctgcatcatagtcccgaatacagggacgagaacgtagccagaatcttaaggctcatatgaccatgcagactctgctcatactgctatccctcctatcccctaccctcgccacttctgctgattactctaaatgcaaattcgcggacatatggaatttcttagactgctatcaggagaaaattgacatgccctcctattacttggtgattgtgggaatagttatggtctgctcctgcactttctttgccatcatgatctacccctgttttgatctcggctggaactctgttgaagcattcacatacacactagaaagcagttcactagcctccacgccaccacccacaccgcctccccgcagaaatcagtttcccatgattcagtacttagaagagccccctccccgacccccttccactgttagctactttcacataaccggcggcgatgactgaccaccacctggacctcgagatggacggccaggcctccgagcagcgcatcctgcaactgcgcgtccgtcagcagcaggagcgtgccgccaaggagctcctcgatgccatcaacatccaccagtgcaagaagggcatcttctgcctggtcaaacaggcaaagatcacctacgagctcgtgtccggcggcaagcagcatcgcctcgcctatgagctgccccagcagaagcagaagttcacctgcatggtgggcgtcaaccccatagtcatcacccagcagtcgggcgagaccagcggctgcatccactgctcctgcgaaagccccgagtgcatctactccctgctcaagaccctttgcggactccgcgacctcctccccatgaactgatgttgattaaaagcccaaaaaccaatcagccccttcccccatttccccatcccccaattactcataaaaaataaatcattggaattaatcattcaataaagatcacttacttgaaatctgaaagtatgtctctggtgtagttgttcagcagcacctcggtaccctcctcccagctctggtactccagtccccggcgggcggcgaacttcctccacaccttgaaagggatgtcaaattcctggtccacaattttcattgtcttccctctcagatggcaaagaggctccgggtggaagatgacttcaaccccgtctacccctatggctacgcgcggaatcagaatatccccttcctcactcccccctttgtctcctccgatggattcaaaaacttcccccctggggtcctgtcacttaaactggctgatccaatcaccatcaacaatggggatgtctcacttaaggtgggagggggacttgctgtagagcaacagactggtaacctaagcgtaaaccctgatgcacccttgcaagttgcaagtgataagctacagcttgctctggctcctccattcgaggtcagagatggaaagcttgctttaaaggcaggtaatggattaaaagtactagataattccattactggattgactggattattgaatacacttgtggtattaactggaaggggaataggaacggaggaattaaaaaatgacgatggtgtaacaaacaaaggagtcggcttgcgtgtaagacttggagatgacggcgggctgacatttgataaaaagggtgatttagtagcctggaataaaaaagatgacaggcgcaccctgtggacaacccctgacacatctccaaattgcaaaatgagtacagaaaaggattctaaacttacgttgacacttacaaagtgtggaagtcaggttctgggaaatgtatctttacttgcagttacaggtgaatatcatcaaatgactgctactacaaagaaggatgtaaaaatatctttactatttgatgagaatggaattctattaccatcttcgtcccttagcaaagattattggaattacagaagtgatgattctattgtatctcaaaaatataataatgcagttccattcatgccaaacctgacagcttatccaaaaccaagcgctcaaaatgcaaaaaactattcaagaactaaaatcataagtaatgtctacttaggtgctcttacctaccaacctgtaattatcactattgcatttaatcaggaaactgaaaatggatgtgcttattctataacatttaccttcacttggcaaaaagactattctgcccaacagtttgatgttacatcttttaccttctcatatcttacccaagagaacaaagacaaagactaataaaatgttttgaactgaatttatgaatctttatttatttttacaccagcacgggtagtcagtttcccaccaccagcccatttcacagtgtaaacaattctctcagcacgggtggccttaaatagggaaatgttctgattagtgcgggaactggacttggggtctataatccacacagtttcctggcgagccaaacgggggtcggtgattgagatgaagccgtcctctgaaaagtcatccaagcgggcctcacagtccaaggtcacagtctggtggaatgagaagaacgcacagattcatactcggaaaacaggatgggtctgtgcctctccatcagcgccctcaacagtctttgccgccggggctcggtgcggctgctgcagatgggatcgggatcgcaagtctctctgactatgatccccacagccttcagcaacagtctcctggtgcgtcgggcacagcaccgcatcctgatctctgccatgttctcacagtaagtgcagcacataatcaccatgttattcagcagcccataattcagggcgctccaaccaaagctcatgttggggatgatggaacccacgtgaccatcgtaccagatgcggcagtatatcaggtgcctgcccctcatgaacacactgcccatatacatgatctctttgggcatgtttctgttcacaatctgccggtaccatgggaatcgctggttgaacatgcacccgtaaatgactctcctgaaccacacggccagcatggtgcctcccgcccgacactgcagggatcccggggctgaacagtggcaatgcaggatccagcgctcgtacccgctcaccatctgagctctcaccaagtccagggtagcggggcacaggcacactgacatacatctttttaaaatttttatttcctctggggtcaggatcatatcccaggggactggaaactcttggagcagggtaaagccagcagcacatggtaatccacggacagaacttacattatgataatctgcatgatcacaatcgggcaacagggggtgttgttcagttagtgaggccctagtctcctcctcacatcgtggtaaacgggccctgcggtaaggatgatggcggagcgagctcgactgttcctcggtggacattgaaatggattctcttgcgtaccttgtcgtacttctgccagcagaaagtggctcgggaacagcagatacctttcctcctgctgtccttccgctgctgacgctcagtcatccaactgaagtacagccattcccgcaggttctccagcagctcctgtgcatctgatgaaacaaaagtcccgtcgatgcggattccccttaaaacatcagccaggacattgtaggccatcccaatccagttaatgcatcctgatctatcatgaagaggaggtgggggaagaactggaagaaccatttttattccaagcggtctcgaaggacgataaagtgcaagtcacgcaggtgacagcgttccccgccgctgtgctggtggaaacagacagccaggtcaaaacccactctattttcaaggtgctcgactgtggcttcgagcagtggctctacgcgtacatccagcataagaatcacattaaaggctggacctccatcgatttcatcaatcatcaggttacactcattcaccatccccaggtaattctcatttttccagccttggattatttctacaaattgttggtgtaagtccactccgcacatgtggaaaagttcccacagcgccccctccactttcataatcaggcagaccttcatattagaaacagatcctgctgctccaccacctgcagcgtgttcaaaacaacaagattcaatgaggttctgccctctgccctcagctcacgtctcagcgtcagctgcaaaaagtcactcaagtcctcagccactacagctgacaattcagagccagggctaagcgtgggactggcaagcgtgagtgagtactttaatgctccaaagctagcacccaaaaactgcatgctggaataagctctctttgtgtcaccggtgatgccttccaataggtgagtgataaagcgaggtagtttttctttaatcatttgagtaatagaaaagtcctctaaataagtcactaggaccccaggaaccacaatgtggtagctgacagcgtgtcgctcaagcatggttagtagagatgagagtctgaaaaacagaaagcatgcactaaaccagagttgccagtctcactgaaggaaaaatcactctctccagcagcaaagtgcccactgggtggccctctcggacatacaaaaatcgatccgtgtggttaaagagcagcacagttagctcctgtcttctcccagcaaagatcacatcggactgggttagtatgcccctggaatggtagtcattcaaggccataaatctgccttggtagccattaggaatcagcacgctcactctcaagtgaaccaaaaccaccccatgcggaggaatgtggaaagattctgggcaaaaaaaggtatatctattgctagtcccttcctggacgggagcaatccctccagggctatctatgaaagcatacagagattcagccatagctcagcccgcttaccagtagacagagagcacagcagtacaagcgccaacagcagcgactgactacccactgacccagctccctatttaaaggcaccttacactgacgtaatgaccaaaggtctaaaaaccccgccaaaaaaacacacacgccctgggtgtttttcgcgaaaacacttccgcgttctcacttcctcgtatcgatttcgtgactcaacttccgggttcccacgttacgtcacttctgcccttacatgtaactcagccgtagggcgccatcttgcccacgtccaaaatggcttccatgtccggccacgcctccgcggcgaccgttagccgtgcgtcgtgacgtcatttgcatcaccgtttctcgtccaatcagcgttggctccgccccaaaaccgttaaaattcaaaagctcatttgcatattaacttttgtttactttgtggggtatattattgatgatg

SEQ ID NO: 2 - DNA, 35206 bp, Human Adenovirus serotype 48
catcatcaataatataccccacaaagtaaacaaaagttaatatgcaaatgagcttttgaatttagggcgtggccgacgctgattggacaagagaagatgacgcaaatgacgtcacgacgcacgggtcaacggtcgccgcggaggcgtggcctagcccggaagcaagtcgcggggctgatgacgtataaaaaagcggactttagacccggaaacggccgattttcccgcggccacgcccggatatgaggtaattctaggcggatgcaagtgaaattaggtcattttggcgcgaaaactgaatgaggaagtgaaaagcgaaaaataccggtcccgcctagggcggaatatttaccgagggccgagagactttgaccgattacgtgggggtttcgattgcggtgtttttttcgcgaatttccgcgtccgtgtcaaagtccggtgtttatgtcacagatcagctgatccacagggtatttaaaccagtcgagcccgtcaagaggccactcttgagtgccagcgagtagagatttctctgagctccgctcccagagtctgagaaaaatgagacacctgcgcctcctgcctggaactgtgcctatggacatggctatgcttctactagatgactttgtgaatacagtattggaggacgaactgcaaccaactccgttcgagctgggacccacacttcaggacctctatgatctggaggttgatgcctatgaggacgacccgaacgaagaggctgtaaatttaatatttccagaatctatgattcttcaggctgacatagccagcgaatctatacctactccacttcatacaccgactctaccacccatacctgaattggaagaggaggacgaaatagacctccggtgttatgaggaaggttttcctcccagcgattcagaggacgaacgtggtgagcagagtatgtctataatctcagactatgcttgtgtggttgtggaagagcattttgtgttggacaatcctgaggtgccagggcaaggctgtagatcctgtcaatatcaccgggatcagaccggagaccaaaatgcttcctgcgctctgtgttacatgaaaatgagcttcagctttatttacagtaagtggagtgaatgtgagagaggctgagtgtttaacacatcactgtgtatggcttgcagctgtgctaagtgtggtttatttttgttactaggtccggtgtcagaggatgagtcatcaccctcagaagaagaccacccgtctccccctgagctgtcaggcgaaacgcccctgcaagtgcacagacccaccccagtcagagccagtggcgagaggcgagcagctgtagaaaaaattgaggacttgttacatgacatgggtggggatgaacctttggacctgagcttgaaacgccccaggaactaggcgcagctgcgcttagtcatgtgtaaataaagttgtacaataaaagtatatgtgacgcatgcaaggtgtggtttatgactcatgggcggggcttagtcctatataagtggcaacacctgggcattcaggcacagaccttcagggagttcctgatggatgtgtggactatccttgcagactttagcaagacacgccggcttgtagaggatagttcagacgggtgctccgggttctggagacactggtttggaactcctctatctcgcctggtgtacacagttaagaaggattataaagaggaatttgaaaatctttttgctgactgctctggcctgctagattctctgaatcttggccaccagtcccttttccaggaaagggtactccacagccttgatttttccagcccagggcgcactacagccggggttgcttttgtggtttttctggttgacaaatggagccaggacacccaactgagcaggggctacatcctggacttcgcagccatgcacctgtggagggcctggatcaggcagcggggacagagaatcttgaactactggcttctacagccagcagctccgggtcttcttcgtctacacagacaaacatccatgttggaggaagaaatgaggcaggccatggacgagaacccgaggagcggcctggaccctccgtcggaagaggagctggattgaatcaggtatccagcctgtacccagagcttagcaaggtgctgacatccatggccaggggagtgaagagggagaggagcgatgggggcaataccgggatgatgaccgagctgactgccagtctgatgaatcgcaagcgcccagagcgccttacctggtacgagctacagcaggagtgcagggatgagataggcctgatgcaggataaatatggcctggagcagataaaaacccactggttgaacccagatgaggattgggaggaggccattaagaaatatgccaagatagccctgcgcccagattgcaagtacatagtgaccaagaccgtgaatatcagacatgcctgctacatctcggggaacggggcagaggtgatcattgataccctggacaaggccgccttcaggtgttgcatgatgggaatgagagccggagtgatgaatatgaattccatgatcttcatgaacatgaagttcaatggagagaagtttaatggggtgctgttcatggccaacagccacatgaccctgcatggctgcagtttctttggcttcaacaatatgtgtgcagaggtctggggcgctgctaagatcaggggatgtaagttttatggctgctggatgggcgtggtcggaagacccaagagcgagatgtctgtgaagcagtgtgtgtttgagaaatgctacctgggagtctctaccgagggcaatgctagagtgagacactgctcttccctggagacgggctgcttctgcctggtgaagggtacagcctctctgaagcataatatggtgaagggctgcacggatgagcgcatgtacaacatgctgacctgcgattcgggggtctgccatatcctgaagaacatccatgtgacctcccaccccagaaagaagtggccagtgtttgagaataacctgctgatcaagtgccatatgcacctgggcgccagaaggggcaccttccagccgtaccagtgcaactttagccagaccaagctgctgttggagaacgatgccttctccagggtgaacctgaacggcatctttgacatggatgtctcggtgtacaagatcctgagatacgatgagaccaaatccagggtgcgtgcttgcgagtgcgggggcagacacaccaggatgcaaccagtggccctggatgtgaccgaggagctgagacccgaccacctggtgatggcctgtaccgggaccgagttcagctccagtggggaggacacagattagaggtaggttttgagtagtgggcgtggctaaggtgactataaaggcgggtgtcttacgagggtctttttgcttttctgcagacatcatgaacgggaccggcggggccttcgaaggggggctttttagcccttatttgacaacccgcctgccgggatgggccggagttcgtcagaatgtgatgggatcgacggtggatgggcgcccagtgcttccagcaaattcctcgaccatgacctacgcgaccgtggggagctcgtcgctcgacagcaccgccgcagccgcggcagccgcagccgccatgacagcgacgagactggcctcgagctacatgcccagcagcggtagcagcccctctgtgcccagttccatcatcgccgaggagaaactgctggccctgctggccgagctggaagccctgagccgccagctggccgtcctgacccagcaggtgtccgagctccgcgagcagcaacagcagcaaaataaatgattcaataaacacagattctgattcaaacagcaaagcatctttattatttattttttcgcgcgcggtaggccctggtccacctctcccgatcattgagagtgcggtggattttttccaggacccggtagaggtgggattggatgttgaggtacatgggcatgagcccgtcccgggggtggaggtagcaccactgcatggcctcgtgctctggggtcgtgttgtagatgatccagtcatagcatgggcgctgggcgtggtgctggatgatgtccttgaggaggagactgatggccacggggagccccttggtgtaggtgttggcgaagcggtttagctgggagggatgcatgcggggggagatgatgtgcagtttggcctggatcttgaggttggcaatgttgccgcccagatcccgcctggggttcatgttgtgcaggaccaccaggacggtgtagcccgtgcacttggggaacttgtcatgcaacttggaagggaaagcgtggaagaatttggagacgcccttgtgcccgcccaggttttccatgcactcatccatgatgatggcgatgggcccgtgggctgcggctttggcaaagacgtttctggggtcagagacatcataattatgctcctgggtgagatcatcataagacattttaatgaatttggggcggagggtgccagattgggggacgatggttccctcgggccccggggcaaagttcccctcgcagatctgcatctcccaggctttcatctcggagggggggatcatgtccacctgcggggcgatgaaaaaaacggtttccggggcgggggtgatgagctgcgaggagagcaggtttctcaacagctgggacttgccgcacccggtcgggccgtagatgaccccgatgacgggttgcaggtggtagttcaaggacatgcagctgccgtcgtcccggaggaggggggccacctcgttgagcatgtctctgacttggaggttttcccggacgagctcgccaaggaggcggtccccgcccagcgagagcagctcttgcagggaagcaaagtttttcaggggcttgagcccgtcagccatgggcatcttggcgagggtctgcgagaggagctccaggcggtcccagagctcggtgacgtgctctacggcatctcgatccagcagacttcctcgtttcgggggttgggacgactgcgactgtagggcacgagacgatgggcgtccagcgcggccagcgtcatgtccttccagggtctcagggtccgcgtgagggtggtctccgtcacggtgaaggggtgggccccgggctgggcgcttgcaagggtgcgcttgagactcatcctgctggtgctgaaacgggcacggtcttcgccctgcgcgtcggcgagatagcagttgaccatgagctcgtagttgagggcctcggcggcgtggcccttggcgcggagcttgcccttggaagagcgcccgcaggcgggacagaggagggattgcagggcgtagagcttgggcgcgagaaagaccgactcgggggcgaaagcatccgctccgcagtgggcgcagacggtctcgcactcgacgagccaggtgagctcgggctgctcggggtcaaaaaccagttttcccccgttctttttgatgcgcttcttacctcgcgtctccatgagtctgtgtccgcgctcggtgacaaacaggctgtcggtgtccccgtagacggacttgattggcctgtcctgcaggggcgtcccgcggtcctcctcgtagagaaactcggaccactctgagacgaaggcgcgcgtccacgccaagacaaaggaggccacgtgcgaggggtagcggtcgttgtccaccagggggtccaccttttccaccgtgtgcagacacatgtccccctcctccgcatccaagaaggtgattggcttgtaagtgtaggccacgtgaccgggggtccccgacgggggggtataaaagggggcgggtctgtgctcgtcctcactctcttccgcgtcgctgtccacgagcgccagctgttggggtaggtattccctctcgagagcgggcatgacctcggcactcaggttgtcagtttctagaaatgaggaggatttgatgttggcttgccctgccgcgatgctttttaggagactttcatccatctggtcagaaaagactatttttttattgtcaagcttggtggcgaaggagccatagagggcgttggatagaagcttggcgatggatctcatggtctgatttttgtcacggtcggcgcgctccttggccgcgatgttgagctgaacatattcgcgcgcgacacacttccattcgggaaagacggtggtgcgctcgtcgggcacgatcctgacgcgccagccgcggttatgcagggtgaccaggtccacactggtggccacctcgccgcgcaggggctcgttggtccagcagagtctgccgcccttgcgcgagcagaaagggggcagcacatccaggaggtgctcgtcaggggggtccgcatcgatggtgaagatgccgggacagagttccttgtcaaaatagtctatttttgaggatgcatcatccaaggccatctgccactcgcgggcagccatcgctcgctcgtaggggttgaggggcggaccccatggcatggggtgcgtgagcgcggaggcatacatgccgcagatgtcatagacatagatgggctccgagaggatgccgatgtaggtgggataacagcgccccccgcggatgctggcgcgcacgtagtcatacaactcgtgcgagggggccaagaaggcggggccgagattggtgcgctggggctgctcggcgcggaagacgatctggcgaaagatggcatgcgagttggaggagatggtgggccgttggaagatgttaaagtgggcgtggggcaggcggaccgagtcgcggatgaagtgcgcgtaggagtcttgcagcttggcgacgagctcggcggtgacgaggacgtccatggcgcagtagtccagcgtttcgcggatgatgtcataacccgcctctcctttcttctcccacagctcgcggttgagggcatactcctcgtcatccttccagtactcccggagcgggaatcctcgatcgtccgcacggtaagagcccagcatgtagaaatggttcacggccttgtagggacagcagcccttctccacggggagggcgtaagcttgagcggccttgcggagcgaggtgtgcgtcagggcgaaggtgtccctgaccatgactttcaagaactggtacttgaagtccgagtcgtcgcagccgccgtgctcccagagctcgaaatcggtgcgcttcttcgagagggggttaggcagagcgaaagtgacgtcattgaagagaatcttgcctgcccgtggcataaaattgcgggtgatgcggaaagggccaggcacggaggctcggttgttgatgacctgggcggcgaggacgatctcgtcgaagccgttgatgttgtgcccgacgatgtagagttccatgaatcgcgggcggcctttgatgtgcggcagctttttgagctcctcgtaggtgaggtcctcggggcattgcaggccgtgctgctcgagcgcccactcctggagatgtgggttggcttgcatgaaggaagcccagagctcgcgggccatgagggtctggagctcgtcgcgaaagaggcggaactgctggcccacggccatcttttctggggtgacgcagtagaaggtgagggggtcccgctcccagcgatcccagcgtaagcgcacggcgagatcgcgagcgagggcgaccagctcggggttcccggagaatttcatgaccagcatgaaggggacgagctgcttgccgaaggaccccatccaggtgtaggtttctacatcgtaggtgacaaagagccgctccgtgcgaggatgagagccgattgggaagaactggatttcctgccaccagttggacgagtggctgttgatgtgatgaaagtagaaatcccgccggcgaaccgagcactcgtgctgatgcttgtaaaagcgtccgcagtactcgcagcgctgcacgggctgtacctcatccacgagatacacagcgcgtcccttgaggaggaacttcaggagtggcggccctggctggtggttttcatgttcgcctgcgtgggactcaccctggggctcctcgaggacggagaggctgacgagcccgcgcgggagccaggtccagatctcggcgcggcgggggcggagagcgaagacgagggcgcgcagttgggagctgtccatggtgtcgcggagatccaggtccgggggcagggttctgaggttgacctcgtagaggcgtgtgagggcgtgcttgagatgcagatggtacttgatttctacgggtgagttggtggccgtgtccacgcattgcatgagcccgtagctgcgcggggccacgaccgtgccgcggtgtgcttttagaagcggtgtcgcggacgcgctcccggcggcagcggcggttccggccccgcgggcaggggcggcagaggcacgtcggcgtggcgctcgggcaggtcccggtgctgcgccctgagagcgctggcgtgcgcgacgacgcggcggttgacatcctggatctgccgcctctgcgtgaagaccactggccccgtgactttgaacctgaaagacagttcaacagaatcaatctcggcgtcattgacggcggcctgacgcaggatctcttgcacgtcgcccgagttgtcctggtaggcgatctcggacatgaactgctcgatctcctcctcctggagatcgccgcggcccgcgcgctcgacggtggcggcgaggtcatttgagatgcgacccatgagctgcgagaaggcgcccaggccgctctcgttccagacgcggctgtagaccacgtccccgtcggcgtcgcgcgcgcgcatgaccacctgcgcgaggttgagctccacgtgccgcgtgaagacggcgtagttgcgcaggcgctggaagaggtagttgagggtggtggcgatgtgctcggtgacgaagaagtacatgatccagcggcgcagaggcatctcgctgatgtcgccgatgccctccagcctttccatggcctcgtagaaatccacggcgaagttgaaaaactgggcattgcgggccgagaccgtgagctcgtcttccaggagccggatgagctcggcgatggtggcgcgcacctcgcgctcgaaatccccaggggcctcctcctcttcctcttcttccatgacgacctcttcttctatttcttcctctgggggcggtggtggtggcggggcccgacgacgacggcgacgcaccgggagacggtcgacgaagcgctcgatcatctccccgcggcggcgacgcatggtttcggtgacggcgcgaccccgttcgcgaggacgcagcgtgaagacgccgccggtcatctcccggtaatggggcgggtccccgttgggcagcgatagggcgctgacgatgcatcttatcaattgcggtgtaggggacgtgagcgcgtcgagatcgaccggatcggagaatctttcgaggaaagcgtctagccaatcgcagtcgcaaggtaagctcaaacacgtagcagccctgtggacgctgttagaattgcggttgctgatgatgtaattgaagtaggcgtttttgaggcggcggatggtggcgaggaggaccaggtccttgggtcccgcttgctggatgcggagccgctcggccatgccccaggcctggccctgacaccggctcaggttcttgtagtagtcatgcatgagcctctcaatgtcatcactggcggaggcggagtcttccatgcgggtgaccccgacgcccctgagcggctgcacgagcgccaggtcggcgacgacgcgctcggcgaggatggcctgttgcacgcgggtgagggtgtcctggaagtcgtccatgtcgacgaagcggtggtaggccccggtgttgatggtgtaggtgcagttggccatgatcgaccagttgacggtctgcaggccgggctgcacgacctcggagtacctgagccgcgagaaggcgcgcgagtcgaagacgtagtcgttgcaggtgcgcacgaggtactggtagccgactaggaagtgcggcggcggctggcggtagagcggccagcgctgggtggccggcgcgcccggggccaggtcctcgagcatgaggcggtggtagccgtagaggtagcgggacatccaggcgatgccggcggcggtggtggaggcgcgcgggaactcgcggacgcggttccagatgttgcgcagcggcaggaaatagtccatggtcggcacggtctggccggtgagacgcgcgcagtcattgacgctctagaggcaaaaacgaaagcggttgagcgggctcttcctccgtagcctggcggaacgcaaacgggttaggtcgcgtgtgtaccccggttcgagtcccctcgaatcaggctggagccgcgactaacgtggtattggcactcccgtctcgacccgagcccgatagccgccaggatacggcggagagccctttttgccggccgaggggggtcgctagacttgaaagcgaccgaaaaccctgccgggtagtggctcgcgcccgtagtctggagaagcatcgccagggttgagtcgcggcagaacccggttcgaggacggccgcggcgagcgggacttggtcaccccgccgatttaaagacccacagccagccgacttctccagttacgggagcgagcccccttttttctttttgccagatgcatcccgtcctgcgccaaatgcgtcccacccccccggcgaccaccgcgaccgcggccgtagcaggcgccggcgctagccagccacagacagagatggacttggaagagggcgaagggctggcgagactgggggcgccgtccccggagcgacacccccgcgtgcagctgcagaaggacgtgcgcccggcgtacgtgcctgcgcagaacctgttcagggaccgcagcggggaggagcccgaggagatgcgcgactgccgatttcgggcgggcagggagctgcgcgagggcctggaccgccagcgcgtgctgcgcgacgaggatttcgagccgaacgagcagacggggatcagccccgcgcgcgcgcacgtggcggcggccaacctggtgacggcctacgagcagacggtgaagcaggagcgcaacttccaaaagagtttcaacaaccacgtgcgcaccctgatcgcgcgcgaggaggtggccctgggcctgatgcacctgtgggacctggcggaggccatcgtgcagaacccggacagcaagcctctgacggcgcagctgttcctggtggtgcagcacagtagggacaacgaggcgttcagggaggcgctgctgaacatcgccgagcccgagggccgctggctgctggagctgatcaacatcttgcagagcatcgtagtgcaggagcgcagcctgagcctggccgagaaggtggcggccatcaactactcggtgctgagcctgggcaagttttacgcgcgcaagatttacaagacgccgtacgtgcccatagacaaggaggtgaagatagacagcttttacatgcgcatggcgctcaaggtgctgacgctgagcgacgacctgggcgtgtaccgcaacgaccgcatccacaaggccgtgagcacgagccggcggcgcgagctgagcgaccgcgagctgatgctgagcctgcgccgggcgctggtagggggcgccgccggcggcgaggagtcctacttcgacatgggggcggacctgcattggcagccgagccggcgcgccttggaggccgcctacggtccagaggacttggatgaggatgaggaagaggaggaggatgcacccgctgcggggtactgacgcctccgtgatgtgtttttagatgtcccagcaagccccggaccccgccataagggcggcgctgcaaagccagccgtccggtctagcatcggacgactgggaggccgcgatgcaacgcatcatggccctgacgacccgcaaccccgagtcctttagacaacagccgcaggccaacagactctcggccattctggaggcggtggtcccctctcggaccaaccccacgcacgagaaggtgctggcgatcgtgaacgcgctggcggagaacaaggccatccgtcccgacgaggccgggctggtgtacaacgccctgctggagcgcgtgggccgctacaacagcacgaacgtgcagtccaacctggaccggctggtgacggacgtgcgcgaggccgtggcgcagcgcgagcggttcaagaacgagggcctgggctcgctggtggcgctgaacgccttcctggcgacgcagccggcgaacgtgccgcgcgggcaggacgattacaccaactttatcagcgcgctgcggctgatggtgaccgaggtgccccagagcgaggtgtaccagtctggcccggactactttttccagacgagccggcagggcttgcagacggtgaacctgagccaggctttcaagaacctgcgcgggctgtggggcgtgcaggcgcccgtgggcgaccggtcgacggtgagcagcttgctgacgcccaactcgcggctgctgctgctgctgatcgcgcccttcaccgacagcggcagcgtgaaccgcaactcgtacctgggccacctgctgacgctgtaccgcgaggccataggccaggcgcaggtggacgagcagaccttccaggagatcacgagcgtgagccgcgcgctggggcagaacgacaccgacagtctgagggccaccctgaacttcttgctgaccaatagacagcagaagatcccggcgcagtacgcactgtcggccgaggaggaaaggatcctgagatatgtgcagcagagcgtagggctgttcctgatgcaggagggcgccacccccagcgccgcgctggacatgaccgcgcgcaacatggaacctagcatgtacgccgccaaccggccgttcatcaataagctgatggactacctgcaccgcgcggcggccatgaacacggactactttactaatgctatactaaacccgcactggctcccgccgccggggttttacacgggcgagtacgacatgcccgaccccaacgacgggttcctgtgggacgacgtggacagcgcggtgttctccccgaccttgcaaaagcgccaggaggcggtgcgcacgcccgcgagcgagggcgcggtgggtcgcagcccctttcctagcttagggagtttgcatagcttgccgggctcggtgaacagcggcagggtgagccggccgcgcttgttgggcgaggacgagtacctgaacgactcgctgctgcagccgccacgggtcaagaacgccatggccaataacgggatagagagtctggtggacaaactgaaccgttggaagacctacgctcaggaccatagggacgcgcccgcgccgcggcgacagcgccacgaccggcagcggggcctggtgtgggacgacgaggactcggccgacgatagcagcgtgttggacttgggcgggagcggtggggccaacccgttcgcgcatctgcagcccagactggggaggcggatgttttgaaatgcaaaataaaactcaccaaggccatagcgtgcgttctcttccttgttagagatgaggcgcgcggtggtgtctcctcctccctcgtacgagagcgtgatggcgcaggcgaccctggaggttccgtttgtgcctccgcggtatatggctcctacggagggcagaaacagcattcgttactcggagctggctccgcagtacgacaccactcgcgtgtacttggtggacaacaagtcggcggacatcgcttccctgaactaccaaaacgaccacagcaacttcctgaccacggtggtgcagaacaacgattttacccccgccgaggccagcacgcagacgataaattttgacgagcggtcgcggtggggcggtgatctgaagaccattctgcacaccaacatgcccaatgtgaacgagtacatgttcaccagcaagtttaaggcgcgggtgatggtgtctaggaagcggccagagggggcgacagatgcaagtcaggatatcttaaagtatgagtggtttgagtttacccttcccgagggcaacttttccgagaccatgaccatagacctgatgaacaacgccatcttggaaaactacttgcaagtggggcggcagaatggcgtgctggagagcgatatcggagtcaagtttgacagcaggaatttcaagctgggctgggacccggtgaccaagctggtgatgccaggggtctacacctacgaggccttccacccggacgtggtgctgctgccgggctgcggggtggacttcaccgagagccgcctgagcaacctcctgggcattcgcaagaagcaacctttccaagagggcttcaggatcatgtatgaggatctcgagggtggtaacatccccgccctcctggatgtcaagcaatatttggatagtaaaaagaagcttgaggaggcaacacagaatgcaaccagggctgctggagatatcagaggagacagtcatattccaagagctgtggaacaagcggctgaaaaggatctggtcattgtgccagtaacacaagatgaaagcaagagaagctataatgtcatagatggcacccatgacaccctgtaccgaagttggtacctgtcctatacctacggggaccccgagaagggggtgcagtcgtggacgctgctcaccaccccggacgtcacctgcggcgcggagcaagtctactggtcactgccggacctcatgcaagaccccgtcaccttccgctccacccagcaagtcagcaactaccccgtggtcggcgccgagctcatgcccttccgcgccaagagcttttacaacgacctcgccgtctactcccagctcatccgcagctacacctccctcacccacgtcttcaaccgcttccccgacaaccagatcctctgccgcccgcccgcgcccaccatcaccaccgttagtgaaaacgtgcctgctctcacagatcacgggacgctaccgctgcgcagcagtatccgcggagtccagcgagtgaccgtcactgacgcccgtcgccgcacctgtccctacgtctacaaggccctgggcatagtcgcgccgcgcgtgctttccagtcgcaccttctaaaaaatgtctattctcatctcgcccagcaataacaccggctggggtattactaggcccagcaccatgtacggaggagccaagaagcgctcccagcagcaccccgtccgcgtccgcggccacttccgcgctccctggggcgcttacaagcgcgggcggacttccaccgccgtgcgcaccaccgttgacgacgtcatcgactcggtggtcgccgacgcgcgcaactacacccccgccccctccaccgtggacgcggtcatcgacagcgtggtggccgacgcgcgcgactatgccagacgcaagagccggcggcgacggattgccaggcgccaccggagcacgcccgccatgcgcgccgctcgggctctgctgcgccgcgccagacgcacgggccgccgggccatgatgcgagccgcgcgccgcgctgccgctgcacccacccccgcaggcaggactcgcagacgagcggccgccgccgccgccgcggccatttctagcatgaccagacccaggcgcggaaacgtgtactgggtgcgcgactccgtcacgggcgtgcgcgtgcccgtgcgcacccgtcctcctcgtccctgatctaatgcttgtgtcctcccccgcaagcgacgatgtcaaagcgcaaaatcaaggaggagatgctccaggtcgtcgccccggagatttacggaccaccccaggcggaccagaaaccccgcaaaatcaagcgggttaaaaaaaaggatgaggtggacgagggggcagtagagtttgtgcgcgagttcgctccgcggcggcgcgtaaattggaaggggcgcagggtgcagcgcgtgttgcggcccggcacggcggtggtgttcacgcccggcgagcggtcctcggtcaggagcaagcgtagctatgacgaggtgtacggcgacgacgacatcctggaccaggcggcggagcgggcgggcgagttcgcctacgggaagcggtcgcgcgaagaggagctgatctcgctgccgctggacgaaagcaaccccacgccgagcctgaagcccgtgaccctgcagcaggtgctgccccaggcggtgctgctgccgagccgcggggtcaagcgcgagggcgagagcatgtacccgaccatgcagatcatggtgcccaagcgccggcgcgtggaggacgtgctggacaccgtgaaaatggatgtggagcccgaggtcaaggtgcgccccatcaagcaggtggcgccgggcctgggcgtgcagaccgtggacattcagatccccaccgacatggatgtcgacaaaaaaccctcgaccagcatcgaggtgcagaccgatccctggctcccagcctccaccgctaccgtctccacttctaccgccgccacggctaccgagcctcccaggaggcgaagatggggcgccgccagccggctgatgcccaactacgtgttgcatccttccatcatcccgacgcctggctaccgcggcacccggtattacgccagccgcaggcgcccagccagcaaacgccgccgccgcaccgccacccgccgccgtctggcccccgcccgcgtgcgccgcgtaaccacgcgccggggccgctcgctcgttctgcccaccgtgcgctaccaccccagcatcctttaattcgtgtgctgtgatactgttgcagagagatggctctcacttgccgcctgcgcatccccgtcccgaattaccgaggaagatcccgccgcaggagaggcatggcaggcagcggcctgaaccgccgccggcggcgggccatgcgcaggcgcctgagtggcggctttctgcccgcgctcatacccataatcgccgcggccattggcacgatcccgggcatagcttccgttgcgctgcaggcgtcgcagcgccgttgatgtgcgaataaagcctctttagactctgacacacctggtcctgtatatttttagaatggaagacatcaattttgcgtccctggctccgcggcacggcacgcggccgttcatgggcacctggaacgagatcggcaccagccagctgaacgggggcgccttcaattggagcagtgtctggagcgggcttaaaaatttcggctcgacgctccggacctatgggaacaaggcctggaatagtagcactgggcagttgttaagggaaaagctcaaagaccagaacttccagcagaaggtggtggacgggctggcctcgggcattaacggggtggtggacattgcgaaccaggccgtgcagcgcgagataaacagccgcctggacccgcggccgcccacggtggtggagatggaagatgcaactcctccgccgcccaagggcgagaagcggccgcggcccgacgcggaggagacgatcctgcaggtggacgagccaccctcgtacgaggaggccgtgaaggccggcatgcccaccacgcgcatcatcgcgccgctggccacgggtgtaatgaaacccgccacccttgacctgcctccaccacccacgcccgctccaccgaaggcagctccggttgtgcaggcccccccggtggcgaccgccgtgcgccgcgtccccgcccgccgccaggcccagaactggcagagcacgctgcacagtatcgtgggcctgggagtgaaaagtctgaagcgccgccgatgctattgagagagaggaaagaggacactaaagggagagcttaacttgtatgtgccttaccgccagagaacgcgcgaagatggccaccccctcgatgatgccgcagtgggcgtacatgcacatcgccgggcaggacgcctcggagtacctgagcccgggtctggtgcagtttgcccgcgccaccgacacgtacttcagcctgggcaacaagtttaggaaccccacggtggccccgacccacgatgtgaccacggaccggtcccagcgtctgacgctgcgcttcgtgcccgtggatcgcgaggacaccacgtactcgtacaaggcgcgcttcactctggccgtgggcgacaaccgggtgctagacatggccagcacttactttgacatccgcggcgtcctggaccgcggtcccagcttcaaaccctactcgggcacggcttacaacagtctggcccccaagggtgcccccaatcccagtcagtgggaagagaaaaagaatggaggaggaagcgatgctaatcaaatgcaaactcacacgtttggagttgctgccatgggtggcattgaaattacagctaagggtcttcaaattggcattgatgcaaccaaagaggaagataatggaaaggaaatatatgccgacaaaacattccagccagagcctcaaataggagaagaaaactggcaggatagtgataattactatggaggcagagccatcaagaaagaaaccaagatgaagccatgctatggctcatttgccagacctaccaatgaaaaaggcggccaggctaaattcaaaacacctgaaaaagaaggtgaagaacccaaagaacttgacatagatttgaatttctttgatattcccagtactggcacaggtggtaatggaacaaatgttaatttcaaaccagacatgataatgtatgcagaaaatgtgaacttggaaaccccagacactcatattgtatacaagccaggcaaggaagatgcaagttctgaatctaacctcacacaacagtccatgcccaacagacccaactacattggatttagggacaactttgtagggctcatgtactacaacagcactggcaacatgggtgtgctggctggtcaggcatctcagttgaatgctgtggtcgacttgcaagacagaaacaccgagctgtcttaccagctattgctagattctctgggtgacagaaccagatactttagtatgtggaactctgcggtggacagttacgatcccgatgtcaggatcattgagaatcacggtgtggaagatgaacttcccaactattgcttccccttggatggcgctggaactaacgcagtgtaccaaggtgtaaaagttaaaactactaacaatacagaatgggaaaaagacactgcagtatctgaacacaatcagatatgcaaaggcaacgtgtatgccatggagatcaacctccaggccaacctgtggaagagttttctgtactcgaacgtggccctgtacctgcccgactcctacaagtacacgccggccaacgtcacgctgcccaccaacaccaacacctacgagtacatgaacggccgcgtggtagccccctcgctggtggacgcttacatcaacattggcgcccgctggtcgctggaccccatggacaacgtcaacccattcaaccaccaccgcaacgcgggcctgcgctaccgttccatgcttctgggcaacggccgctacgtgcccttccacatccaagtgccccaaaagttctttgccatcaagaacctgctcctgctcccgggctcctacacctacgagtggaacttccgcaaggacgtcaacatgatcctgcagagttccctcggaaacgacctgcgcgtcgacggcgcctccgtccgcttcgacagcgtcaacctctacgccacctttttccccatggcgcacaacaccgcctccaccttggaagccatgctgcgcaacgacaccaacgaccagtccttcaacgactacctctcggccgccaacatgctctaccccatcccggccaaggccaccaacgtgcccatctccattccctcgcgcaactgggccgccttccgcggatggagtttcacccggctcaagaccaaggaaactccctccctcggctcgggtttcgacccctactttgtctactcgggatccatcccctacctcgacgggaccttctacctcaaccacaccttcaagaaggtttccatcatgttcgactcctcggtcagctggcctggcaacgaccggctgctgacgccgaacgagttcgagatcaagcgcagcgtcgacggggagggctacaacgtggcccaatgcaacatgaccaaggactggttcctcgtccagatgctctcccactacaacatcggctaccagggcttccacgtgcccgagggctacaaggaccgcatgtactccttcttccgcaacttccagcccatgagcaggcaggtggtcgatgagatcaactacaaggactacaaggccgtcaccctgcccttccagcacaacaactcgggcttcaccggctaccttgcgcccaccatgcgccaggggcagccctaccccgccaacttcccctacccgctcatcggccagacagccgtaccctccgtcacccagaaaaagttcctctgcgacagggtcatgtggcgcatccccttctccagcaacttcatgtccatgggcgcccttaccgacttgggtcagaacatgctctatgccaactcggcccacgcgctcgacatgaccttcgaggtggaccccatggatgagcccaccctcctctatcttctcttcgaagttttcgacgtggtcagagtgcaccagccgcaccgcggcgtcatcgaggccgtctacctgcgcacacccttctccgccggcaacgccaccacctaagcatgagcggttccagcgaacgagagctcgcggccatcgtgcgcgacctgggctgcgggccctactttttgggcacccacgacaagcgcttcccgggcttcctcgccggcgacaagctggcctgcgccatcgtcaacacggccggccgcgagaccggaggcgtgcactggctcgccttcggctggaacccgcgctcgcgcacctgctacatgttcgacccctttgggttctcggaccgccggctcaagcagatttacagcttcgagtacgaggccatgctgcgccgaagcgccctggcctcatcgcccgaccgctgtctcagcctcgagcagtccacccagaccgtgcaggggcccgactccgccgcctgcggacttttctgttgcatgttcttgcatgccttcgtgcactggcccgaccgacccatggacggaaaccccaccatgaacttgctgacgggggtgcccaacggcatgctacaatcgccacaggtgctacccaccctccggcgcaaccaggaggagctctaccgcttcctcgcgcgccactccccttactttcgatcccaccgcgccgccatcgaacacgccaccgcttttgataaaatgaaacaactgcgtgtatctcaataaacagcacttttattttacatgcactggagtatatgcaagttatttaaaagtcgaaggggttctcgcgctcgtcgttgtgcgccgcgctggggagggccacgttgcggtactggtacttgggaagccacttgaactcgggaatcaccagtttgggaacagcaatctcggggaaggtctcgctccacatgcgccggctcatctgcagggcacccagcatgtccgggccggagatcttgaaatcgcagttggggccggtgctctgcgcgcgcgagttgcggtacactgggttgcagcactggaacaccataagactggggtacttcacactggccagcacgctcttgtcgctgatctgatccttgtccaggtcctcggcgttgctcaggccgaacggggtcatcttgcacagctggcggcccaggaagggcacgctctgaggcttgtggttacactcgcagtgcacgggcatcagcatcattcccgcgccgcgctgcatattcgggtagagggccttgacaaaggccgtgatctgcttgaaagcttgctgggccttggccccctcgctgaaaaacaggccgcagctcttcccgctgaactggttattcccgcacccggcatcctgcacgcagcagcgcgcgtcatggctggtcagttgcaccacgcttcttccccagcggttctgggtcaccttggccttgctgggttgctccttcaacgcgcgctgcccgttctcgctggtcacatccatctccaccacgtggtccttgtggatcatcaccgtcccgtgcagacacttgagctggccttccacctcggtgcagccgtgatcccacaggacgcagccggtgcactcccagttcttgtgcgcgatcccgctgtggctgaagatgtaaccttgcaacatgcggcccatgatggtgctaaaggttttctgggtggtgaaggtcagttgcagaccgcgggcctcctcgttcatccaggtctggcacatcttttggaagatctcggtctgctcgggcatgagcttgtaagcatcgcgcaggccgctgtcgacgcggtagcgttccatcagcacattcatggcatccatgcccttctcccaggacgagaccagaggcagactcagggggttgcgcacgttcaggacaccgggggtcgcgggctcgacgatgcgttttccgtccttgccttccttcaacagaaccggcggctggctgaatcccactcccacgatcacggcatcttcctggggcatctcttcgtcggggtctaccttggtcacatgcttggtcttcctggcttgcttcttttttggagggctgtccacggggaccacgtcctcctcggaagacccggagcccacccgctgatactttcggcgcttggtgggcagaggaggtggcggcgaggggctcctctcctgctccggcggatagcgcgccgacccgtggccccggggcggagtggcctctcgctccatgaaccggcgcacgtcctgactgccgccggccattgtttcctaggggaagatggaggagcagccgcgtaagcaggagcaggaggaggacttaaccacccacgagcaacccaaaatcgagcaggacctgggcttcgaagagccggctcgtctagaacccccacaggatgaacaggagcacgagcaagacgcaggccaggaggagaccgacgctgggctcgagcatggctacctgggaggagaggaggatgtgctgctgaaacacttgcagcgccagtccctcatcctccgggacgccctggccgaccggagcgaaacccccctcagcgtcgaggagctgtgtcgggcctacgagctcaacctcttctcgccgcgcgtgccccccaaacgccagcccaacggcacatgcgagcccaacccgcgtctcaacttctatcccgtctttgcggtccccgaggccctctccacctatcacatctttttcaagaaccaaaagatccccgtctcctgccgcgccaaccgcacccgcgccgacgcgctccttgctctggggcccggcgcgcgcatacctgatatcgcttccctggaagaggtgcccaagatcttcgaagggctcggtcgggacgagacacgcgcggcgaacgctctgaaagaaacagcagaggaagagggtcacactagcgccctggtagagttggaaggcgacaacgccaggctggccgtgctcaagcgcagcgtcgagctcacccacttcgcctaccccgccgtcaacctcccgcccaaggtcatgcgtcgcatcatggatcagctcatcatgccccacatcgaggccctcgatgaaagtcaggagcagcgccccgaggacgcccgccccgtggtcagcgacgagatgctcgcgcgctggctcgggacccgcgacccccaggctttggagcagcggcgcaagctcatgctggccgtggtcctggtcaccctcgagctcgaatgcatgcgccgcttcttcagcgaccccgagaccctgcgcaaggtcgaggagaccctgcactacactttcaggcacggtttcgtcaggcaggcctgcaagatctccaacgtggagctgaccaacctggtctcctgcctggggatcctgcacgagaaccgcctggggcagaccgtgctccactctaccctgaagggcgaggcgcgtcgggactatgtccgcgactgcgtctttctctttctctgccacacatggcaagcagccatgggcgtgtggcagcagtgtctcgaggacgagaacctgaaggagctggacaagcttcttgctagaaaccttaaaaagctgtggacgggcttcgacgagcgcaccgttgcctcggacctggccgagatcgtcttccccgagcgcctgaggcagacgctgaaaggcgggctgcccgacttcatgagccagagcatgttgcaaaactaccgcactttcattctcgagcgatctggaatgctgcccgccacctgcaacgccttcccctccgactttgtcccgctgagctaccgcgagtgtcccccgccgctgtggagccactgctacctcttgcagctggccaactacatctcctaccactcggacgtgatcgaggacgtgagcggcgaggggcttctcgagtgccactgccgctgcaacctgtgctccccgcaccgctccctggtctgcaacccccagctccttagcgagacccaggtcattggtaccttcgagctgcaaggtccgcaggagtccaccgctccgctgaaactcacgccggggttgtggacttccgcgtacctgcgcaaatttgtacccgaggactaccacgcccacgagataaagttcttcgaggaccaatcgcgccctcagcacgcggatctcacggcctgcgtcatcacccagggcgcgatcctcgcccaattgcacgccatccaaaaatcccgccaagagtttcttctgaaaaagggtagaggggtctacctggacccccagacgggcgaggtgctcaacccgggtctcccccagcatgccgaggaagaagcaggagccgctagtggaggagatggaagaagaatgggacagccaggcagaggaggacgaatgggaggaggagacagaggaggaagaattggaagaggtggaagaggagcaggcaacagagcagcccgtcgccgcaccatccgcgccggcagcccctccggtcacggatacaacctccgcagctccggccaagcctcctcgtagatgggatcgagtgaagggtgacggtaagcacgagcggcagggctaccgatcatggagggcccacaaagccgcaatcatcgcctgcttgcaagactgcggggggaacatcgctttcgcccgccgctacctgctcttccaccgcggggtgaacatcccccgcaacgtgttgcattactaccgtcaccttcacagctaagaaaaagcaagtaagaggagtcgccggaggaggaggaggcctgaggatcgcggcgaacgagccctcgaccaccagggagctgaggaaccggatcttccccactctttatgccatttttcagcagagtcgaggtcagcagcaagagctcaaagtaaaaaatcggtctctgcgctcgctcacccgcagttgcttgtaccacaaaaacgaagatcagctgcagcgcactctcgaagacgccgaggctctgttccacaagtactgcgcgctcactcttaaagactaaggcgcgcccacccggaaaaaaggcgggaattacctcatcgccaccatgagcaaggagattcccaccccttacatgtggagctatcagccccagatgggcctggccgcgggcgcctcccaggactactccacccgcatgaactggctcagtgccggcccctcgatgatctcacgggtcaacggggtccgcagtcatcgaaaccagatattgttggagcaggcggcggtcacctccacgcccagggcaaagctcaacccgcgtaattggccctccaccctggtgtatcaggaaatccccgggccgactaccgtactacttccgcgtgacgcactggccgaagtccgcatgactaactcaggtgtccagctggccggcggcgcttcccggtgcccgctccgcccacaatcgggtataaaaaccctggtgatccgaggcagaggcacacagctcaacgacgagttggtgagctcttcgatcggtctgcgaccggacggagtgttccaactagccggagccgggagatcctccttcactcccaaccaggcctacctgaccttgcagagcagctcttcggagcctcgctccggaggcatcggaaccctccagttcgtggaggagtttgtgccctcggtctacttcaaccccttctcgggatcgccaggcctctacccggacgagttcataccgaacttcgacgcagtgagagaagcggtggacggctacgactgaatgtcccatggtgactcggctgagctcgctcggttgaggcatctggaccattgccgccgcctgcgctgctttgcccgggagagctgcggactcatctactttgagtttcccgaggagcaccccaacggccctgcacacggagtgcggatcaccgtagagggcaccaccgagtctcacctggtcaggttcttcacccagcaacccttcctggtcgagcgggaccggggcgccaccacctacaccgtctactgcatctgtcctaccccgaagttgcatgagaatttttgctgtactctttgtggtgagtttaataaaagctgaaataagactctactctgggatccagtgtcgtcatcaaatccacaagaccatcaacttcacctttgaggaacaggtgaactttacctgcaagccacacaagaagtacatcacctggttttaccagaacactactctagcagtagccaacacctgctcgaacgacggtgttcttcttccaaacaatctcaccagtggactaactttctcagtgaaaagggcaaagctaattcttcatcgccctattgtagaaggaacttaccagtgtcagagcggaccttgcttccacactttcactttggtgaacgttaccggcagcagcacagccgctccagaaactaaccttccttctgatactaacaaacctcgtttcggaggtgagctttgggttccatctctgacagagggtgggagtcatattgaagtggttgggtatttgattttaggggtggtcctgggtgggtgcatagcagtgctatatcaacttccttgctgggtagaaatcagggtctttatctgctgggtcagacattgtggggaggaaccatgaaggggatcttgctgattatcctttccctggtggggggtgtactggcatgccacgaacagccacgatgtaacatcaccacaggcaatgagaggagtgtaatatgtacagtagtcatcaaatgcgagcataaatgtcctctcaacatcacattcaagaataagaccatgggaaatgcatgggtgggcgattgggaaccaggagatgagcagaactacacggtcactgtccatggtagcgatggaaatcacacttttggtttcaaattcatttttgaagtcatgtgtgatatcacactgcatgtggctagacttcatggcttgtggccccctaccaaggacaacatggttgggttttctttggcttttgtgatcatggcctgcttgatgtcaggtctgctggtaggggctctagtgtggttcctgaagcgaaagcccaggtacggaaatgaggagaaggaaaaattgctataaatctttttctcttcgcagaaccatgaatactttgaccagtgtcgtgctgctctctcttcttgtggtttttagtcagggaaaaatagatagtgaagatgttattggtcattggggtaaaaatataacactagttggaccgacagaaaaacctattgaatggcatggaccaagagttcagctttgcgatggtccaaaaatcttacatccagaatttaatcacacctgtaatgaacagaatcttactctgatattcttgaacaacagttttaatggaaaatactatggtataagaaaggatgggtttggaatgaaacagtacaatcttaaggttattgctccaaaagcttccactcgtaaacctctttccccgcctaagcaaattgatgtcaaaatgggacaaaatgtaactctagttgggccagtagatactccagttaattggcatggaccaaaacatgaactatgcagaggaaatcaaataatacatccagaagttaatcatacatgcaatgaacagaacctcacattgctgtttgttaactacactttctggggagcatatcttggctttgacagatatggtactgacagagtgcattatgaggttacaataatagatggatttgaaaatgcagggcaacaaaaatatgatgagacaagtcagcacaagcctagcaataaagatagaccaagtccaaaagtaaaaaatcctcagaaaacaaaaaacacacacaagacaaacacgcagaacaaaaaggatattgataaagattttccaagaggatctaatcaaactcttgtgggacctcctggttcaaaagttgactggtatgaaggtaaaaatggtgaccttgtaaaactctgtgatggaaagtctggtttaaaggtttcatgcaatgatcaaaacatcactttgattaatgtgaatgaaacctatgctggaacttattatggttctaacaatgacgaccatagacagtatagagtcactgtctatacaataccgcgtaataaaactgttaaaattcaacctcataccacaaaaggaaccacagggggtgccacagttaatgaacagtttgctctgcatcaaggtaatgataaaaccaatcaagatgatgaacaaattccatcaactactgtggcaatcgtggtgggtgtgattgcgggcttcataactataatcattgtcattctgtgctacatctgctgccgcaagcgtcctagggcatacaatcatatggtagacccactactcagcttctcttactgagactcagtcactctcatttcagaaccatgaaggctttcacagcttacgttctgtttagcttagtcacactcattgcagctgcaggatatactcaaattagcatacctagaggtggcagcattacattaaatggtacttttaaaaatactacatggacaagatatcacacaaatggttggaaaaaaatttgtgaatggaatgttacagcttataaatgccacaataatggaagcattactattactgctacaaatattacttctggcagatacaaagctgacagttacaaaaaagaaattagaacttcattttttagaaataataaaactacatttgaagattctggaaattatgaacagcagagattgactttatttaatctaacaataattgagccgccaactactaaggcacccactaccactaaaccaaccacagttaggacaactagagaaacaaccacacagcctactactgtgcccaccacagccagtacaactattgagaccactacccacactacacagttagacactacagtacaaaatagcactgtgttggttaggtatttattgagggaggaaagtactactgaacaaacagaggctacctcaagtgccttcagcagcactgcaaatttaacttcgcttgcttcaataaatgagaccctcgtgccgatgatgcttgaagaacatagaaatccaggtttggatatgcaaattactttcctgattgtctgtggggtctttattcttgtggttcttctttactatgtcttttgcaaggccagacaaaaatctcatagaacaatctacaggccagtgattggggaacctcagccactccaagtggacggaggcttaaggaatcttctcttctcttttacagtatggtgatcagccatgattcctaggttcttcctatttaacatcctcttctgtctcttcaacgtgtgcgctgccttcgcggccgtctcgcacgcctcacccgactgtctcgggcccttccccacctaccttctctttgccctgctcacctgcacctgcgtctgcagcattgtctgcctggtcgtcaccttcctgcagctcatcgactggtgctgcgcgcgctacaattatctccaccacagtcccgaatacagggacgagaacgtagccagaatcttaaggctcatttgaccatgcagactatgctcatactgctatccctcctctcccctgccatcgctgatgatgattactctaaatgcaaattcgcggacatattgaatttcttagaatgctatcaggagaaaatggatatgccatcctattacttggtgattgttggggtagtcatggtctgctcctgcactttctttgccatcatgatctacccctgttttgatctcggctggaactctgttgaggcattcacatacacactagaaaacagttcactagcctccacgccaccacccacaccgcctccccgcagaaatcagttccccctgattcagtacttagaagagccccctccccggcccccttccactgttagctactttcacataaccggcggcgatgactgaccaccacctggacctcgagatggacggccaggcctccgagcagcgcatcctgcaactgcgcgttcgtcagcagcaggagcgggccgccaaggagctcctcgatgccatcaacatccaccagtgcaagaaaggcatcttctgcctggtcaaacaggcaaagatcacctacgagctcgtgtccggcggcaagcagcatcgcctcgcctatgagctgccccagcagaagcagaaattcacctgcatggtgggcgtcaaccccatagtcatcacccagcagtcgggcgagaccagcggctgcatccactgctcctgcgaaagccccgagtgcatctactccctcctcaagaccctttgcggactccgcgacctcctccccatgaactgatgttgattaaaacccagaaaccaatcagcccattcccccatttcccattcccaattactcataagaataaatcattggaattaatcattcaataaagatcacttacttgaaatctgaaagtatgtctctggtgtagttgtttagcagcacctcggtcccctcctcccagctctggtactccagtccccggcgggcggcgaacttcctccacaccttgaaagggatgtcaaattcctggtccacaattttcattgtcttccctctcagatgtcaaagaggctccgggtggaagatgacttcaaccccgtctacccctatggctacgcgcggaatcagaatatccccttcctcactcccccctttgtctcctccgatggattccaaaacttcccccctggggtgctatcactcaaactggctgacccaatcaccatcaccaatgggaatgtctcactcaaggtgggaggggggctcaccttgcaagaaggaactggggacctaaaggtgaacgctaagtcccccttgcaagttgcaactaataaacagttggagattgcacttgctaagccatttgaggaaaaggatggcaaacttgctttaaaaattggccatggattagccgttgtggatgaaaatcatactcacttacaatcactaataggtacacttgttattttaactggcaagggaattggtacaggtagagctgaaagtggaggaactatagatgtaagacttggaagtggaggaggtttgtcatttgataaagacggaaacctagtagcctggaacaaagatgatgataggagaactctttggacaacgccagacccatctccaaattgcaaaattgatcaggacaaagactccaaacttacttttgtattaactaaatgtgggagtcagattttggctaatatgtctttgctggtagttaaaggaaaattttccatgataaataacaaagtaaatggaactgatgattataaaaaatttacaataaagttattatttgatgaaaagggtgttttattaaaagactctagtcttgataaagaatattggaactatagaagcaataataacaatgttggaagtgcatatgaagaagctgttggttttatgccaagtacaacagcttatcctaaaccacctactcctcctactaatcctacaactcctttagaaaagagtcaagcaaaaaataaatacgtaagtaacgtctaccttggcggacaagcaggcaacccagttgccacaactgttagttttaataaggaaacggggtgtacttattcgattacatttgactttgcttggaacaaaacttatgaaaatgtgcagtttgattcctcctttttaactttctcctatattgcccaagaatgaaagaccaataaacgtgtttttcatttgaaaatttcatgtatctttattgatttttacaccagcacgggtagtcagtctcccaccaccagcccatttcacagtgtacacggttctctcagcacgggtggccttaaataggggaatgttctgattagtgcgggaactggatttagtgtctataagccacacagtttcctggcgagccaaacggggatcggtgattgagatgaatccgtcctctgaaaagtcttccaagcgggcctcgcagtccaaagtcacagtctggtggaatgagaagaacgcacagattcatactcggaaaacaggatgggtctgtgcctctccatcagcgccctcaacagtctttgccgccggggctcggtgcggctgctgcagatgggatcgggatcgcaagtctctctgactatgatccccacagccttcagcatcagtctcctggtgcgtcgggcacagcaccgcatcctgatctcgctcatgttctcacagtaagtgcagcacattatcaccatgttattcagcagcccataattcagggtgctccagccaaagctcatgttggggatgatggaacccacgtgaccatcgtaccagatgcggcagtatatcaggtgcctgcccctcatgaacacactgcccatatacatgatctctttgggcatgtttctgttcacaatctgccggtaccaggggaagcgctggttgaacatgcacccgtaaatgactctcctgaaccacacggccagcagggtgcctcccgcccgacactgcaaggagcccggggatgaacagtggcaatgcaggatccagcgctcgtacccgctcaccatctgagctctcaccaagtccagggtagcggggcacaggcacactgacatacatctttttaaaatttttatttcctctggggtcaagatcatatcccaggggactggaaactcttggagcagggtaaagccagcagcacatggtaatccgcggacagaacttacattatgataatctgcatgatcacaatcgggcaacagagggtgttgttcagtcagagaggccctggtctcctcatcagatcgtggtaaacgggccctgcgatatggatgatggcggagcaagctcgactgatcctcggtttgcattgtagtggattctcttgcgtaccttgtcgtacttctgccagcagaaatgagcccttgaacagcagatacctctccttctcctgtctttccgctgctgacgctcagtcatccaactgaagtacagccattcccgcaggttctcgagcagctcctcagcatctgatgaaacaaaagttctgtccatgcggattccccttaacacatcagccaggacattgtaggccatcccaatccagttaatgcagcctggtctatcattcagaggaggtgggggaagaactggaagaaccatttttattccaagcggtctcgaaggacgataaagtgcaagtcacgcaggtgacagcgttccccgccgctgtgctggtggaaacagacagccaggtcaaaacctactctattttcaaggtgctcgactgtggcttcgagcagtggctctacgcgtacatccagcataagaatcacattaaaggctggccctccatcgatttcatcaatcatcaggttacactcattcaccattcccaggtaattctcatttttccagccttgaattatttctacaaattgttggtgtaagtccactccgcacatgtggaaaagttcccacagcgccccctccactttcataatcaggcagaccttcataatagcaacagatctggctgctccaccacctgcagcgtgttcaaaacaacaagattcaatgagtttctgccctctgccctgagctcgcgtctcagcgtcagctgtaaaaagtcactcaagtcctcggccactacagatgccaattcagagccagggctaagcgtgggactggcaagcgtgatggagtactttaatgctccaaagctagcacccaaaaactgcacgctggaataagctctctttgtgtcaccggtgattccttccaagaggtgagtgataaagcgaggtaggtgctctctaatcatagcagtaatggaaaagtcctctaaataagtcactagggccccagggaccacaatgtggtagctgacagcgcgtcgctcaagcatggttagtagagatgagagtctgaaaaacagaaagcatgcactaaaccagagtggcaagtcttactgaaggaaaaatcactctctccagcagcaaagtgcccactgggtggccctctcggacatacaaaaatcgatccgtgtggttaaagagcagcacagttagctcctgtcttctcccagcaaagatcacatcggactgggttagtatgcccctggaatggtagtcattcaaggccataaatctgccttggtagccattaggaatcagcacgctcactctcaagtgaaccaaaaccaccccatgcggaggaatgtggaaagattcggggcaaaagaaattatatctattgctagtcccttcctggacgggagcgatccctccagggctatctatgaaagcatacagagattcagccatagctcagcccgcttaccagtagacagagagcacagcagtacaagcgccaacagcagcgactgactacccactaacccagctccctatttaaaggcaccttacactgacgtaatgaccaaaggtctaaaaaccccgccaaaaaaaacacacacgccctgggtgtttttcgcgaaaacacttccgcgttctcacttcctcgtatcgatttcgtgactcaacttccgggttcccacgttacgtcacttctgcccttacatgtaactaagccgtagggcgccatcttgcccacgtccaaaatggcttccatgtccggccacgcctccgcggcgaccgttgacccgtgcgtcgtgacgtcatttgcgtcatcttctcttgtccaatcagcgtcggccacgccctaaattcaaaagctcatttgcatattaacttttgtttactttgtggggtatattattgatgatg

SEQ ID NO: 3 - DNA, 40 bp, Artificial
  Synthetic oligonucleotide Ad48 (1-462) forw
cagaatttaattaatcgacatcatcaataatataccccac

SEQ ID NO: 4 - DNA, 36 bp, Artificial
  Synthetic oligonucleotide Ad48 (1-462) rev
cagaatcgcctaggtcagctgatctgtgacataaac

SEQ ID NO: 5 - DNA, 35 bp, Artificial
  Synthetic oligonucleotide Ad48 (3362-5909) forw
cagaatcgggatccaggtaggttttgagtagtggg

SEQ ID NO: 6 - DNA, 44 bp, Artificial
  Synthetic oligonucleotide Ad48 (3362-5909) rev
cagaatacgcgtcgacttaattaatctcgagagggaatacctac

SEQ ID NO: 7 - DNA, 33 bp, Artificial
  Synthetic oligonucleotide SfiI-forw
cagaatttaattaacatgacagcgacgagactg

SEQ ID NO: 8 - DNA, 38 bp, Artificial
  Synthetic oligonucleotide Ad48 (3362-5909) rev
acgcgtcgacttaattaatctcgagagggaatacctac

SEQ ID NO: 9 - DNA, 21 bp, Artificial
  Synthetic oligonucleotide SbfI-forw
tggagatggaagatgcaactc

SEQ ID NO: 10 - DNA, 32 bp, Artificial
  Synthetic oligonucleotide FseI-Rpac
cagaatttaattaacagccgaaggcgagccag

SEQ ID NO: 11 - DNA, 31 bp, Artificial
  Synthetic oligonucleotide MluI-FseI
cagaatggccggcctctacgcgtacatccag

SEQ ID NO: 12 - DNA, 36 bp, Artificial
  Synthetic oligonucleotide rITR-R
cagaatttaattaacatcatcaataatataccccac

SEQ ID NO: 13 - DNA, 35 bp, Artificial
  Synthetic oligonucleotide SbfI-Fpac
cagaatttaattaatggagatggaagatgcaactc

SEQ ID NO: 14 - DNA, 18 bp, Artificial
  Synthetic oligonucleotide FseI-R
cagccgaaggcgagccag

SEQ ID NO: 15 - DNA, 20 bp, Artificial
  Synthetic oligonucleotide dE3AscI-F1
aaagactaaggcgcgcccac

SEQ ID NO: 16 - DNA, 33 bp, Artificial
  Synthetic oligonucleotide Ad48dE3SpeIR1
cagaatactagtgcaggtgttggctactgctag

SEQ ID NO: 17 - DNA, 36 bp, Artificial
  Synthetic oligonucleotide Ad48dE3SpeIF2
cagaatactagtccatgaactgatgttgattaaaac

SEQ ID NO: 18 - DNA, 20 bp, Artificial
  Synthetic oligonucleotide Ad48dE3SnaBI-R
tccgccaaggtagacgttac

SEQ ID NO: 19 - DNA, 21 bp, Artificial
  Synthetic oligonucleotide Ad48.SnaBI-F
tcctactaatcctacaactcc

SEQ ID NO: 20 - DNA, 31 bp, Artificial
  Synthetic oligonucleotide Ad48E4orf7-R
gggagaaaggactgtgtacactgtgaaatgg

SEQ ID NO: 21 - DNA, 30 bp, Artificial
  Synthetic oligonucleotide Ad48/Ad5E4orf6-F
cacagtgtacacagtcctttctccccggct

SEQ ID NO: 22 - DNA, 29 bp, Artificial
  Synthetic oligonucleotide Ad48/Ad5E4orf6-R
agaatccactacaatgactacgtccggcg

SEQ ID NO: 23 - DNA, 30 bp, Artificial
  Synthetic oligonucleotide Ad48E4orf4-F
ggacgtagtcattgtagtggattctcttgc

SEQ ID NO: 24 - DNA, 20 bp, Artificial
  Synthetic oligonucleotide Ad26MluI-R
gatgtacgcgtagagccact

SEQ ID NO: 25 - DNA, 36 bp, Artificial
  Synthetic oligonucleotide Ad49 (1-462) forw
ccttaattaatcgacatcatcaataatataccccac

SEQ ID NO: 26 - DNA, 30 bp, Artificial
  Synthetic oligonucleotide Ad49 (1-462) rev
cgcctaggtcagctgatctgtgacataaac

SEQ ID NO: 27 - DNA, 32 bp, Artificial
  Synthetic oligonucleotide Ad26 (3365-5913)-F
cagaagggatccaggtaggtttgagtagtggg

SEQ ID NO: 28 - DNA, 40 bp, Artificial
  Synthetic oligonucleotide Ad26 (3365-5913)-R
caacgcgtcgacttaattaatcttgagagggaatacctac

SEQ ID NO: 29 - DNA, 32 bp, Artificial
  Synthetic oligonucleotide MluI-F
cagaatcctgcaggctctacgcgtacatccag

SEQ ID NO: 30 - DNA, 36 bp, Artificial; identical to SEQ ID NO: 12
  Synthetic oligonucleotide rITR-R

SEQ ID NO: 31 - DNA, 34 bp, Artificial
  Synthetic oligonucleotide SrfI-F
cagaatttaattaaactatgccagacgcaagagc

SEQ ID NO: 32 - DNA, 18 bp, Artificial
  Synthetic oligonucleotide SbfI-R
ctcgtacgagggcggctc

SEQ ID NO: 33 - DNA, 20 bp, Artificial
  Synthetic oligonucleotide dE3AscI-F1
aaagactaaggcgcgcccac

SEQ ID NO: 34 - DNA, 33 bp, Artificial
  Synthetic oligonucleotide dE3SpeI-R1
cagaatactagtgcagtgagtgttggagactgc

SEQ ID NO: 35 - DNA, 36 bp, Artificial
  Synthetic oligonucleotide dE3SpeI-F2
cagaatactagtccatgaactgatgttgattaaaag

SEQ ID NO: 36 - DNA, 23 bp, Artificial
  Synthetic oligonucleotide Ad26dE3EcoRI-R2
gatggtaatagaattccattctc

SEQ ID NO: 37 - DNA, 23 bp, Artificial
  Synthetic oligonucleotide Ad26.EcoRI-F
gagaatggaattctattaccatc

SEQ ID NO: 38 - DNA, 31 bp, Artificial
  Synthetic oligonucleotide Ad49E4orf7-R
gggagaaaggactgtttacactgtgaaatgg

SEQ ID NO: 39 - DNA, 30 bp, Artificial
  Synthetic oligonucleotide Ad5E4orf6-F
cacagtgtaaacagtcctttctccccggct

SEQ ID NO: 40 - DNA, 29 bp, Artificial
  Synthetic oligonucleotide Ad26/Ad5E4orf6-R
agaatccatttcaatgactacgtccggcg

SEQ ID NO: 41 - DNA, 30 bp, Artificial
  Synthetic oligonucleotide Ad26E4orf4-F
ggacgtagtcattgaaatggattctcttgc

SEQ ID NO: 42 - DNA, 20 bp, Artificial
  Synthetic oligonucleotide Ad26MluI-R
gatgtacgcgtagagccact

SEQ ID NO: 43 - protein, 11 aa, Artificial
  AL11 HIV epitope
AAVKNWMTQTL

SEQ ID NO: 44 - protein, 9 aa, Artificial
  KV9 HIV epitope
KSLYNTVCV

SEQ ID NO: 45 - protein, 13 aa, Artificial
  DD13 HIV epitope
DRFYKSLRAEQTD